(19) United States
(12) Patent Application Publication
(10) Pub. No.: US 2002/0052028 A1
(43) Pub. Date: May 2, 2002

(54) Bio-intermediates for use in the chemical synthesis of polyketides

(76) Inventors: Daniel Santi, San Francisco, CA (US); Gary Ashley, Alameda, CA (US); David C. Myles, Kensington, CA (US)

Correspondence Address: Carolyn A. Favorito, Morrison & Foerster LLP, Suite 500, 3811 Valley Centre Drive, San Diego, CA, 92130-2332, US

(21) Appl. No.: 09/927,559

(22) Filed: Aug. 9, 2001

Related U.S. Application Data
(63) Continuation-in-part of application No. 09/867,845, filed on May 29, 2001.
(60) Provisional application No. 60/248,387, filed on Nov. 13, 2000.
(60) Provisional application No. 60/224,038, filed on Aug. 9, 2000.
(60) Provisional application No. 60/237,382, filed on Oct. 4, 2000.

(51) Int. Cl. C12P 17/16; C12P 19/62

(52) U.S. Cl. 435/76; 435/118

(57) ABSTRACT

The present invention relates to compounds made by a subset of modules from one or more polyketide synthase (“PKS”) genes that are used as starting material in the chemical synthesis of novel molecules, particularly naturally occurring polyketides or derivatives thereof. The biologically derived intermediates (“bio-intermediates”) generally represent particularly difficult compounds to synthesize using traditional chemical approaches due to one or more stereocenters. In one aspect of the invention, an intermediate in the synthesis of epothilone is provided that feeds into the synthetic protocol of Danishefsky and co-workers. In another aspect of the invention, intermediates in the synthesis of discodermolide are provided that feed into the synthetic protocol of Smith and co-workers. By taking advantage of the inherent stereochemical specificity of biological processes, the syntheses of key intermediates and thus the overall syntheses of compounds like epothilone and discodermolide are greatly simplified.

This application claims priority to U.S. provisional applications No. 60/248,387, filed Nov. 13, 2000; 60/224,038, filed Aug. 9, 2000; and 60/237,382, filed Oct. 4, 2000. This application is a continuation-in-part of U.S. patent application Ser. No. 09/867,845, filed May 29, 2001, by inventors Santi, Siani, Khosla, and Reid (attorney docket no. 30062-20055.00) and PCT patent application No. US01/17352, which claim priority to U.S. provisional application 60/207,331, now lapsed. Each of the foregoing patent applications is incorporated herein by reference.

### BACKGROUND

Produced naturally in many type of organisms including fungi and mycelial bacteria (particularly actinomycetes), polyketides are a structurally diverse class of compounds that are the source of many biologically active molecules. Two examples of polyketides that are of particular recent interest include epothilone (particularly epothilone D) and discodermolide.

[chemical structure]

Initial studies of these compounds in tubulin polymerization assays suggest they may act as potent anti-cancer agents. However, more extensive on going clinical investigations are hampered by the small quantities of epothilone and discodermolide that can be obtained from naturally occurring sources.

Several research groups have succeeded in the de novo chemical syntheses of epothilone and discodermolide. Syntheses of epothilones are described in, for example, Danishefsky et al., “Synthesis of epothilones, intermediates thereto and analogues thereof,” U.S. Pat. Nos. 6,204,388 and 6,242,469 (both of which are incorporated herein by reference). Syntheses of discodermolide are described in, for example, Smith et al., “Synthetic techniques and intermediates for polyhydroxy, dienyl lactones and mimics thereof,” U.S. Pat. Nos. 5,789,605 and 6,031,133; and Smith et al., “Synthetic techniques and intermediates for polyhydroxy, dienyl lactone derivatives,” U.S. Pat. Nos. 6,096,904 and 6,242,616 (each of which is incorporated herein by reference). The reported syntheses are long, complex, and generally not amenable for making commercial quantities of these compounds. Because there are many other polyketides of interest that are hampered by inadequate supply, and because epothilone and discodermolide could be more readily developed for therapeutic and other uses if a more adequate supply were available, a need exists for novel approaches for obtaining these important compounds.

### SUMMARY

The present invention provides methods for making modular polyketide synthases and genes that encode them for the production of polyketides of defined structure. Such polyketides are useful as intermediates in the chemical synthesis of more complex polyketides, or they may be useful in their own right. The inherent stereochemical specificities of biological processes result in highly efficient production of optically-active intermediates for use in the chemical synthesis of complex polyketides. Because intermediates with complex stereochemical centers are more readily synthesized in optically-pure form using these biological strategies, polyketides may be chemically synthesized more simply and economically by these methods.

In one aspect of the invention, a method for designing a gene for making a particular polyketide compound is provided. The method comprises:

defining the compound as a sequence of two-carbon units;

comparing the two-carbon unit sequence of the compound with a database of naturally occurring PKS structures wherein each database PKS structure is also described as a sequence of two-carbon units;

for each two-carbon unit of the compound, searching the database for a matching two-carbon unit;

for each two-carbon unit of the compound for which a match was found in the database, associating a PKS gene fragment corresponding to the matched database two-carbon unit; and,

designing a new gene capable of producing said compound wherein the gene includes the PKS gene fragments associated with the matched database two-carbon units.

In a second aspect of the invention, genes encoding novel polyketide synthases (PKSs) which catalyze the formation of desired polyketides are provided. These genes comprise a collection of fragments of natural PKS genes, each fragment encoding at least a module of a PKS, or the ketosynthase, acyltransferase, and acyl-carrier protein domains of a module, capable of catalyzing the formation of a designated 2-carbon unit in the desired polyketide. Said gene fragments may be genetically engineered so as to alter the domain content of the resulting PKS module, so as to provide the desired polyketide. In preferred embodiments, the PKS gene fragments are associated with a coding sequence for a terminal thioesterase domain, and are placed in expression vectors.

In another aspect of the invention, the genes encoding novel PKSs are introduced into host cells which support the production of the desired polyketides during fermentation. In preferred embodiments, the host cells either do not their native PKS genes deleted. In particularly preferred embodiments, the host cells are Streptomyces coelicolor, Streptomyces lividans, Streptomyces fradiae, Saccharopolyspora erythraea, Escherichia coli, Myxococcus xanthus, or Saccharomyces cerevesiae.

In another aspect of the invention, the novel polyketides produced from the above host cells are provided.

In another aspect, the present invention provides a method for making a first compound useful in synthesizing a second compound, wherein said second compound contains four or more chiral centers, and said first compound contains two or more chiral centers, said method comprising expressing in a recombinant host cell a recombinant, non-naturally occurring polyketide synthase that produces said first compound. In preferred embodiments, the first compound contains at least 3 chiral centers, and the second compound contains at least 5 chiral centers. In a particularly preferred embodiment, the second compound contains at least 10 chiral centers. In a preferred embodiment, said first and second compounds are polyketides. The recombinant, non-naturally occurring PKS can be either a portion of a naturally occurring PKS gene or can be composed of portions of two or more naturally occurring PKS genes. The portions of the two PKS genes can each comprise two or more extender modules. In a preferred embodiment, the second compound is a naturally occurring polyketide, and the non-naturally occurring recombinant PKS is derived from one or more PKS that does or do not produce the second compound.

In another aspect of the invention, a combination of biological and chemical methods for the synthesis of epothilone and epothilone analogs is provided. Intermediate compounds and methods for making the same are provided that are used as starting materials in the chemical synthesis of epothilones.

In another aspect of the invention, a combination of biological and chemical methods for the synthesis of discodermolide and discodermolide analogs is provided. Intermediate compounds and methods for making the same are provided that are used as starting materials for use in the chemical synthesis of discodermolide.

Definitions

Listed below are definitions of various terms used to describe this invention. These definitions apply to the terms as they are used throughout this specification, unless otherwise stated in specific instances, either individually or as part of a larger group.

The term “polyketide” refers to a compound that can be derived by the decarboxylative condensation of a succession of malonyl thioester extender units onto a starting acyl thioester. The malonyl thioesters may be optionally substituted, for example, methylmalonyl, ethylmalonyl, methoxymalonyl, hydroxymalonyl, and the like. Examples of starting acyl thioesters includes but is not limited to alkanoates such as acetyl, propionyl, butyryl, isobutyryl, sec-valeryl, and the like; cycloalkanoates such as cyclohexanoyl; alkenoates such as acryloyl and crotonoyl; cycloalkenoates, such as cyclohexenoyl; and aryl, such as benzoyl, thiazolyl, and the like. After condensation, the extender units may be further modified by redox chemistry, methylation, and other transformations. Polyketides may be either of natural origin and produced by naturally-occurring polyketide synthases, may be the products of genetically-engineered polyketide synthases either in vivo or in vitro, or may be produced by chemical synthesis. When produced by chemical synthesis, methods other than the decarboxylative condensation of a succession of malonyl thioester extender units onto a starting acyl thioester may be employed for production of polyketides.

The term “polyketide synthase” (“PKS”) refers to an enzyme catalyzing the biosynthesis of a polyketide. The term “modular PKS” refers to a class of PKS wherein each step in the biosynthesis of a complex polyketide is catalyzed by a separate domain of the enzyme, and said domains are arranged in a predictable order along the polypeptide chain. Examples of naturally-occurring polyketide synthases include but are not limited to those involved in the biosynthesis of erythromycin (ery), megalomicin (meg), pikromycin (pik), narbomycin (nar), oleandomycin (ole), lankamycin (1 km), FK506 (506), FK520 (asc), rapamycin (rap), epothilone (epo), tylosin (tyl), spiramycin (spm), rosamicin (rsm), geldanamycin (gdm), pimaricin (pim), FR008 (fr8), candicidin (can), avermectin (avr), tartralone (tar), borophycin (bor), aplasmomycin (apl), boromycin (brm), discodermolide (dsc), and the like.

The term “recombinant” refers to genes, proteins, or organisms which have been genetically engineered. An example of a recombinant gene is a DNA sequence which has been cloned from its original source and optionally modified so as to alter the coding sequence. An example of a recombinant protein is a protein which is expressed from a recombinant gene. An example of a recombinant organism is an organism which contains recombinant genes.

The term “module” refers to a contiguous segment of a PKS polypeptide containing the domains necessary for the addition and processing of a single extender unit onto the polyketide. A PKS module contains a core of three domains, including a ketosynthase, an acyltransferase, and an acyl-carrier protein domain. A module may also contain further domains involved in processing the added extender unit. A listing of the most common module types and their polyketide structural outcomes is given in FIG. 8.

The term “domain” refers to a portion of a PKS catalyzing a single step in the biosynthesis of a polyketide. Examples of domains include but are not limited to ketosynthases (KS), acyltransferases (AT), acyl-carrier protein (ACP), ketoreductase (KR), dehydratase (DH), enoylreductase (ER), C-methyltransferase (MT), 0-methyltransferase (OMT), and thioesterase (TE). The common order of domains within a module is KS-AT-[modification domains]-ACP. Modification domains occur either singly, for example as a KR or a MT, in pairs, as in DH-KR, or in triplets as in DH-ER-KR.

As used herein, the terms “discodermolides,” “discodermolide compounds,” and “discodermolide analogs” refer to compounds of the formula:

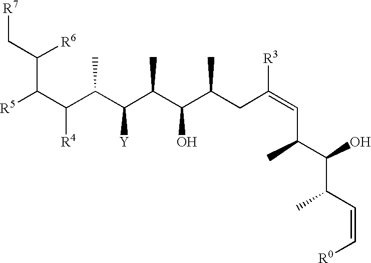

wherein R⁰, R¹, R², R³, R⁴, R⁵, R⁶, R⁷, X, and Y are described herein, and includes analogs derived therefrom that possess microtubule-stabilizing activity in one of the assays described by Bollag et al., Cancer Research 55:2325-2333 (1995) (incorporated herein by reference) or in a comparable assay.

As used herein, the terms “epothilones, ” “epothilone compounds, ” and “epothilone analogs ” refer to compounds of the formula:

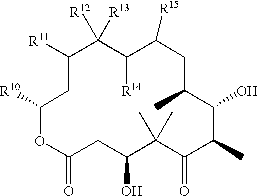

wherein R¹⁰ is alkenyl or aryl, optionally substituted with one or more groups as defined below;

R¹¹ is H;

R¹² is H;

or R¹¹ and R¹² taken together form a bond;

or R¹¹ and R¹² taken together form —O—;

R¹³ is H, alkyl, hydroxyalkyl, or fluoroalkyl;

R¹⁴ is H;

R¹⁵ is H;

or R¹⁴ and R¹⁵ taken together form a bond;

or R¹⁵ and R¹⁵ taken together form —O—;

and includes analogs derived therefrom that possess microtubule-stabilizing activity in one of the assays described by Bollag et al., Cancer Research 55:2325-2333 (1995) (incorporated herein by reference) or in a comparable assay.

In preferred embodiments, R¹⁰ is taken from the set consisting of 1-(2-methylthiazol-4yl)-propen-2-yl, 1-(2-hydroxymethylthiazol-4yl)-propen-2-yl, 1-(2-fluoromethylthiazol-4yl)-propen-2-yl, 1-(2-aminomethylthiazol-4yl)-propen-2-yl, 6-quinolyl, and 2-methylbenzothiazol-5-yl; R¹¹ and R¹² taken together form a bond; R¹³ is methyl, hydroxymethyl, dioxolan-2-ylmethyl, and fluoromethyl; R¹⁴ is H; R¹⁵ is H; or R¹⁴ and R¹⁵ taken together form a bond.

The term “alkyl” refers to straight, branched, or cyclic hydrocarbons, optionally substituted as defined below. Examples of alkyl groups include but are not limited to methyl, ethyl, propyl, isopropyl, isobutyl, cyclopropyl, cyclobutyl, cyclopenty, cyclohexyl, and the like, including substituted forms thereof.

The term “alkenyl” refers to an straight, branched, or cyclic hydrocarbon group containing at least one carbon-carbon double bond, optionally substituted as defined below. Examples of alkenyl groups include but are not limited to vinyl, allyl, cyclohexenyl, and the like, including substituted forms thereof.

The term “alkynyl” refers to an straight, branched, or cyclic hydrocarbon group containing at least one carbon-carbon triple bond, optionally substituted as defined below. Examples of alkynyl groups include but are not limited to ethynyl, propargyl, and the like, including substituted forms thereof.

The term “aryl” refers to an aromatic moiety including heteroaryls having one or more heteroatoms such as N, O, and S, optionally substituted as defined below. Examples of aryl groups include but are not limited to phenyl, pyridyl, pyrimidinyl, pyrrolyl, pyrrazolyl, triazolyl, tetrazolyl, furyl, isoxazolyl, oxazolyl, imidazolyl, thiazolyl, thienyl, indolyl, indazolyl, quinolyl, isoquinolyl, quinoxalyl, phthaloyl, phthalimidoyl, benzimidazolyl, benothiazolyl, benzofuryl, and the like, including substituted forms thereof.

The “alkyl,” alkenyl,” “aryl,” and other moieties may optionally be substituted with one or more substituents. Illustrative examples of substituents include but are not limited to alkyl, alkenyl, alkynyl, aryl, halogen (F, Cl, Br, I); trifluoromethyl; trifluoromethoxy; hydroxy; alkoxy; cycloalkoxy; hetoercyclooxy; oxo; alkanoyl (—C(═O)-alkyl); aryloxy; alkanoyloxy; amino; alkylamino; arylamino; aralkylamino; cycloalkylamino; heterocycloamino; disubstituted amines in which the two amino substituents are selected from alkyl, aryl, or aralkyl; alkanoylamine; aroylamino; aralkanoylamino; substituted alkanoylamino; substituted arylamino; substituted aralkanoylamino; thiol; alkylthio; arylthio; aralkylthio; cycloalkylthio; heterocyclothio; alkylthiono; arylthiono; aralkylthiono; alkylsulfonyl; arylsulfonyl; aralkylsulfonyl; sulfonamido (e.g., SO²NH²); substituted sulfonamido; nitro; cyano; carboxy; carbamyl (e.g., CONH₂); substituted carbamyl (e.g., —C(═O)NRR′ where R and R′ are each independently hydrogen, alkyl, aryl, aralkyl and the like); alkoxycarbonyl, aryl, substituted aryl, guanidino, and heterocyclo such as indoyl, imidazoly, furyl, thienyl, thiazolyl, pyrrolidyl, pyridyl, pyrimidyl and the like. Where applicable, the substituent may be further substituted such as with halogen, alkyl, alkoxy, aryl, or aralkyl and the like. Particularly preferred examples of substituted alkyls include fluoromethyl and fluoroethyl. Particularly preferred examples of substituted aryls include 2-methyl-4thiazolyl, 2-(hydroxymethyl)-4-thiazolyl, 2-(fluoromethyl)-4-thiazolyl, and 2-(aminomethyl)-4-thiazolyl.

The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2nd edition, John Wiley & Sons, New York (1991), which is incorporated herein by reference. Illustrative hydroxyl protecting groups include but not limited to tetrahydropyranyl (THP); benzyl; 4-methoxybenzyl (PMB); methylthiomethyl; ethythiomethyl; pivaloyl; phenylsulfonyl; triphenylmethyl; trisubstituted silyl such as trimethyl silyl (TMS), triethylsilyl (TES), tributylsilyl, tri-isoprylsilyl (TIPS), t-butyldimethylsilyl (TBS), tri-t-butylsilyl, methyldiphenylsilyl, ethyldiphenylsily, t-butyldiphenylsilyl and the like; acyl and aroyl such as acetyl (Ac), pivaloylbenzoyl (Piv), 4-methoxybenzoyl, 4-nitrobenzoyl and aliphatic acylaryl and the like. All hydroxyl groups of compounds described herein may optionally be protected with a hydroxy protecting group.

In addition to the explicit substitutions at the above-described groups, the inventive compounds may include other substitutions where applicable. For example, the discodermolide backbone (e.g., C-1 through C-24) or backbone substituents may be additionally substituted (e.g., by replacing one of the hydrogens or by derivatizing a non-hydrogen group) with one or more substituents such as C₁-C₅ alkyl, C₁-C₅ alkoxy, phenyl, or a functional group. Illustrative examples of suitable functional groups include but are not limited to alcohol, sulfonic acid, phosphine, phosphonate, phosphonic acid, thiol, ketone, aldehyde, ester, ether, amine, quaternary ammonium, imine, amide, imide, imido, nitro, carboxylic acid, disulfide, carbonate, isocyanate, carbodiimide, carboalkoxy, carbamate, acetal, ketal, boronate, cyanohydrin, hydrozone, oxime, hydrazide, enamine, sulfone, sulfide, sulfenyl, and halogen.

Epothilone Compounds of the Invention

In one aspect of the invention, epothilone analogs of the following formula are provided:

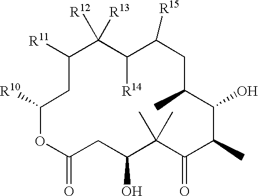

wherein

R¹⁰ is alkenyl or aryl;

R¹¹ is H;

R¹² is H;

or R¹¹ and R¹² taken together form a bond;

or R¹¹ and R¹² taken together form —O—;

R¹³ is H, alkyl, hydroxyalkyl, or fluoroalkyl;

R¹⁴ is H;

R¹⁵ is H;

or R¹⁴ and R¹⁵ taken together form a bond;

or R¹⁴ and R¹⁵ taken together form —O—.

In preferred embodiments, R¹⁰ is taken from the group consisting of 1-(2-methylthiazol-4yl)-propen-2-yl, 1-(2-hydroxymethylthiazol-4yl)-propen-2-yl, 1-(2-fluoromethylthiazol-4yl)-propen-2-yl, 1-(2-aminomethylthiazol-4yl)-propen-2-yl, 6-quinolyl, and 2-methylbenzothiazol-5-yl; R¹¹ and R¹² taken together form a bond; R¹³ is methyl, hydroxymethyl, dioxolan-2-ylmethyl, and fluoromethyl; R¹⁴ is H; R¹⁵ is H; or R¹⁴ and R¹⁵ taken together form a bond.

In a particularly preferred embodiment, the epothilone analog is selected from the group consisting of:

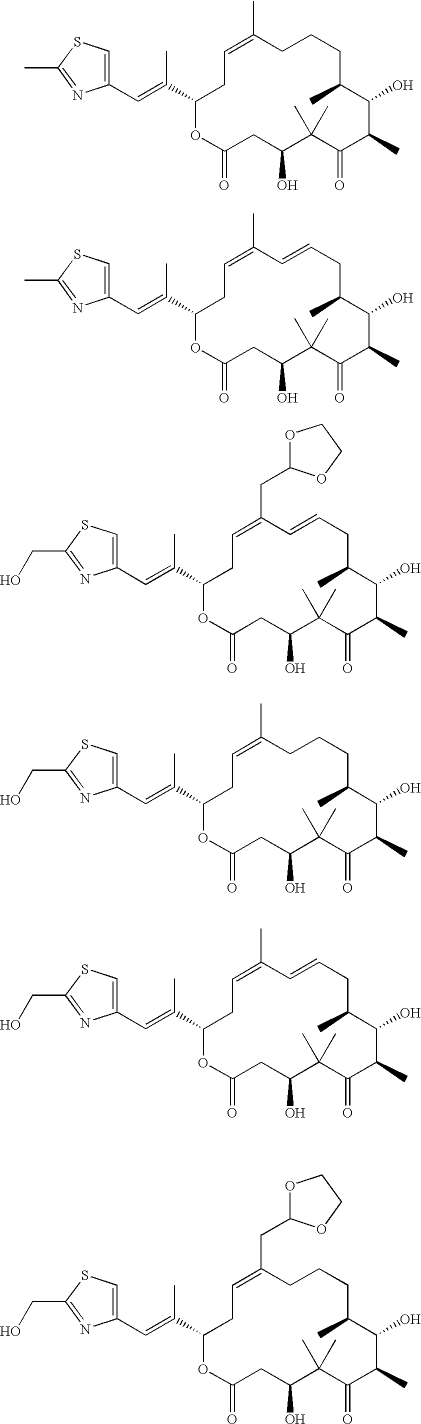

In another aspect of the invention, intermediates leading to the synthesis of the above epothilone analogs are provided. In preferred embodiments, these intermediates are taken from the group consisting of:

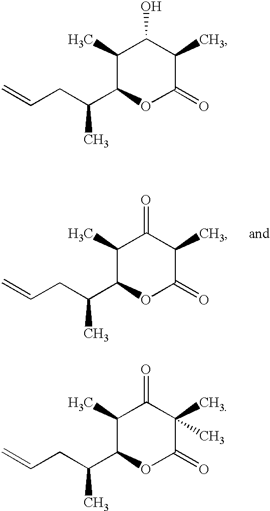

Discodermolide Compounds of the Invention

In one aspect of the present invention, novel discodermolide compounds are provided of the formula:

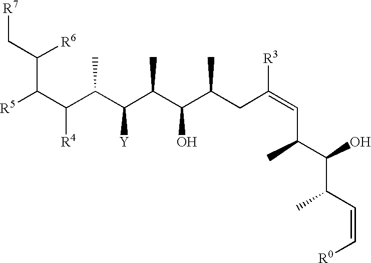

where R⁰ is C1-C8 alkyl, C1-C8 alkenyl, C1-C8 alkynyl, aryl, 2-phenylethyl, 2-(3-hydroxyphenyl)ethyl, or a group of the formula

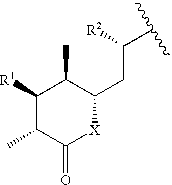

wherein R¹ and R² are each independently hydrogen, hydroxyl, or a hydroxyl protecting group; and X is O, NH, or N-alkyl;

R³ is hydrogen, C₁-C₁₀ alkyl or aryl;

R⁴, R⁵, R⁶, and R⁷ are each hydrogen, or R⁴ and R⁵ together form a double bond and R⁶ and R⁷ together form a double bond; and

Y is hydroxyl, amino, —OC(═O)NH₂ or —NHC(═O)NH₂, with the proviso that when R³ is hydrogen or C₁-C₆ alkyl that: (i) at least one of R¹ and R² is not hydroxyl, or (ii) R⁴, R⁵, R⁶, and R⁷ are each hydrogen, or (iii) X is nitrogen, or (iv) Y is hydroxyl, amino, or —NHC(═O)NH₂, or (v) any combination of (i) through (iv).

In another embodiment of the present invention, compounds are provided of the formula

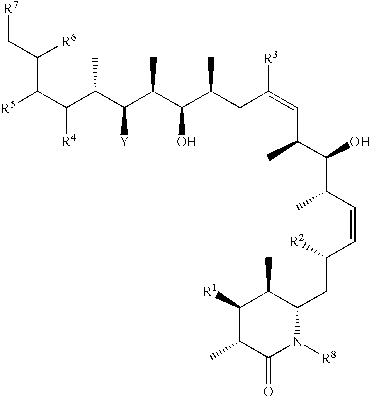

wherein

R¹ and R² are each independently hydrogen, hydroxyl, or a hydroxyl protecting group;

R³ is hydrogen, C₁-C₁₀ alkyl or aryl;

R⁴, R⁵, R⁶, and R⁷ are each hydrogen, or R⁴ and R⁵ together form a double bond and R⁶ and R⁷ together form a double bond;

R⁸ is H or C₁-C₈ alkyl; and,

Y is hydroxyl, amino, —OC(═O)NH₂ or —NHC(═O)NH₂.

In another embodiment of the present invention, compounds are provided of the formula

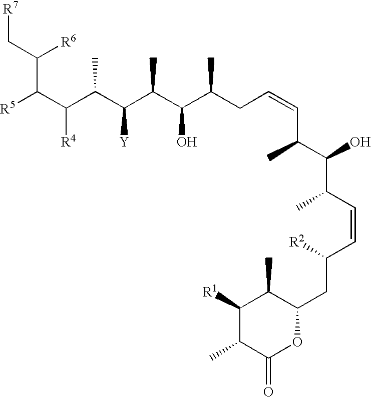

wherein

R¹ and R² are each independently hydrogen, hydroxyl, or a hydroxyl protecting group;

R⁴, R⁵, R⁶, and R⁷ are each hydrogen, or R4 and R⁵ together form a double bond and R⁶ and R⁷ together form a double bond; and,

Y is hydroxyl, amino, —OC(═O)NH₂ or —NHC(═O)NH₂, provided that at least one of R¹ and R² is not hydroxyl.

In yet another embodiment of the present invention, compounds are provided of the formula

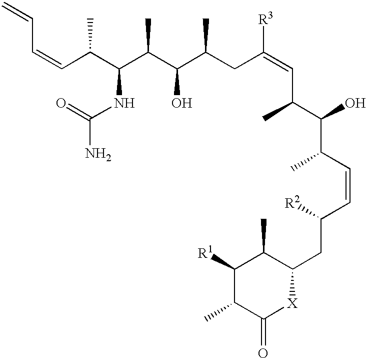

wherein

R¹ and R² are each independently hydrogen, hydroxyl, or a hydroxyl protecting group;

R³ is hydrogen, C₃-C₁₀ alkyl or aryl;

X is O, NH, or N-alkyl; and,

Y is hydroxyl, amino, —OC(═O)NH₂ or —NHC(═O)NH₂,

Particularly preferred embodiments of the present invention include but are not limited to:

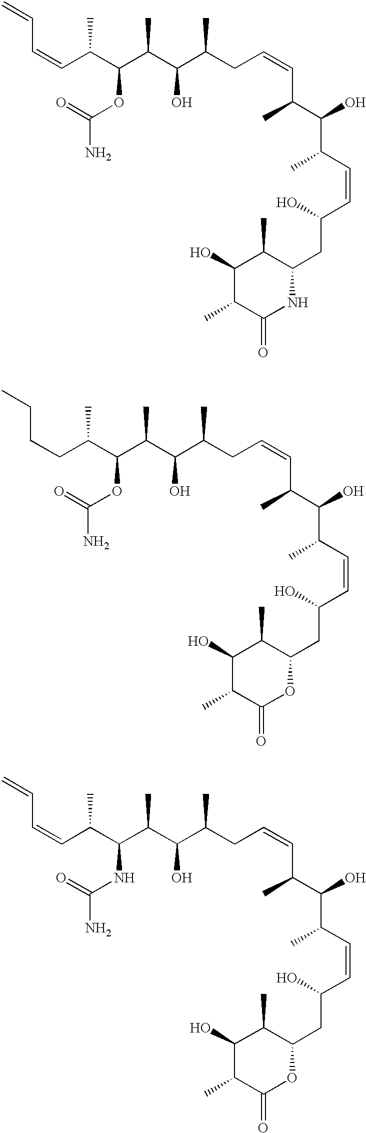

In another aspect of the invention, intermediates leading to the synthesis of the above discodermolide analogs are provided.

In one embodiment, intermediates of the formula

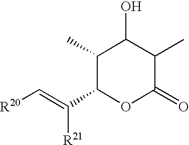

are provided, wherein R²⁰ is hydrogen, alkyl, or aryl; and R²¹ is hydrogen or alkyl. Preferred embodiments include but are not limited to:

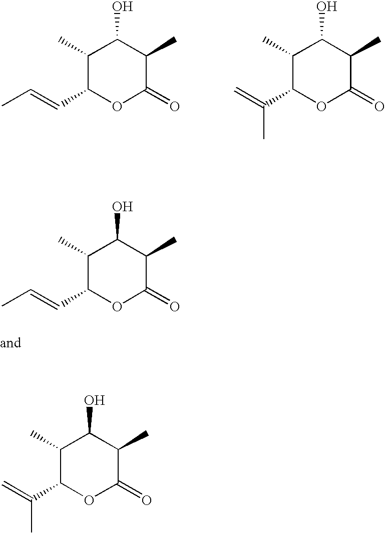

In another embodiment, compounds of the formula

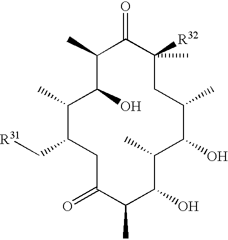

are provided, wherein

R³¹ is hydrogen, alkyl, alkenyl, halogen or phenylthio; and

R³² is hydrogen or hydroxy;

with the proviso that when R³¹ is hydrogen or alkyl, that R³² can not be hydrogen.

Preferred embodiments include but are not limited to:

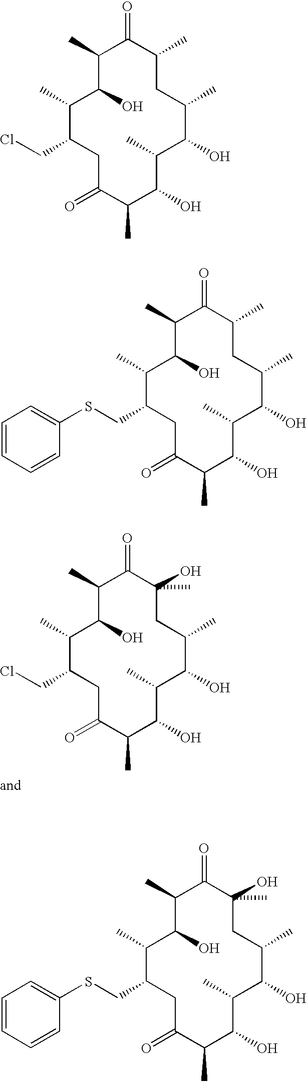

Genes and Enzymes of the Invention

In one aspect of the invention, novel polyketide synthase genes are constructed by combining fragments of naturally occurring PKS genes, along with a DNA sequence encoding a terminal thioesterase domain located at the end of the sequence encoding the last extender module, and cloning them into suitable expression vectors behind functional promoters. Examples of suitable expression vectors for actinomycete host cells, such as Streptomyces, include both autonomously replicating vectors and integrating vectors which insert into the host chromosome. Preferred examples of replicating vectors for Streptomyces include those based on the SCP2* replicon, such as pRM1 and pRM5. Preferred examples of integrating vectors include but are not limited to vectors containing sequences allowing for integration at phage attachment sites. Particularly preferred examples of integrating vectors for Streptomyces are those using the φC31 phage sequences, including but not limited to pSET and pSAM. A listing of suitable actinomycete vectors is found in Kieser et al, “Practical Streptomyces Genetics,” (John Innes, Norwich, 2000), which is incorporated herein by reference.

Expression of the constructed PKS genes in a suitable host results in production of a functional PKS. Suitable actinomycete host cells include but are not limited to members of the genera Streptomyces, Saccharopolyspora, and Micromonospora. Preferred examples of actinomycete host cells are members of the genera Streptomyces and Saccharopolyspora. Particularly preferred actinomycete host cells are Streptomyces coelicolor, Streptomyces lividans, Streptomyces fradiae, and Saccharopolyspora erythraea. Suitable host cells typically have had their native PKS genes deleted or otherwise rendered non-functional, for example through mutagenesis, according to the methods described in Khosla et al., “Recombinant production of novel polyketides” U.S. Pat. No.5,830,750 (incorporated herein by reference). Particularly preferred examples of non-actinomycete host cells include suitably prepared Escherichia coli, Saccharomyces cerevesiae, and Myxococcus xanthus. The preparation and use of Escherichia coli and Saccharomyces cerevesiae host cells is described in Santi et al., “Heterologous production of polyketides,” PCT publication no. WO01/31035 and in Pfeifer & Khosla, “Biosynthesis of polyketide substrates,” PCT publication no. WO01/27306 (both of which are incorporated herein by reference). The use of Myxococcus xanthus as a host cell is described in Julien et al., “Producing epothilone and epothilone derivatives,” PCT publication no. WO/00/31247 (incorporated herein by reference).

In one embodiment, n-module PKS genes are constructed by fusing contiguous coding sequences for modules from a natural PKS to a coding sequence for terminal TE domain to produce a new PKS gene. Such novel PKS enzymes and the genes that encode them are herein designated:

(source)[module(1)]-[module(2)]-. . . -[module(n-1)]-[module(n)]-TE.

As an example, a two-module PKS comprising modules 5 and 6 of the erythromycin PKS genes along with the TE domain is herein designated:

(ery)[module(5)]-[module(6)]-TE.

In another embodiment, PKSs comprising modules are constructed by fusing modules or contiguous sets of modules obtained from different natural PKS genes. As an example, a three-module PKS comprising module 1 of the erythromycin PKS fused with modules 5 and 6 of the narbomycin PKS and a TE is herein designated:

(ery)[module(1)]-(nar)[module(5)]-[module(6)]-TE.

In another embodiment of the invention, PKSs comprising modules which have been mutated so as to alter the complement of domains contained within the modules are constructed. Such PKSs are constructed either using modules from the same or from different natural PKS genes. Domains which have been inactivated through mutagenesis, but not deleted, are indicated by the symbol “°.” As an example, a three-module PKS comprising module 1 of the erythromycin PKS, wherein the KS domain has been inactivated through mutagenesis, fused with modules 5 and 6 of the narbomycin PKS and a TE is herein designated:

(ery)[module(1)-KS°]-(nar)[module(5)]-[module(6)]-TE.

Domain deletions are indicated by “Δ,” such that a two-module PKS comprising modules 1 and 2 of the erythromycin PKS, in which the KR domain of module 2 has been deleted, is herein designated:

(ery)[module(1)]-[module(2)-ΔKR]-TE.

Domain substitutions are indicated by a “/.” Thus, a two-module PKS comprising modules 1 and 2 of the erythromycin PKS, in which the KR domain of module 2 has been replaced with the KR domain taken from module 4 of the rapamycin PKS, is herein designated:

(ery)[module(1)]-[module(2)KR/rapKR4]-TE.

Domain additions are indicated by “+,” such that a two-module PKS comprising modules 1 and 2 of the erythromycin PKS, in which the MT domain from module 8 of the epothilone PKS gene has been added without other alteration of the module domains, is herein designated:

(ery)[module(1)]-[module(2)+epoMT8]-TE.

Examination of polyketide structures reveals multiple occurrences of modules having identical functions in various PKSs. Because of this, several PKS genes may provide modules of equivalent function, and may be used interchangeably according to the present invention. Thus, while specific sources for modules are used in the description of the invention for the purposes of illustration, it is intended that modules of equivalent function from different sources may be freely interchanged in accord with the methods of the invention.

### BRIEF DESCRIPTION OF THE DRAWINGS

FIG. 1 depicts the organization of the eryAI, eryAII, and eryAIII genes that encode the PKS enzyme deoxyerythronolide B synthase (“DEBS”) (which is composed of DEBS1, DEBS2, and DEBS3 protein subunits) that makes 6-deoxyerythronolide B (“6-dEB”).

FIG. 2 is the macrolactonization synthetic strategy developed by Danishefsky for the de novo synthesis of epothilone D starting from two key intermediates, a thiazole fragment and Compound A.

FIG. 3 depicts the organization of a two-module PKS capable of converting Compound (2) into Compound (1).

FIG. 4 is a graphical representation of the epothilone PKS.

FIG. 5 illustrates the relationships between some key compounds of the invention. Single arrows indicate biological or biochemical transformations, while double arrows indicate chemical transformations. Arrows may represent multiple steps.

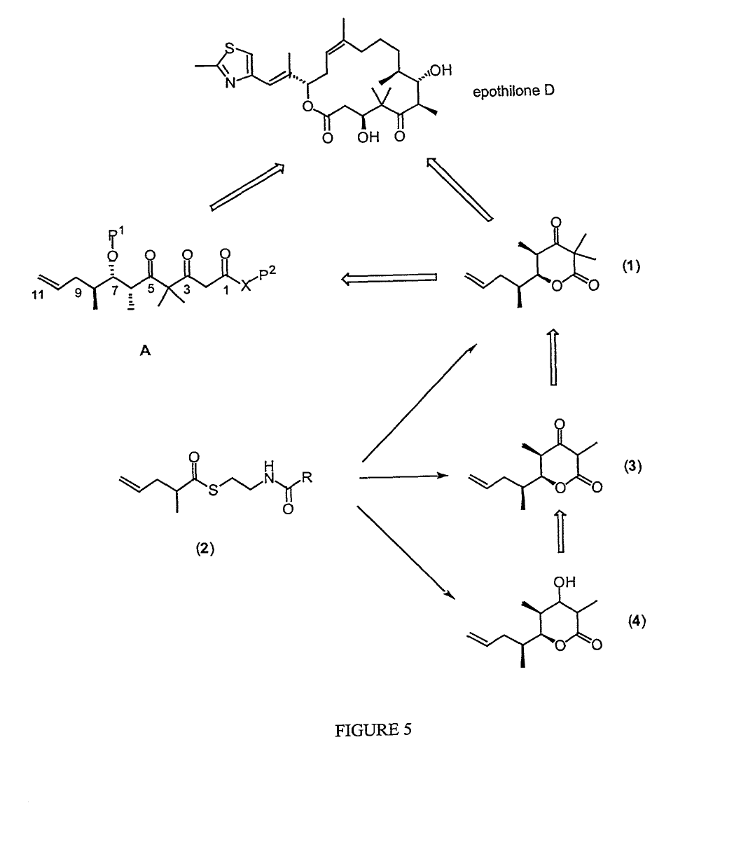

FIG. 6 illustrates a novel protocol for the synthesis of epothilone D using Compound (10) in place of Compound A.

FIG. 7 illustrates another novel protocol for the synthesis of epothilone D using Compound (1) in place of Compound A.

FIG. 8 shows the polyketide structures produced by 14 common PKS modules, along with reported cases of modules with MT domains.

### DETAILED DESCRIPTION OF THE PREFERRED EMBODIMENTS

Polyketides are naturally occurring compounds that are made by many organisms including fungi and mycelia bacteria. A diverse class of natural products, polyketides are classified as such because they are synthesized, at least in part, from two carbon unit building blocks through a series of Claisen type condensations by polyketide synthase (“PKS”) enzymes. Two major types of PKS enzymes are modular PKS and iterative (or “aromatic”) PKS. Modular PKS enzymes are typically multi-protein complexes in which each protein contains multiple active sites, each of which is used only once during carbon chain assembly and modification. Iterative PKS enzymes are typically multi-protein complexes in which each protein contains only one or at most two active sites, each of which is used multiple times during carbon chain assembly and modification.

Polyketides made by modular PKS enzymes have a variety of biological activities and include important drugs such as erythromycin and tacrolimus (also known as FK-506). A prototypical example of modular PKS enzymes is deoxyerythronolide B synthase (“DEBS”) that synthesizes 6-deoxyerythronolide B (“6-dEB”), an erythromycin precursor. The organization of these eryA genes which encode DEBS and/or methods for their manipulation are described in U.S. Pat. Nos. 5,712,146 and 5,824,513, 6,004,787, 6,060,234, and 6,063,561 each of which is incorporated herein by reference.

Modular PKS enzymes are so termed because they are organized into distinct units (or modules) that ultimately control the structure of a discrete two-carbon portion of the polyketide the structure. PKS enzymes generally contain (i) a loading domain, (ii) a number of extender modules, (iii) and a releasing domain (which is also called a thioesterase domain). The two-carbon units are of the general formula (R—C(═O)) from which polyketides are synthesized and are generally referred to as starter units or extender units depending on whether the two carbon unit initiates the synthesis of the polyketide or extends (adds to) the growing polyketide chain during synthesis. Starter units bind to the loading domain and initiate the polyketide synthesis and (ii) extenders bind to the extender modules and extend the polyketide chain. Starter units and extender units are typically acylthioesters, most commonly acetyl-CoA, propionyl-CoA, and the like for starter units and malonyl-CoA, methylmalonyl-CoA, methoxymalonyl-CoA, hydroxymalonyl-CoA, ethylmalonyl-CoA, and the like for extender units.

Each module of a modular PKS contains three core domains needed for polyketide synthesis, an acyltransferase (AT) responsible for selecting and binding the appropriate extender unit, an acyl-carrier protein (ACP) responsible for carrying the growing polyketide chain, and a β-ketoacylsynthase (KS) responsible for condensing the extender unit onto the growing polyketide chain. Together, these core domains add a 2-carbon β-ketothioester onto the growing end of the polyketide chain.

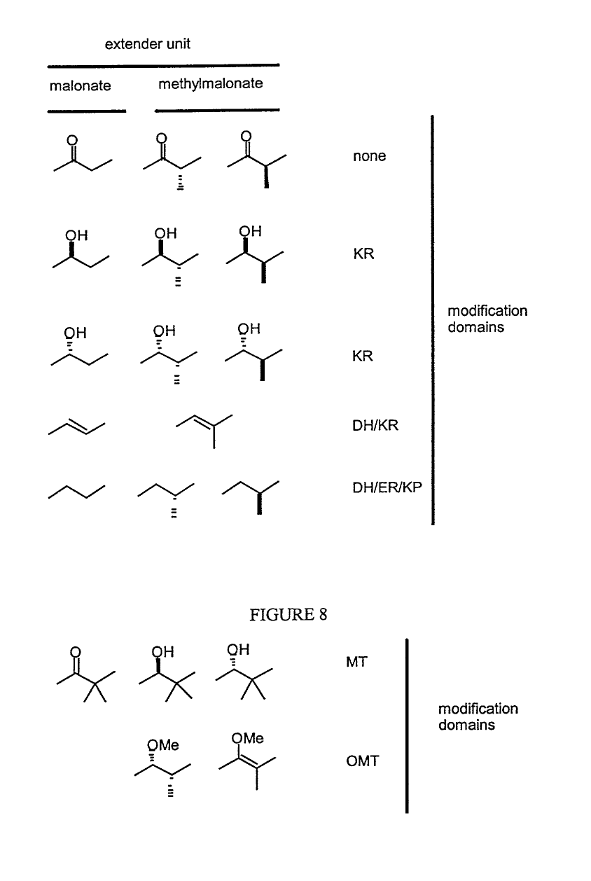

In addition, a module may contain a set of reductive cycle domains responsible for modifying the β-ketone produced by the core domains. If present, a ketoreductase (KR) domain reduces the β-ketone to an alcohol of defined stereochemistry. If present with a KR, a dehydratase (DH) domain eliminates the alcohol produced by the KR to form an alkene. If present with a DH and a KR, an enoylreductase (ER) domain reduces the alkene produced by the DH to form a saturated alkane. Other types of modification domains, such as methyltransferase (MT) domains, can also be present in a module. MT domains add a methyl group, typically from S-adenosylmethionine (SAM), to the α-carbon of the newly-added 2-carbon unit, and O-methyltransferase domains (OMT) add the methyl group instead to the oxygen atom of the enol form of the β-ketothioester to form a methyl vinyl ether or to the alcohol resulting from the action of a KR domain so as to form a methyl ether. Examples of known modules with MT or OMT domains are shown in FIG. 8.

A listing of the 14 functional modules possible using only malonyl- and methylmalonyl-specific AT domains and only KR, DH, and ER modification domains is given in FIG. 8. Other modules are known, as AT specificities for ethylmalonyl- and methoxymalonyl-extender units are also known.

The order of modules as they are encoded within the gene appears to follow the order in which they function in the biosynthesis. The order of domains within a module, while conserved between PKSs, does not appear to follow the order in which they function in the biosynthesis. The boundaries of domains are readily recognized due to the high homology that exists between the many known examples of PKS genes.

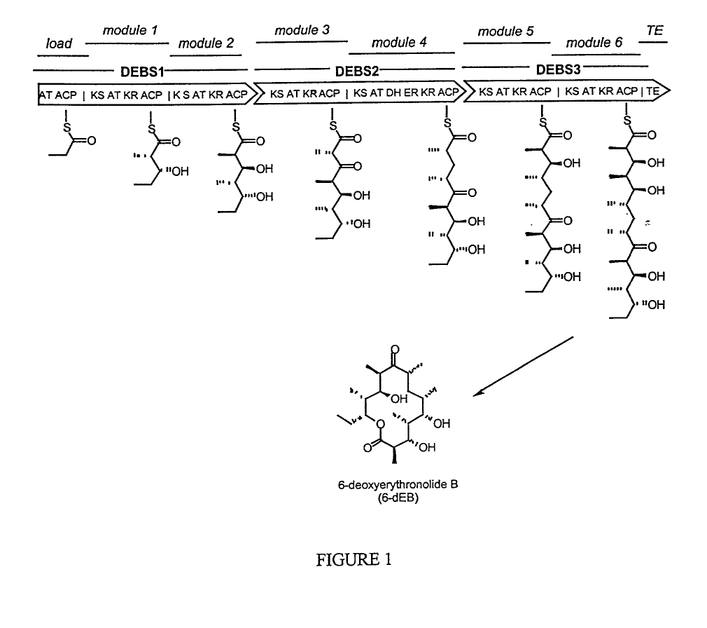

FIG. 1 is a schematic representation of the DEBS enzyme, which is responsible for the biosynthesis of the polyketide core of erythromycin. DEBS is the prototypical modular PKS, and is a dimer of three proteins, DEBS1, DEBS2, and DEBS3. The organization of the genes that encode DEBS and methods for their manipulation are described in U.S. Pat. Nos. 5,712,146, 5,824,513, 6,004,787, 6,060,234, and 6,063,561, each of which is incorporated herein by reference.

As shown by FIG. 1, DEBS1 comprises the loading domain and the first and second extender modules. DEBS2 comprises the third and fourth extender modules. DEBS3 comprises the fifth and sixth extender modules and the releasing domain. Modular PKSs are commonly dimers of multiple polypeptides, although the division of number of modules between the polypeptides is highly variable.

The DEBS loading domain consists of a special AT domain that binds the starter unit and an acyl carrier protein (“ACP”). Synthesis of 6-dEB progresses as follows. The loading domain AT recognizes propionyl CoA and binds the acyl group (the propionyl group) though a serine residue (Ser—O—C(═O)—CH₂CH₃). The loading domain AT transfers the acyl group to the loading domain ACP which binds the acyl group through a cysteine residue (Cys—S—C(═O)—CH₂CH₃). Concurrently, each of the six extender modules recognizes methylmalonyl CoA and transfers the corresponding acyl group to the ACP of that module (Cys—S—C(═O)—CH (CH₃)C(═O)OH). Synthesis commences when the loading domain ACP transfers the propionyl group to the KS of the first extender module which positions the group for the condensation reaction with the methylmalonyl thioester of the first extender module ACP (with a concomitant elimination of CO₂). After the condensation reaction, the first extender ACP now has bound a β-ketothioester (—Cys—S—C(═O)—CH(CH₃)C(═O)CH₂CH₃ where the italicized portion represents the two carbon unit from the methyl malonyl CoA and the bold portion represents the two carbon unit from the propionyl CoA).

Because of the presence of the KR domain in module 1, the keto group of the β-ketothioester is modified into an alcohol. This modified precursor is depicted bound to the first extender module ACP in FIG. 1. As the synthesis progress, this precursor is transferred to second extender module KS where the recently-extended acyl group is positioned for the condensation reaction with the methylmalonyl thioester bound to the second extender module ACP. The polyketide chain attached to the second extender module ACP also shows the previous keto group reduced to an alcohol. Because the third extender module KR domain is inactive, the polyketide chain attached to the third extender module ACP shows an unmodified keto group. The polyketide chain attached to the fourth extender module shows a fully saturated two-carbon unit due to the presence of a KR, DH and ER domains. As with the first and second extender module ACPs, the polyketide chains attached to the fifth and sixth extender modules depict an alcohol moiety due to the presence of the respective KR domains. Finally, the polyketide synthesis terminates when it is released from the PKS enzyme by the TE domain and forms a cyclic ester (also called a lactone or macrolactone).

Other modular PKS enzymes synthesize polyketides in a similar manner. The variations in structure of naturally occurring polyketides result from differences in the identities of the starter unit, the number of extender modules, differences in the identity of the extender unit that is recognized by each extender module, and the presence or absence of additional functionalites (e.g., KR, DH, ER, MT) in each extender module. The same type of variations can be engineered using recombinant techniques to produce derivatives of naturally occurring polyketides and entirely novel polyketides. Recombinant methods for manipulating modular PKS genes are described, for example, by U.S. Pat. Nos. 5,672,491; 5,712,146; 5,830,750; and 5,843,718; and in PCT patent publication Nos. 98/49315 and 97/02358, each of which is incorporated herein by reference.

The present invention takes advantage of these techniques to provide a general process for making any polyketide that is independent of a host organism or a naturally occurring PKS gene. The method generally comprises:

Comparing the structure of a polyketide or polyketide-like compound to be made biologically with a library of PKS structures;

identifying at least one library structure having a common element with the structure of said compound;

associating a PKS gene for each identified library structure; and,

designing a new gene capable of producing said compound wherein the new gene includes at least a portion of one PKS gene corresponding to an identified library structure. A method for implementing such a method using a computer has been previously described in PCT Patent Application No. US01/17352 filed on May 29, 2001 entitled Design of Polyketide Synthase Genes by inventors Daniel V. Santi et al., which is incorporated herein by reference.

In preferred embodiments, the method further involves dividing the target polyketide structure into two-carbon units and for each two-carbon unit, identifying an extender module that would provide the corresponding structure. The method comprises:

describing the target compound as sequence of two-carbon units;

comparing the two-carbon unit sequence of the target compound with a database of naturally occurring PKS structures wherein each database PKS structure is also described as a sequence of two-carbon units;

for each two-carbon unit of the target compound, searching the database for a matching two-carbon unit;

for each two-carbon unit of the target compound for which a match was found in the database, associating a PKS gene fragment corresponding to the matched database two-carbon unit; and,

designing a new gene capable of producing said compound wherein the gene includes the PKS gene fragments associated with the matched database two-carbon units.

Ideally, each two-carbon unit of the target compound will find matching counterparts in the library or database of naturally occurring polyketides. When this is the case, the identified PKS gene fragments or extender modules are then assembled together with a releasing domain into a gene which when expressed as a functional PKS system will make the desired product. The extender modules may be from a single PKS enzyme or multiple PKS enzymes although it is generally preferred to maximize the number of consecutive modules that are taken from a single PKS enzyme. In this manner, genes are constructed that have the minimum number of non-native module boundaries to avoid undue disruption to the PKS enzyme structure.

For simplicity the general method will be illustrated for a polyketide fragment having the following structure

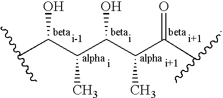

The fragment includes two two-carbon units, i and i+1. The i-th extender module attaches the two carbon unit whose backbone carbons are designated as alphaᵢ and betaᵢ and the second extender module attaches the two carbon unit whose backbone carbons are designated as alphaᵢ₊₁ and betaᵢ₊₁.

The components that are required for extender modules i and i+1 are analyzed by examining the groups off of the alpha and beta carbons. As described previously, differences in the substituents off the carbons at the alpha positions are due to the differing extender module AT specificities for acylthioesters. Two groups that are commonly found as substituents off the alpha carbon are methyl and hydrogen which are due to the extender module AT's specificity for methylmalonyl CoA and malonyl CoA respectively. Similarly, other groups include ethyl, hydroxy, and methoxy which generally are due to the extender module AT's specificity for ethylmalonyl CoA, hydroxymalonyl CoA, and methoxymalonyl CoA respectively. The differences in the functionality associated with the beta carbons are due to absence or the presence of one or more modification domains as described above.

In the above fragment, the i−th extender module requires an AT that is specific for methyl malonyl CoA (due to the methyl group off alphaᵢ) and a KR due to the presence of an alcohol moiety off the betaᵢ₋₁ carbon. Consequently, the i−th extender module would comprise KS, AT, KR, and ACP domains. The i+1 extender module also requires an AT that is specific for methyl malonyl CoA (due to the methyl group off alphaᵢ₊1). Because of the hydroxyl group off betaᵢ, the i+1 extender module also would comprise KS, AT, KR and ACP domains. The keto group off the betaᵢ₊₁ carbon indicates that the next extender module (or the i+2 extender module) will not require any additional functionality enzymes. In other words, the i+2 extender module would only comprise KS, AT, and ACP domains. A module consisting of KS, AT, and ACP domains is termed a minimal module.

Many different strategies can be used to make the portion of a gene that can synthesize the above fragment. In one embodiment, extender modules from known PKS genes may be used. For example, the structure of the above fragment is identical to the portion of the erythromycin that is synthesized by extender modules 5 and 6 of the erythromycin PKS gene. See e.g., FIG. 1. As a result, the gene that encodes for extender modules 5 and 6 may be used without modification.

Exact matches of a particular fragment structure to consecutive modules of a single PKS are more likely where the number of two-carbon units that comprise the fragment is small. If such an exact match were not found, then a gene for making the desired fragment may be constructed in accordance with the methods of the invention from multiple PKS genes. For example, if a desired fragment needs to be constructed with six extender modules, it may be constructed by combining two extender modules from a first PKS and four modules from a second PKS. Although the genes that encode the modules are manipulated to allow the modules from the two different PKSs work together, the individual module's AT specificity or the number of functionality enzyme domains is not usually altered. Where possible, it is generally preferred to use the maximum number of consecutive modules from a single PKS gene. In other words, genes are constructed that have the minimum number of non-native module boundaries to avoid undue disruption to the resulting PKS enzyme structure as well as minimize the number of cloning manipulations that must be performed in order to assemble the genes.

Because module organization may be inferred from the final polyketide structure, practice of the present invention is amenable even in situations where the component PKS gene itself has not yet been identified. For example, if the DEBS gene were not known, the fact that the desired fragment is identical to the portion of 6-dEB that is encoded by extender modules 5 and 6 of the DEBS PKS gene may still be determined from the general organization of modular PKS genes. If the portion of the DEBS gene is determined to be the most suitable, then a probe may be constructed from conserved regions of known PKS genes to find and sequence the DEBS gene. In general, intact PKS genes are readily retrievable because the genes coding for the core components of the PKS (loading domain, extender modules, and releasing domain) as well as the genes for the tailoring enzymes are generally contiguous. Once the desired PKS gene is obtained, its organization may be determined and the coding sequences for its modules used as described herein.

In another embodiment, the required extender modules are engineered by modifying one or more modules of a particular PKS. An illustrative example of a modification is changing the AT specificity of a module. Another modification is changing the number of functionality domains by either adding or deleting one or more functionality domains. In a variation of the latter, the function of an existing functionality domain may be inactivated. In each of these cases, the method comprises

describing the compound as sequence of two-carbon units;

comparing the two-carbon unit sequence of the compound with a database of naturally occurring PKS structures wherein each database PKS structure is also described as a sequence of two-carbon units;

identifying a database PKS structure having the most number of matching consecutive two-carbon units and having at least one non-matching two-carbon unit;

identifying a PKS gene responsible for making the database PKS structure;

determining one or more alterations to the PKS gene in the portion responsible for making the at least one non-matching two-carbon unit to make a matching two-carbon unit.

An illustration of this embodiment also benefits from a simplified example. Using the prototypical PKS gene, extender modules 5 and 6 of the DEBS PKS are modified to correspond to the following fragment structure:

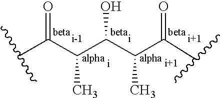

This fragment differs from the fragment used in an earlier example in that there is a keto instead of a hydroxyl group off the betaᵢ₋₁ carbon. As a result, a gene that would correspond to the fragment would include the following sequence: KSᵢ, ATᵢ, ACPᵢ₊₁, KSᵢ₊₁, ATᵢ₊₁, KRᵢ₊₁ and ACPᵢ₊₁ wherein both ATᵢ and ATᵢ₊₁ possess specificities for methyl malonyl CoA. The sequence of domains for extender modules 5 and 6 of the erythromycin PKS is: KS₅, AT₅, KR₅, ACP₅, KS₆, AT₆, KR₆ and ACP₆. As a result, the sequence of domains corresponding to erythromycin PKS modules 5 and 6 needs to be modified where the function of KR₅ is inactivated. The inactivation may occur by mutating the sequence of KR₅ so that it is no longer functional or by deleting the domain all together. Although the above example is simplistic, it serves as a platform to illustrate that an existing module may be modified in a number of ways. First, its AT may be replaced with another AT having a different specificity (a malonyl CoA specific AT for a methyl malonyl CoA specific AT) or an existing AT may be mutated to possess a different specificity. Second, existing functionality domains may be inactivated as the KR₅ in the above example. Alternatively, functionality domains may be added. For example, a KR may be added to a minimal module comprising KS, AT, and ACP. Similarly, a DH or a DH and an ER may be added to a domain comprising KS, AT, KR, and ACP

Due to the simplicity of this example, the novel gene was designed using components derived from a single PKS gene. For more complicated compounds, the novel PKS gene will likely be a chimeric gene made from at least two PKS genes that each encode a naturally occurring PKS compound. Many such PKS genes are known and are suitable for this purpose, including but not limited to the PKSs involved in the biosynthesis of erythromycin, megalomicin, picromycin, narbomycin, oleandomycin, lankamycin, FK506, FK520 (ascomycin), rapamycin, epothilone, tylosin, spiramycin, rosamicin, geldanamycin, pimaricin, FR008, candicidin, avermectin, and the like. Further, methods for identification and cloning of new PKS genes are known, as for example in Santi et al., “Method for Cloning PKS Genes,” PCT publication WO01/53533, published Jul. 26, 2001 (incorporated herein by reference), and can be used to obtain fragments of PKS genes to be used according to the methods of the present invention.

According to the methods of the invention, the above described methods may be reiterated any number of times to synthesize any polyketide. The polyketide may be the final compound or may be used as a starting material for further chemical modification. Although the present invention may be used to make novel polyketides, it may also be used to make known polyketides using novel PKS genes. For example, epothilone may be made with a chimeric gene in spite of the fact that the epothilone gene is known. However, because epothilone is naturally produced in such low yields, strategies involving chimeric genes, particularly those comprising high expressing PKS genes, may also result in high expression of the chimeric epothilone producing genes.

Epothilone

A variation of making full length polyketides is making polyketide fragments that may be used as stereochemically pure reagents in chemical syntheses. Unlike non-enzymatic reactions typical of synthetic organic chemistry, enzymatic reactions typically show essentially absolute stereoselectivity. Thus, the complex sequence of reactions performed by DEBS (ca. 27 steps) proceed with apparent absolute stereocontrol, as no stereoisomers of the product 6-deoxyerythronolide B have been identified from the enzymatic reaction.

In one embodiment of this aspect of the present invention, novel compounds and methods are used to make a previously described compound, as illustrated herein with epothilone intermediates. In other aspects of the present invention, novel compounds and methods are used to make novel intermediates for use in previously described synthetic protocols, as illustrated herein with reference to epothilones. In yet other aspects of the present invention, novel compounds and methods are used to make a compound using novel synthetic strategies as illustrated herein with reference to epothilones. As described previously, an obstacle in the clinical evaluation of epothilones is the limited quantities that may be obtained from natural sources. Although several groups have developed de novo synthetic protocols for making epothilones A-D, these syntheses are complex, cumbersome, and not generally suitable for making large quantities of material. A general problem with these protocols is the difficulty in synthesizing certain precursor compounds typically due to the number and complexity of the compounds' stereocenters. In many cases, if the syntheses of these precursors were simplified, the de novo protocols for making polyketides such as epothilone on a commercial scale would become economically feasible.

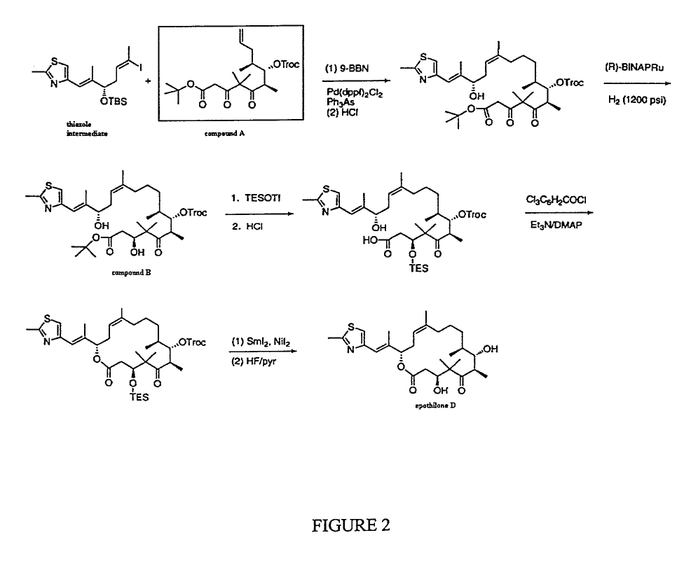

An illustrative de novo synthetic protocol for making epothilone is the macrolactonization strategy outlined by Danishefsky and coworkers (Balog et al., 1998, A novel aldol condensation with 2-methyl-4-pentenal and its application to an improved total synthesis of epothilone B, Angew. Chem. Int. Ed. Engl. 37(19): 2675-2678, incorporated herein by reference). FIG. 2 illustrates the synthetic protocol as it is applied to the synthesis of epothilone D starting from two key intermediates, the thiazole intermediate and compound A.

The formula of compound A is

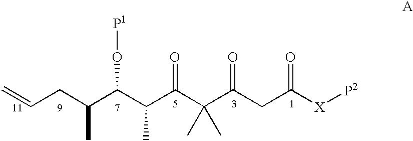

wherein P¹ and P² are H or protecting groups and X is either oxygen or sulfur. In the Danishefsky protocol, P¹ is 2,2,2-trichloroethoxycarbonyl (“Troc”), p² is tert-butyl, and X is oxygen. The C10-C11 double bond is coupled to the thiazole intermediate via Suzuki coupling. The carboxylate group at C1 is subsequently used to form the epothilone macrolactone via an intramolecular esterification. As shown by FIG. 2, prior to the macrolactonization reaction, a Noyori reduction is performed to reduce the C-3 keto group derived from Compound A into an alcohol to provide the appropriate epothilone functionalities off carbons 1 through 11.

If Compound A, or an advanced precursor of Compound A comprising the stereogenic centers in Compound A, could be made biologically, many of the stereoselectivity problems associated with making epothilone would be eliminated, allowing the cost-effective synthesis of the epothilones.

In one aspect of the invention, a compound (1) of the following formula

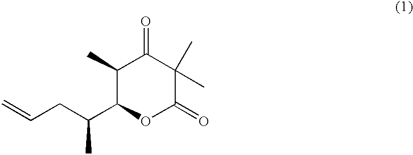

comprising C3-C11 of Compound A is made using an engineered polyketide synthase. In one embodiment, (1) is converted through a short series of chemical transformations into Compound A. In a further embodiment, (1) is converted more directly into epothilone.

In one embodiment of the invention, (1) is prepared by providing racemic 2-methyl-4-pentenoate N-acetylcysteamine thioester (2), a compound of the following formula

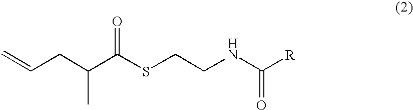

to an engineering polyketide synthase (PKS) capable of converting (2) into (1). A detailed protocol for making (2) is found in Example 1. General methods for making various thioesters and using the same for making polyketides are disclosed for example by U.S. Pat. Nos. 6,066,721 and 6,080,555 and PCT publication WO 99/03986 which are all incorporated herein by reference. Briefly, the thioester mimics a nascent polyketide chain and thus feeds into the polyketide synthetic process starting from the first extender module of the functional PKS system.

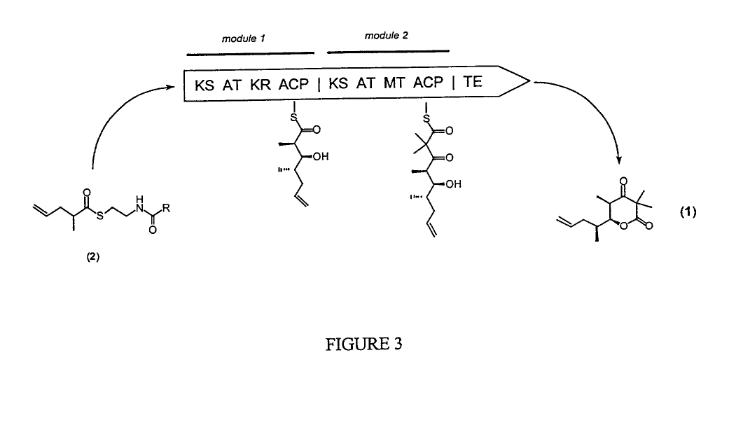

A two-module PKS containing the appropriate domains will convert (2) into (1). The first module recognizes (2) as the starter unit, then adds a methylmalonyl extender unit and reduces the resulting β-ketone unit to an (S)-alcohol. The second module adds a methylmalonyl extender unit and a methyl group. Finally, a thioesterase domain produces the lactone and releases (1) from the PKS. This process is illustrated in FIG. 3.

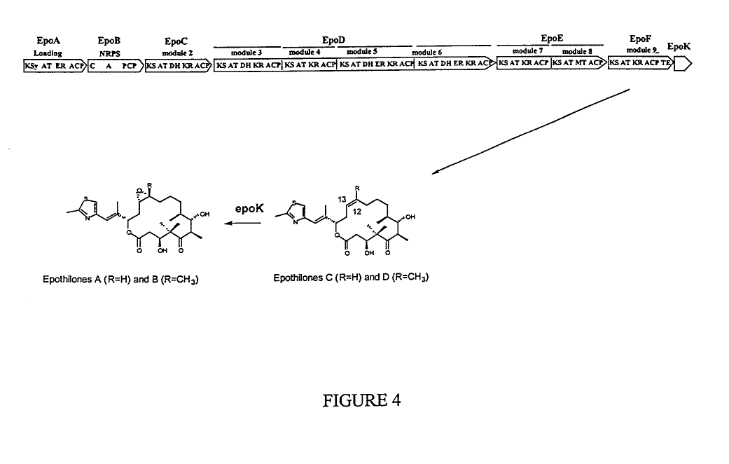

One embodiment of the invention provides a two-module fragment of a naturally-occurring PKS comprising all the above-listed activities required for conversion of (2) into (1). An example of such a fragment is modules 7 and 8 of the epothilone PKS (FIG. 4), fused with a thioesterase (TE) domain so as to produce the PKS (epo)[module(7)]-[module(8)]-TE. The genes for the epothilone PKS are described in Tang et al., “Recombinant methods and materials for producing epothilone and epothilone derivatives,” PCT publication no. WO 00/31247 (incorporated herein by reference). The TE domain is taken either from the epothilone genes or from a heterologous gene, for example the DEBS genes. Methods for the construction and heterologous expression of two-module fragments of PKSs having fused TE domains are described in Khosla et al., “Production of novel polyketides,” U.S. Pat. No.5,712,146 (incorporated herein by reference). Compound (1) is produced according to the method of the invention when a growing culture of an organism, for example Streptomyces coelicolor CH999, containing an expression system for modules 7 and 8 of the epothilone PKS, is supplied with (2). The resulting (1) is extracted from the culture medium according to methods known in the art. Other heterologous expression hosts, including but not limited to Streptomyces lividans, Myxococcus xanthus, Escherichia coli, and Saccharomyces cerevesiae, may be used as described in Barr et al., “Production of polyketides in bacteria and yeasts,” U.S. Pat. Nos. 6,033,883 and 6,258,566, and Tang et al., “Recombinant methods and materials for producing epothilone and epothilone derivatives,” PCT publication No. WO/0031247 (each of which is incorporated herein by reference).

Another embodiment of the invention provides a two-module PKS for the conversion of (2) into (1) resulting from the genetic engineering of a two-module fragment taken from a naturally-occurring PKS. An example of such a fragment is modules 5 and 6 of the narbonolide PKS from either Streptomyces venezuelae or Streptomyces narbonensis, along with the natural TE domain, genetically engineered so as to incorporate a methyltransferase domain in module 6 so as to produce the PKS (nar)[module(5)]-[module(6)+epoMT8]-TE (FIG. 3). The genes for the narbonolide PKS and their heterologous expression have been described in McDaniel et al., U.S Pat. No.6,117,659 (incorporated herein by reference). Suitable methyltransferase domains have also been described, for example from module 8 of the epothilone PKS. Methods for manipulating PKS domains by domain addition and replacement are described in, for example, McDaniel, “Library of Novel “Unnatural” Natural Products, PCT publication WO/0024907 (incorporated herein by reference).

Compound (1) is produced according to the method of the invention when a growing culture of an organism, for example Streptomyces coelicolor CH999, containing an expression system for (nar)[module(5)]-[module(6)+epoMT8]-TE, is supplied with (2). The resulting (1) is extracted from the culture medium according to methods known in the art.

As a further example, an engineered form of the DEBS3 protein of the erythromycin PKS is used. The DEBS3 protein is one of three protein subunits in the erythromycin PKS, and includes extender module 5, extender module 6, and the terminating thioesterase. Both modules naturally contain active KR domains. In this embodiment, DEBS3 is mutated so as to inactivate the ketoreductase domain of module 6 and incorporate a methyltransferase domain, so as to produce (ery)[module(5)]-[module(6)-KR°+epoMT8]-TE. In an alternate example, the KR domain is deleted, giving rise to (ery)[module(5)]-[module(6)-ΔKR+epoMT8]-TE. These PKS genes are heterologously expressed in a host such as Streptomyces coelicolor CH999, Streptomyces lividans, Escherichia coli, or Saccharomyces cerevesiae.

Two-module PKSs derived from natural PKSs other than DEBS are useful to convert (2) into (1). Modules 5 and 6 from the megalomicin PKS may also be used, for example. Recombinant methods for manipulating modular PKS genes are described, for example, by U.S. Pat. Nos. 5,672,491; 5,712,146; 5,830,750; and 5,843,718; and in PCT publication Nos. 98/49315 and 97/02358, each of which is incorporated herein by reference.

In another embodiment of the invention, a PKS for the conversion of (2) into (1) resulting from the combination of two or more modules taken from different naturally-occurring PKSs is provided. Thus, a module from DEBS and a module from the epothilone PKS may be combined. As an example, the gene (ery)[module(5)]-(epo)[module(8)]-TE is constructed and expressed in Streptomyces coelicolor CH999. When supplied with (2), the resulting PKS produces (1). Methods for the construction and optimization of hybrid-modules PKSs are described in Tang et al., “Formation of functional heterologous complexes using subunits from the picromycin, erythromycin, and oleandomycin polyketide synthases,” Chemistry & Biology (2000), 7:77:84 ; and Gokhale et al., “Methods to mediate PKS module effectiveness,” PCT publication WO/0047724 (both of which are incorporated herein by reference).

In yet another embodiment of the invention, a PKS containing more than two modules is provided for the conversion of Compound (2) into Compound (1), in which the first module serves as a loading module for Compound (2) and is incapable of adding an extender unit onto the polyketide chain. An example is a three-module PKS comprised of modules 1, 2, and 3 of DEBS, in which the KS of module 1 has been inactivated through, for example, mutagenesis, and a methyltransferase domain has been added to module 3, along with the terminal thioesterase domain, so as to provide (ery)[module(1)-KS°]-[module(2)]-[module(3)+epoMT8]-TE. Engineering of the three-module PKS containing modules 1, 2, and 3 of DEBS has been described in McDaniel et al., “Gain-of-function mutagenesis of a modular polyketide synthase,” J. Am. Chem. Soc. (1997) 119:4309-4310, (incorporated herein by reference). Construction of the [KS°] mutant is done either by mutagenesis of the three-module construct to introduce the [KS°] mutation, or by addition of module 3 into the existing two-module [KS°] mutant described in Khosla et al., “Synthesis of Polyketides from diketides,” U.S. Pat. No.6,080,555 (incorporated herein by reference).

While illustrated using PKS modules derived from the erythromycin PKS, DEBS, other sources of modules may be used in accord with the methods of the invention, including but not limited to the PKSs involved in the biosythesis of erythromycin, megalomicin, picromycin, narbomycin, oleandomycin, lankamycin, FK506, FK520 (ascomycin), rapamycin, epothilone, tylosin, spiramycin, rosamicin, geldanamycin, pimaricin, FR008, candicidin, avermectin, and the like.

In another aspect of the invention, compound (3) of formula

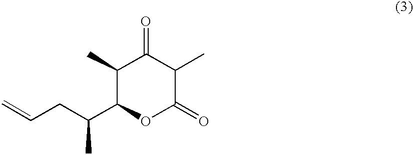

is prepared using an engineered polyketide synthase. This compound (3) is converted according to the methods of the invention through a short series of chemical transformations into (1), and thus serves as a precursor for the synthesis of polyketides such as epothilone. The stereochemistry of the C2-methyl group in (3) is unspecified, due to the ease of epimerization of this center and the utility of both diastereomers in the synthesis of epothilones.

The production of (3) according to the methods of the invention differs from the above-described production of (1) in that a methyltransferase domain is not used to add the second C2-methyl group in (1). A PKS comprising one module having a methylmalonyl-specific AT domain and (S)-specific KR domain and a second module having a methylmalonyl-specific AT domain and a thioesterase is provided by the invention for the conversion of Compound (2) into Compound (3).

One embodiment of the invention provides a two-module fragment of a naturally-occurring PKS comprising all the above-listed activities required for conversion of Compound (2) into Compound (3). An example of such a fragment is modules 5 and 6 of the narbonolide PKS from either Streptomyces venezuelae or Streptomyces narbonensis, (nar)[module(5)]-[module(6)]-TE. Compound (3) is produced according to the method of the invention when a growing culture of an organism, for example Streptomyces coelicolor CH999, containing an expression system for (nar)[module(5)]-[module(6)]-TE, is supplied with (2). The resulting (3) is extracted from the culture medium according to methods known in the art. Other heterologous expression hosts, including but not limited to Streptomyces lividans, Myxococcus xanthus, Escherichia coli, and Saccharomyces cerevesiae, may be used.

Another embodiment of the invention provides a two-module PKS for the conversion of (2) into (3) resulting from the genetic engineering of a two-module fragment taken from a naturally-occurring PKS. As an example, an engineered form of the DEBS3 protein of the erythromycin PKS is used, (ery)[module(5)]-[module(6)-KR°]-TE or (ery)[module(5)]-[module(6)-ΔKR]-TE. Two-module PKSs derived from natural PKSs other than DEBS are useful to convert (2) into (3). Modules 5 and 6 from the megalomicin PKS may also be used, for example.

In another embodiment of the invention, a PKS for the conversion of (2) into (3) resulting from the combination of two or more modules taken from different naturally-occurring PKSs is provided. Thus, a module from DEBS and a module from the narbonolide PKS may be combined, as in (ery)[module(5)]-(nar)[module(6)]-TE.

In yet another embodiment of the invention, a PKS containing more than two modules is provided for the conversion of (2) into (3), in which the first module serves as a loading module for (2) and is incapable of adding an extender unit onto the polyketide chain. An example is a three-module PKS comprised of modules 1, 2, and 3 of DEBS, in which the KS of module 1 has been inactivated through, for example, mutagenesis, along with the terminal thioesterase domain so as to provide (ery)[module(l)-KS°]-[module(2)]-[module(3)]-TE. Modules from several PKS genes can also be used, for example (ery)[module(l)-KS°]-[module(2)]-(nar)[module(6)]-TE.

While illustrated using PKS modules derived from the erythromycin PKS, DEBS, other sources of modules may be used in accord with the methods of the invention, including but not limited to the PKSs involved in the biosythesis of erythromycin, megalomicin, picromycin, narbomycin, oleandomycin, lankamycin, FK506, FK520 (ascomycin), rapamycin, epothilone, tylosin, spiramycin, rosamicin, geldanamycin, pimaricin, FR008, candicidin, avermectin, and the like.

Compound (3) is converted into (1) according to the methods of the invention by chemical methylation. Treatment of (3) with a base, for example sodium hydride, potassium tert-butoxide, and the like, followed by treatment with a methylation reagent, for example methyl iodide, converts (3) into (1).

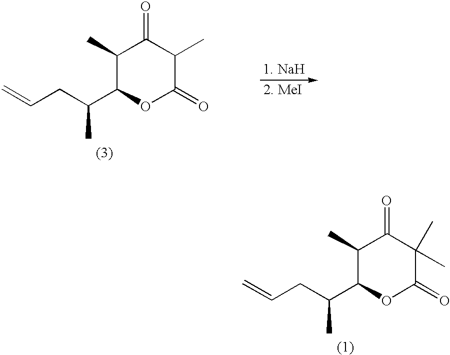

An illustrative protocol for a chemical methylation reaction is found in Example 7.

In another aspect of the invention, a compound of formula (4) is provided:

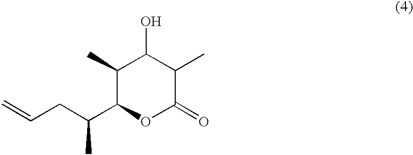

differing from (3) in having a C3-alcohol rather than a C3-ketone. According to the methods of the invention, (4) is converted into (3) by oxidation. The stereochemistry of the C3-alcohol is not specified, as both isomers yield (3) upon oxidation and so are useful according to the methods of the invention. The stereochemistry of the C2-methyl is not specified, as both isomers yield (1) upon oxidation and subsequent methylation, and so are useful according to the methods of the invention.

The production of (4) from (2) according to the methods of the invention differs from the above-described production of (3) in that a functional KR domain in the second module provides the C3-alcohol. A PKS comprising one module having a methylmalonyl-specific AT domain and (S)-specific KR domain and a second module having a methylmalonyl-specific AT domain, an active KR, and a thioesterase is provided by the invention for the conversion of (2) into (4).

One embodiment of the invention provides a two-module fragment of a naturally-occurring PKS comprising all the above-listed activities required for conversion of (2) into (4). As an example, the DEBS3 protein of the erythromycin PKS is used, (ery)[module(5)]-[module(6)]-TE. In this embodiment, DEBS3 is heterologously expressed in a host such as Streptomyces coelicolor CH999, Streptomyces lividans, Escherichia coli, or Saccharomyces cerevesiae. Two-module PKSs derived from natural PKSs other than DEBS are useful to convert (2) into (4), including but not limited to the PKSs involved in the biosythesis of erythromycin, megalomicin, picromycin, narbomycin, oleandomycin, lankamycin, FK506, FK520 (ascomycin), rapamycin, epothilone, tylosin, spiramycin, rosamicin, geldanamycin, pimaricin, FR008, candicidin, avermectin, and the like.

In another embodiment of the invention, a PKS for the conversion of (2) into (4) resulting from the combination of two or more modules taken from different naturally-occurring PKSs is provided. Thus, a module from DEBS and a module from the narbonolide PKS may be combined, for example, as in (nar)[module(5)]-(ery)[module(6)]-TE.

In yet another embodiment of the invention, a PKS containing more than two modules is provided for the conversion of (2) into (4), in which the first module serves as a loading module for (2) and is incapable of adding an extender unit onto the polyketide chain. An example is a three-module PKS comprised of modules 1, 5, and 6 of DEBS, in which the KS of module 1 has been inactivated through, for example, mutagenesis, along with the terminal thioesterase domain so as to provide (ery)[module(l)-KS°]-[module(5)]-[module(6)]-TE.

While illustrated using PKS modules derived from the erythromycin PKS, DEBS, other sources of modules may be used in accord with the methods of the invention, including but not limited to the PKSs involved in the biosythesis of erythromycin, megalomicin, picromycin, narbomycin, oleandomycin, lankamycin, FK506, FK520 (ascomycin), rapamycin, epothilone, tylosin, spiramycin, rosamicin, geldanamycin, pimaricin, FR008, candicidin, avermectin, and the like.

Although additional chemical modifications are necessary with some of these embodiments, the methods of the invention nevertheless provide the following advantages. First, the methods are relatively inexpensive. Due to the inherent stereoselectivity of biological systems, a racemic mixture of the SNAc compound may be used instead of as a pure enantiomer. Second, the expression of DEBS and other PKS genes in Streptomyces coelicolor is well characterized and the hydroxy lactone product may be made in relatively large quantities by simple fermentations.

The hydroxyl group of (4), which will become the future C-5 keto group of epothilone, may be chemically oxidized according to the methods of the invention with a mild oxidizing agent such as methylsufoxide/oxalyl chloride/triethylamine (i.e., Swernoxidation), methylsulfoxide/carbodiimide (i.e., Moffat oxidation), chormic acid (H₂CrO₄), hypervalent iodine oxidants (e.g., IBX, Dess-martin periodinane), and the like to provide (3). An illustrative example of such an oxidative protocol is found in Example 6.

In a further aspect of the invention, methods for the conversion of (3) into Compound A are provided. Scheme 1 illustrates one embodiment of the invention.

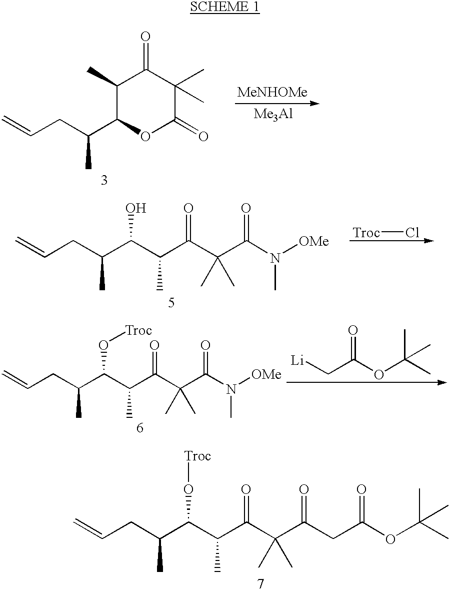

Briefly, the lactone of (3) is opened using N,O-dimethylhydroxylamine and trimethylaluminum to form the Weinreb amide, (5), and the resulting free hydroxyl group is protected using trichloroethyl chloroformate (“Troc-Cl”) to provide (6). The Weinreb amide is reacted with a source of nucleophilic acetate, for example the lithium enolate of tert-butyl acetate to yield (7), Compound A wherein P¹ is 2,2,2-trichloroethoxycarbonyl (“Troc”), p² is tert-butyl, and X is oxygen. Substitution of trichloroethyl chloroformate by another protecting reagent, including but not limited to trisubstituted silyl chlorides, trisubstituted silyl trifluoromethanesulfonates (“triflates”), and the like, gives rise to Compound A wherein p¹ is the corresponding protecting group, for example a trialkylsilyl ether. Particularly preferred examples include P¹=Troc, tert-butyldimethylsilyl (“TBS”), and triethylsilyl (“TES”).

In a second embodiment of the invention, the tert-butyl acetate of Scheme 1 is replaced with tert-butyl thioacetate, resulting in the formation of Compound A wherein p¹ is Troc, TBS, or TES, p² is tert-butyl, and X is sulfur.

In another aspect of the present invention, Compound B is provided that already includes the future C-3 alcohol with the appropriate stereochemistry:

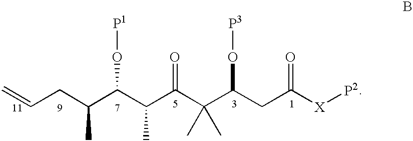

Wherein P¹, p², and p³ are H or protecting groups and X is oxygen or sulfur.

One embodiment for making Compound B from (6), and hence from (3), according to the methods of the present invention is illustrated by Scheme 2.

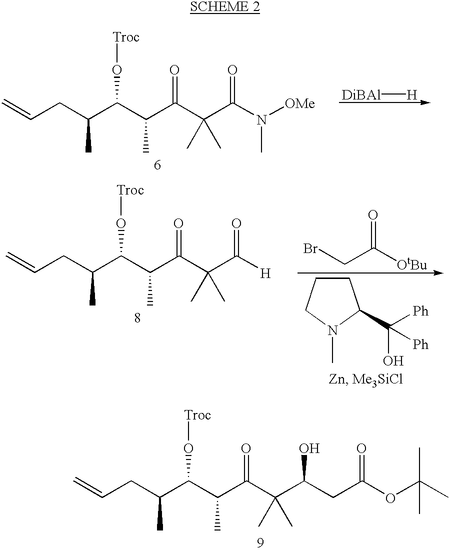

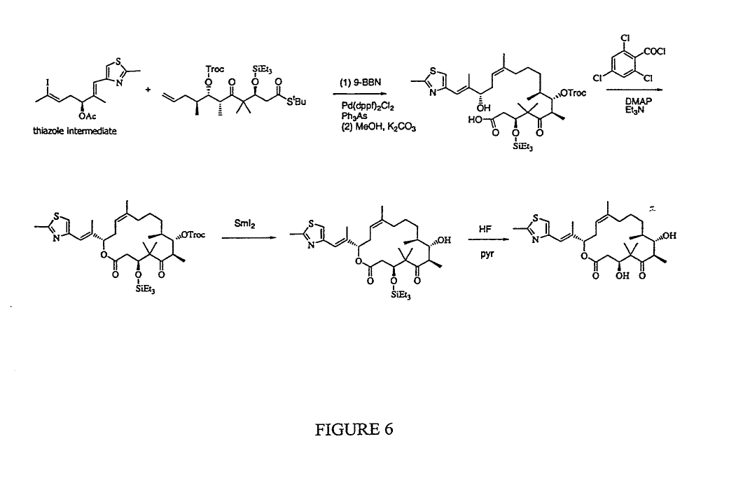

As described above, (1) is converted into (6). Di-isobutyl aluminum hydride is used to reduce the amide to aldehyde (8). An aldol or similar reaction subsequently stereoselectively extends the aldehyde by two carbons and sets the stereochemistry of the 3-alcohol. In one embodiment, an asymmetric Reformatsky reaction is performed using tert-butyl bromoacetate and zinc in the present of a chiral proline-derived ligand. In this embodiment, the product is (9), Compound B, wherein P¹=Troc, p²=tert-butyl, p³=H, and X=O. This is reacted with a hydroxyl protecting reagent, for example triethylchlorosilane, to protect the 3—OH to give (10), compound B wherein P¹=Troc, p²=tert-butyl, P³=Et₃Si (“TES”), and X=O. Use of reagents other than triethylchlorosilane can be used to introduce other p³ protecting groups. Preferred examples of P³ protecting groups include TES and TBS. According to the methods of the present invention (FIG. 6), (10) and the Danishefsky thiazole intermediate are coupled via the Suzuki coupling method, lactonized, and deprotected to yield epothilone D.

In another aspect of the present invention, Compound B, wherein P¹=H, p²=H, P³H, and X=O (11), is made directly using biological methods. The method generally involves the use of a library of polyketides where the library includes information regarding the structures of the polyketides. In preferred embodiments, the polyketide structures in the library are represented in linear form. The linear form may be a linearized version of the chemical structure. The linearized polyketide structures are a convenient representation for comparing one polyketide structure with another. In more preferred embodiments, each linearized polyketide structure is divided into a sequence of two carbon units. In even more preferred embodiments, the chemical structures of the two-carbon units are represented by symbols such that the structural sequence of two carbon units are now transformed into a linear sequence of symbols. The linearized structures are further decomposed into two-carbon units which in turn are each represented by a symbol. A general method for assigning symbols to structural fragments is described in greater detail in U.S. patent application 01/17352. Briefly, a modified version of the CHUCKLES methodology is provided to represent polyketide structures (see Siani et al., CHUCKLES: a method for representing and searching peptide and peptoid sequence, J. Chem. Inf. Comp. Sci, 1994 34:588-593 which is incorporated herein by reference).

The precise method for designing a gene for making a polyketide compound such as (11) depends on how the library of polyketides is organized. In general, the method comprises comparing the structure of the compound to be synthesized with the structures of the polyketides in the library and using this information to design a gene that when expressed in cells will make a PKS enzyme that can be used to synthesize the desired compound.

In one embodiment, (11), having the formula

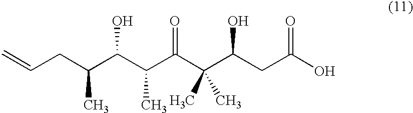

is made by using a subset of the epothilone PKS gene since epothilone itself includes the most number of consecutive matching two-carbon units with the target sequence (Compound 11) of two-carbon units. As a result, a variation of the inventive method is used. The general method comprises:

describing at least a portion of the target compound as a sequence of two-carbon units;

associating a PKS gene that results in the production of the naturally occurring polyketide;

determining the fragment of a PKS gene that is associated with making the compound's sequence of two-carbon units and;

designing a new gene that includes the fragment of the PKS gene.

The epothilone PKS gene has been cloned as described by PCT publication No. WO/0031247, which is incorporated herein by reference. As it can be seen from comparing the structure of (11) to a linearized representation of the epothilone structure

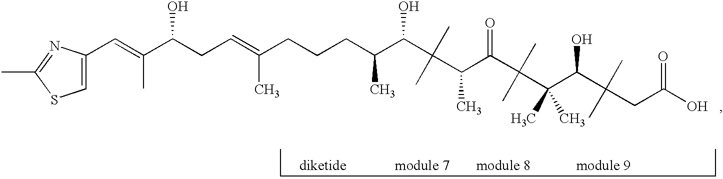

Compound (11) (not surprisingly) is virtually identical to the right hand portion of the epothilone structure. In addition, the right hand portion may be divided into four two-carbon units. Thus in one method, (2) is added to a PKS system comprising a functional EpoE and EpoF subunits, (epo)[module(7)]-[module(8)]-[module(9)]-TE. The functional PKS system may be in a suitable host cell or may be part of a cell free system.

A number of alternate methods of the present invention can be used to make (11) (or any other polyketide. When (11) is made using non-epothilone PKS genes, practice of the present invention is as described previously. For example, in another embodiment, a subset of modules from tartralone B (“tar”) is used to make (11) A linearized representation of tartralone B is as follows:

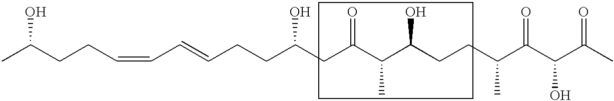

where the boxed portion of the structure is due to extender modules 5 through 7 of the tartralone B PKS enzyme. As a result, a compound (12) similar to (11) having the formula

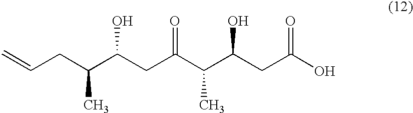

may be made by adding (2) to a PKS system comprising (tar)[module(5)]-[module(6)]-[module(7)]-TE. Compound (11) can be made by modifing the PKS system by replacing the AT in tartralone B extender module 5 to an AT specific for methyl malonyl CoA (e.g. the AT in tartralone B extender module 6) and inserting the methyl transferase domain from epothilone extender module 8 into tartralone B extender module 6, thus producing (tar) [module(5)-AT/tarAT6]-[module(6)+epoMT8]-[module(7)]-TE.

In yet another embodiment, a similar method is used to make (11) except that borophycin (“bor”) extender modules 4 through 6 are used instead of the tartralone B extender modules. If Compound (2) is added to a host cell expressing (bor)[module(4)]-[module(5)]-[module(6)]-TE, then the following polyketide (13) would result:

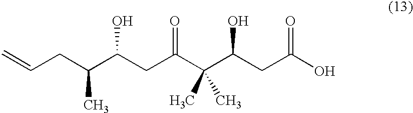

Compound (11) is made by modifying the AT in the borophycin extender module 4 to one specific for methyl malonyl CoA, for example in (bor)[module(4)-AT/tarAT6]-[module(5)]-[module(6)]-TE.

Similarly, modified portions of the aplasmomycin (“apl”) and boromycin PKS genes may be used to make (11). Illustrative examples of modified gene constructs are:

(apl)[module(3)-AT/tarAT6-DH°]-[module(4)]-[module(5)]-TE

(bor)[module(3)-AT/rapAT2-DH°]-[module(4)]-[module(5)]-TE

While the boromycin module 3 contains the complete set of DH/ER/KR modification domains, inactivation of the DH domain is sufficient to insure inactivation of the ER domain, as DH activity is a prerequisite for ER activity.

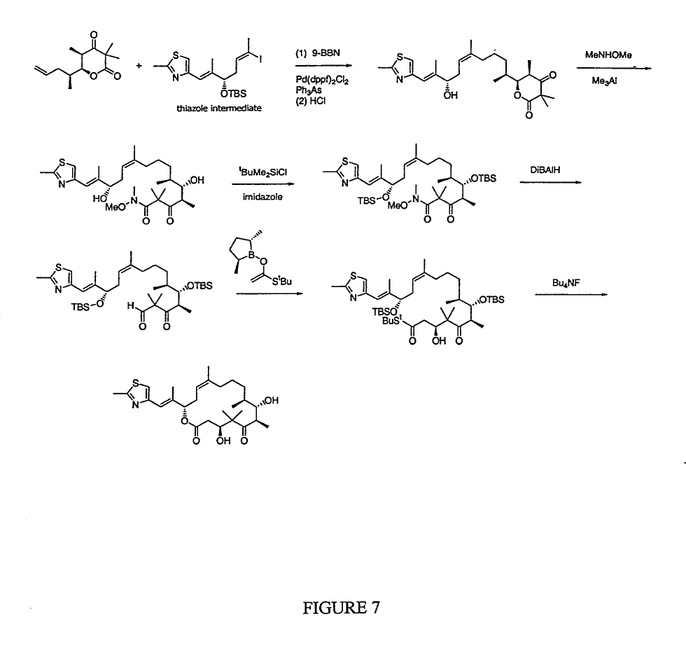

In another embodiment, the previously described (1) is used in another novel protocol to yield epothilone D. As illustrated by FIG. 7, the methylated keto-lactone is coupled to the thiazole intermediate using Suzuki coupling method. The extended lactone is opened to the Weinreb amide and the resulting free hydroxyl is protected. The amide is reduced to an aldehyde and is extended by two carbons. The resulting product is reacted with Bu₄NF which removes the protecting groups and forms the lactone at the same time to yield epothilone D.

Discodermolide

In another aspect of the present invention, a combination of biological and chemical methods is provided for making discodermolide analogs and intermediates useful in the synthesis of discodermolide analogs. Initial studies of discodermolide in tubulin polymerization assays suggest it possesses potent anti-cancer properties. As with epothilone, more extensive investigations are hampered by the small quantities of discodermolide that can be obtained from naturally occurring sources.

In an effort to circumvent this problem, several research groups have succeeded in completing the de novo chemical syntheses of discodermolide. Of these, the synthesis reported by Smith and co-workers (Scheme 3) is particularly notable because it uses a modular approach where three fragments that are derived from a common precursor, (14), are joined together. See e.g., Smith et al., 1999, Gram-scale synthesis of (+)-discodermolide, Org Lett. 1(11): 1823-6; U.S. Pat. Nos. 6,096,904, 6,031,133, and, 5,789,605; and PCT Publication Nos. WO 00/04865 and WO 98/24429, each of which is incorporated herein by reference.

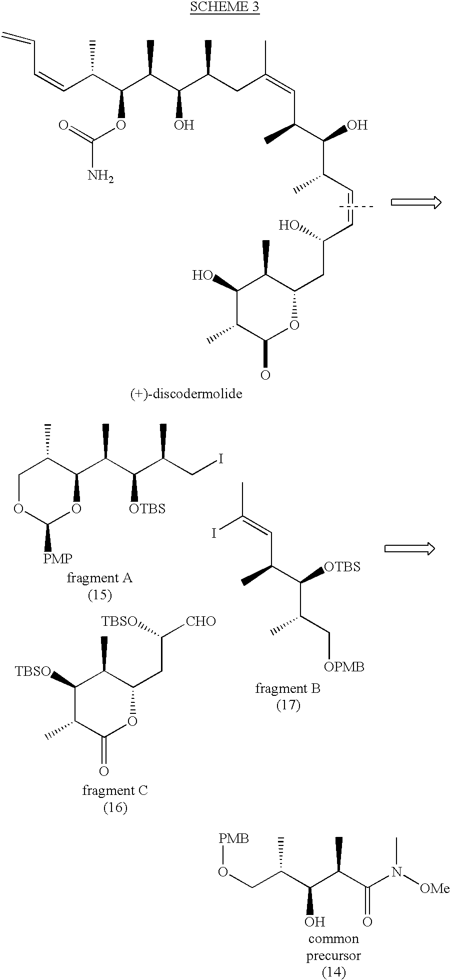

The Smith synthesis is long and complex, and generally not amenable for making commercial quantities. Because discodermolide is not the only polyketide whose use is hampered by inadequate supply, a need exists for novel approaches for obtaining these important compounds.

In one embodiment of the present invention, Smith's common precursor (14) is made using a combination of biological and chemical methods. Smith's common precursor is a compound of formula (C)

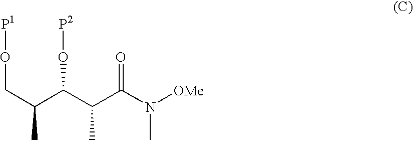

wherein p¹ is 4-methoxybenzyl (“PMB”) and p² is H.

The method of the invention comprises adding a compound of formula (D):

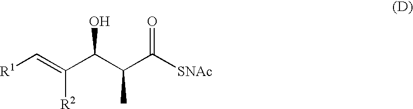

wherein

R¹ is H, alkyl, or aryl; and

R² is H or alkyl;

to a functional PKS system to make a lactone (E) of the following formula

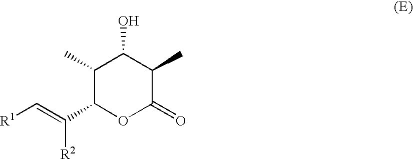

wherein

R¹ is H, alkyl, or aryl; and

R² is H or alkyl.

Preferred examples of suitable functional PKSs include (ery)[module(1 )-KS°]-[module(2)]-TE, (ole)[module(l)-KS°]-[module(2)]-TE, and (meg)[module(l)-KS°]-[module(2)]-TE. Scheme 4 illustrates one embodiment of the invention, wherein R² is H, where the lactone (E) resulting from feeding Compound (D) to the (ery)[module(1)-KS°]-[module(2)]-TE polyketide synthase is chemically modified to yield Smith's chiral precursor (14).

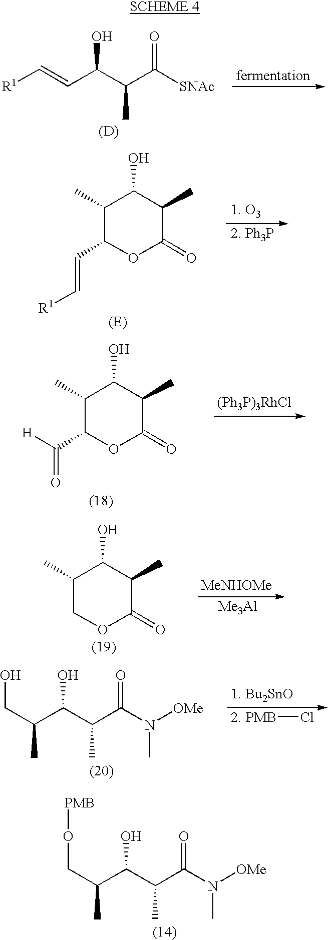

The vinyl group of the lactone is oxidized to the aldehyde (18), for example using ozonolysis with a reductive workup or using a two-step process of osmium tetraoxide followed by sodium periodate. The resulting aldehyde is decarbonylated to yield (19), for example using Wilkinson's catalyst (tris(triphenylphosphine)rhodium chloride). Lactone (19) can be converted into (14) by conversion to the Weinreb amide (20) followed by selective protection of the terminal primary alcohol. In a preferred example, this protection is performed by initial formation of the cyclic dibutylstannoxane, followed by reaction with an alkylating agent such as 4-methoxybenzylchloride.

In another embodiment of the invention, the lactone (22) resulting from feeding compound (D) wherein R¹ is H and R² is methyl (21) to a host cell expressing the (ery)[module(l)-KS°]-[module(2)-KR/rapKR2]-TE PKS or equivalent is used as shown in Scheme 5 to prepare Smith's “fragment A” of discodermolide (15).

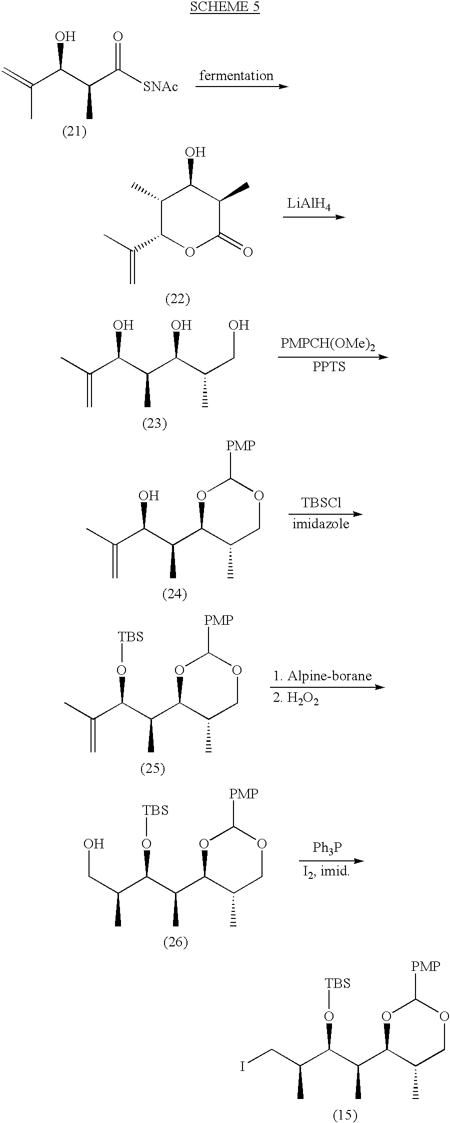

Replacement of the KR2 in DEBS1+TE with the module 2 KR from the rapamycin PKS results in formation of the lactone (22) having the opposite stereochemistry at the 3-alcohol. Lactone (22) is reduced, for example with lithium aluminum hydride, and the resulting triol (23) is protected by sequential treatment with (4-methoxyphenyl)benzaldehyde dimethyl acetal (PMPCH(OMe)₂) under acid catalysis, such as pyridinium para-toluenesulfonate (PPTS) followed by tert-butyldimethylsilyl chloride and base to yield (25). The alkene is stereoselectively hydroborated using Alpine-borane (B-isopinocampheyl-9-borabicyclo[3.3.1]nonane) with an oxidative workup to provide the primary alcohol (26). Conversion of the alcohol to the primary iodide using iodine and triphenylphosphine in the present of imidazole provides (15).

In another embodiment, genetically-engineered polyketide synthases are provided which 15 yield compounds useful in the preparation of the C15-C24 segment of discodermolide more directly:

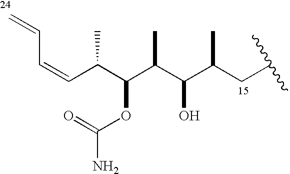

Comparison with the saw-tooth representation of 6-dEB reveals that this stereochemically complex segment of discodermolide may be produced by the protein that produces the C1-C7 fragment of 6-dEB:

[chemical structure]

In one embodiment of the invention, a PKS is constructed and used to produce a compound (27) of the formula

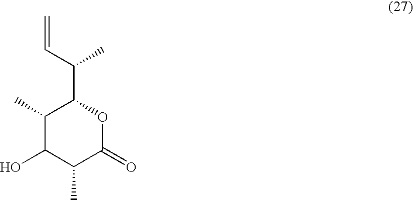

which serves as a precursor for synthesis of the C15-C24 segment of discodermolide. Examination of the sawtooth projection of (27) reveals that a suitable PKS would contain two modules equivalent in function to DEBS modules 5 and 6, found in the DEBS3 protein, along with the TE domain. Thus, in this embodiment, heterologously expressed (ery)[module(5)]-[module(6)]-TE is supplied with a thioester of the formula (28)

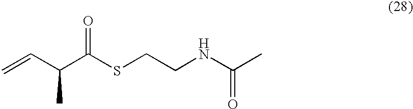

in order to produce (27). Again, while illustrated using modules taken from the DEBS gene cluster, other modules containing equivalent domains are suitable for the method of the invention.

In a second embodiment, a three-module construct (ery)[module(1)-KS°]-[module(5)]-[module(6)]-TE is constructed. Supplying a host cell expressing this PKS with (28) also results in the formation of (27).

In another aspect of the invention, 6-dEB analogs of formula (F) useful in the synthesis of discodermolide and other polyketides are provided:

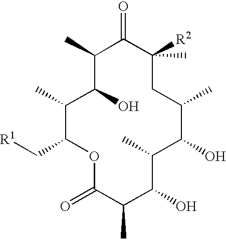

wherein R¹ is halogen or phenylthio; and R² is H or OH. The compounds are prepared by first contacting a PKS, for example the complete erythromycin PKS containing the KS1° mutation, with a thioester of formula (G)

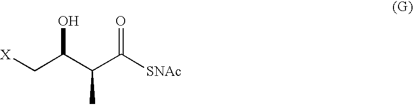

so as to produce compounds of formula (F) wherein R²=H, then optionally contacting said product with a cytochrome P450-type hydroxylase specific for hydroxylation at C8 to produce compounds of formula (F) wherein R²=OH.

Such hydroxylases are known from natural gene clusters, for example the oleandomycin and lankamycin gene clusters from Streptomyces antibioticus and Streptomyces violaceoniger, respectively. In oleandomycin, the oleP gene encodes a cytochrome P450 hydroxylase which adds a hydroxy group to the 8-position of 6-dEB, as described in McDaniel et al., “Production of 8,81-dihydroxy-6-deoxyerythronolide B,” U.S. patent application 09/768,927 (incorporated herein by reference). In lankamycin, the lkm P450 gene encodes an 8-hydroxylase.

The use of racemic precursors (G) to produce 6-dEB analogs (F) wherein R²=H is described in Ashley et al., “Synthesis of oligoketides,” PCT application no. US00/02397 (incorporated herein by reference).

In one embodiment of the invention, analogs (F) wherein R²=H are converted into intermediates useful in the synthesis of discodermolide, illustrated in Scheme 6.

[chemical structure]

Bayer-Villager oxidation of the C9-ketone of (F) using a peracid, for example trifluoroperacetic acid, peracetic acid, m-chloroperbenzoic acid, monopermaleic acid, and the like, yields the ring-expanded dilactone (G). When R¹=SPh, the peracid treatment also oxidizes the thioether into the sulfone SO₂Ph. In one embodiment of the invention, (G) is treated with zinc followed by methanolic base, generated for example by the use of potassium carbonate in methanol, to cleave the molecule into two fragments, acid (29) and lactone (30). In a second embodiment of the invention, the dilactone is treated with methanolic base directly to generate fragments (30) and (31).

Lactone (30) contains the stereogenic centers present in the Cl 5-C24 fragment of discodermolide, and in an embodiment of the invention (Scheme 7) is converted into a fragment suitable for the synthesis of discodermolide and analogs.

[chemical structure]

Elimination of the C8-alcohol, for example by activation through conversion to a sulfonate ester (tosylate, mesylate, triflate, and the like) followed by treatment with a base (for example, 1,8-diazabicyclo[5.4.0]undec-7-ene, “DBU”) yields the 7,8-alkene (32). The 3-hydroxyl group is protected, for example using a trialkylsilyl ether (33). Preferred protecting groups include triisopropylsilyl (TIPS), TES, and TBS. Ozonolysis of the alkene to the aldehyde is followed by installation of the discodermolide diene segment to yield intermediate (34). Several methods for construction of the diene (35) from the aldehyde are known, including the use of trimethylsilylallylboronates, Harried et al, “Total synthesis of (−)discodermolide: An application of a chelation-controlled alkylation reaction,” J. Org. Chem. (1997) 62:6098-6099; α-(trimethylsilyl)allyl bromide, Marshall et al., “Synthesis of discodermolide subunits by S_{E} 2′ addition of nonracemic allenylstannanes to aldehydes,” J. Org. Chem. (1998) 63:817-823; and phosphinoallyl titanium reagents, Smith et al., “Gram-scale synthesis of(+)-discodermolide,” Organic Letters (1999),1:1823-1826 (each of which is incorporated herein by reference). The preferred method for introducing the diene to form (XV) is the addition of a 3-(trialkylsilyl)allylboronate, particularly dimethyl 3-(trimethylsilyl)allylboronate, to the aldehyde, followed by treatment with a strong base, particularly KH.

Elaboration of intermediate (35) into discodermolide intermediate (38) according to the present invention involves reduction of the lactone carbonyl to a primary alcohol followed by differential protection of the resulting hydroxyl groups and final conversion of the primary alcohol into a group suitable for coupling to a second discodermolide fragment.

In one embodiment, (35) is first reduced using a hydride reagent. Preferred examples of such hydride reagents include lithium aluminum hydride, lithium triethylborohydride, and sodium borohydride. The resulting primary alcohol is selective protected. Preferred examples of protecting groups include the isobutyrate ester, acetate ester, benzoate ester, triphenyhnethyl ether, and di(4-methoxyphenyl)phenylmethyl ether. The isobutyrate ester is particularly preferred. The remaining secondary alcohol is then protected as a p-methoxybenzyl (PMB) ether, introduced either by reaction with p-methoxybenzyl trichloroacetimidate under acid catalysis or by reaction with p-methoxybenzyl bromide under base catalysis. Preferred examples of acid catalysts include trifluoromethanesulfonic acid and pyridinium p-toluenesulfonate (PPTS). Preferred examples of base catalysts include pyridine, diisopropylethylamine, and sodium hydride. After introduction of the PMB ether, the primary alcohol is deprotected to yield (37). When the protecting group is an ester, deprotection uses a mixture of potassium carbonate in methanol. When the protecting group is a trityl or DMT ether, deprotection uses chlorocatechol borane in methanol.

According to the methods of the invention, the liberated primary alcohol is converted into the iodide using triphenylphosphine and iodine in the presence of a base such as imidazole, yielding discodermolide fragment (38). This fragment can be incorporated into discodermolide or discodermolide analogs using methods known in the art, for example Marshall and Johns, “Total synthesis of (+)-discodermolide,” J. Org. Chem. (1998), 63:7885-7892, incorporated herein by reference.

In another embodiment of the invention, fragment (29) is used to prepare the Smith “chiral precursor” (14). As shown in Scheme 8, the alkene of (XI) is oxidized to an aldehyde. Preferred methods for oxidation include ozonolysis and osmium tetraoxide/sodium periodate; ozonolysis is particularly preferred. Reduction of the aldehyde to the alcohol (39), preferably by treatment of the intermediate ozonide with sodium borohydride, followed by acid-catalyzed lactonization provides compound (19), which is converted into the Smith precursor as described above.

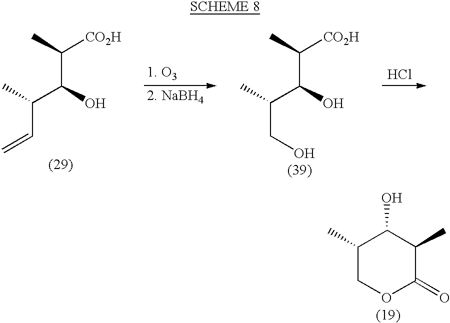

In another embodiment, (29) is used to prepare another intermediate useful in the synthesis of discodermolide and its analogs. As shown in Scheme 9, the alkene of (29) is oxidized to an aldehyde. Preferred methods for oxidation include ozonolysis and osmium tetraoxide/sodium periodate; ozonolysis is particularly preferred. The aldehyde is trapped as a methyl lactone acetal (40) by treatment with acidic methanol. The free hydroxyl group is protected. Preferred examples of protecting groups include trialkylsilanes such as TMS, TES, TBS, and TIPS; TIPS is particularly preferred. The lactone is opened to the Weinreb amide (41) by treatment with N,O-dimethylhydroxylamine. Compound (41) is used identically with compound “(−)-8” in Smith et al., “Gram-scale synthesis of (+)-discodermolide,” Organic Letters (1999)1:1823-1826, to produce discodermolide, differing only in the substitution of a TIPS protecting group for a TBS protecting group.

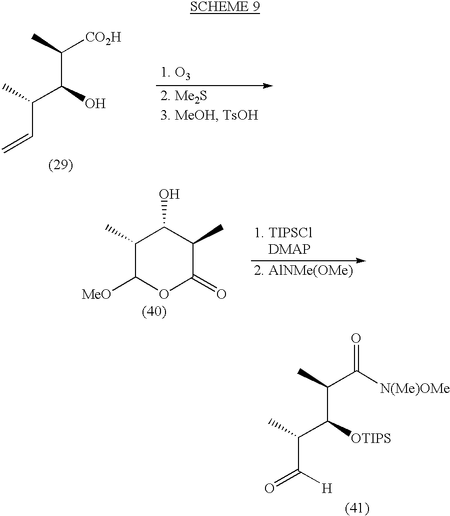

In another embodiment, (41) is used according to the methods of the invention as a precursor to the C9-C14 segment of discodermolide (Scheme 10). Reduction of the aldehyde using sodium borohydride is followed by protection of the alcohol with TBS to yield. Reduction of the Weinreb amide using diisobutylaluminum hydride (DiBAl-H) yields an aldehyde (42) which is converted into the vinyl iodide (43) using a Wittig reagent. Preferred examples of Wittig reagents include (iodomethylidene)triphenylphosphorane, (1-iodoethylidene)-triphenylphosphorane, and the like.

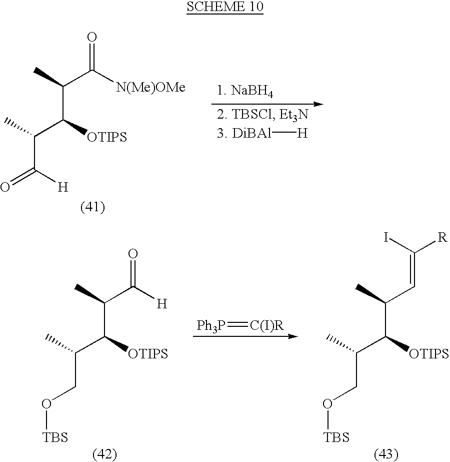

In another embodiment of the invention, the fragment (31) is used to prepare the C1-C8 segment of discodermolide. As shown in Scheme 11, fragment (31) is lactonized by treatment with an acid catalyst. Preferred examples of acid catalysts include 10-camphorsulfonic acid (“CSA”), toluenesulfonic acid, methanesulfonic acid, and the like. The resulting lactone (44) is differentially protected at the 6-alcohol, preferably using an ester, most preferably using an isobutyrate ester, and at the 3-alcohol, preferably using a trialkylsilyl ether, and most preferably using a TBS ether. The 6-alcohol is then deprotected using potassium carbonate in methanol, and then oxidized to an aldehyde (45). The oxidation is preferably performed using methylsulfoxide/oxalyl chloride/triethylamine (i.e., Swern oxidation), a hypervalent iodine reagent (Dess-martin periodinane or IBX), or chromium trioxide in pyridine, most preferably using methylsulfoxide/oxalyl chloride/triethylamine.

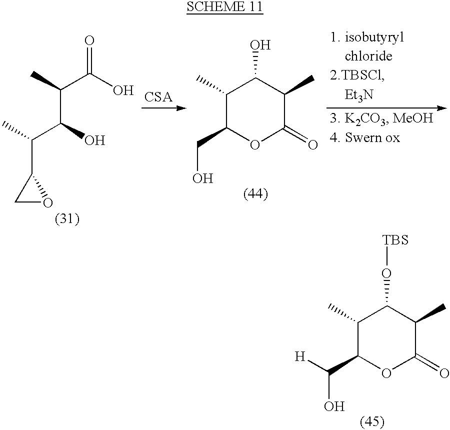

In one embodiment of the invention (Scheme 12), (45) is reacted with (ethylidene)triphenylphosphorane, producing compound (46). This is treated with bis(cyclopentadienyl)zirconium chloride hydride (Cp₂ZrHCl) to isomerize the alkene to the terminal position. Asymmetric dihydroxylation of the alkene using the stoichiometric osymlation conditions of Corey using a chiral C2-symmetric diamine ligand [N,N-bis(mesitylmethyl)-(R,R′)-1,2-diphenyl-1,2-diaminoethane] yields diol (47), which is again differentially protected to allow for oxidation of the terminal alcohol to the aldehyde (48). Compound (48) is converted into discodermolide according to Smith et al, “Gram-scale synthesis of(+)-discodermolide,” Organic Letters (1999)1:1823-1826.

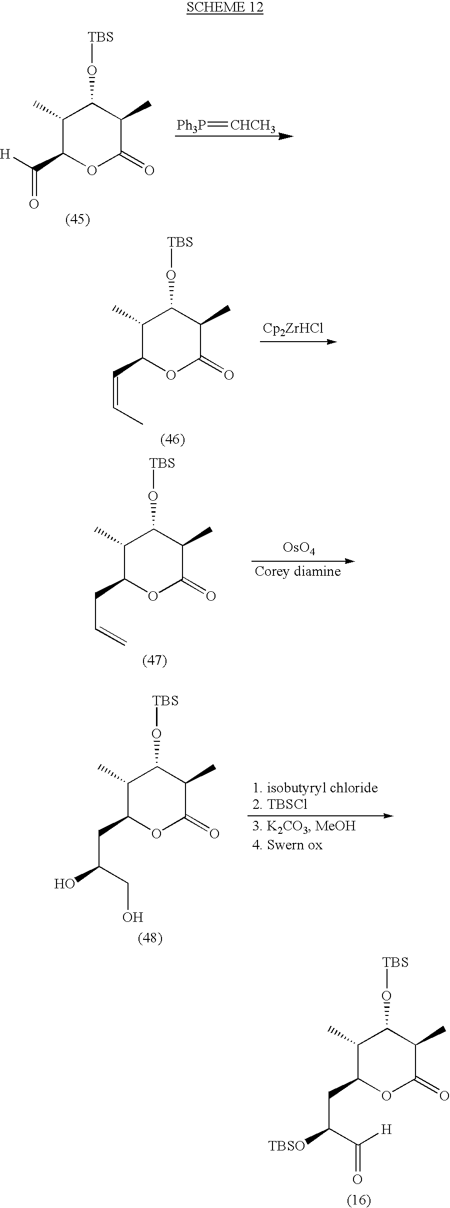

Completion of the synthesis of discodermolide according to the invention follows methods known in the art.

In another embodiment, compounds of formula (F)

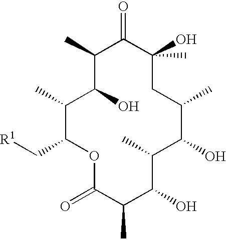

wherein R¹is H, halogen or phenylthio and R²=OH, are used to prepare intermediates (29), (30), and (31) described above, as illustrated in Scheme 13.

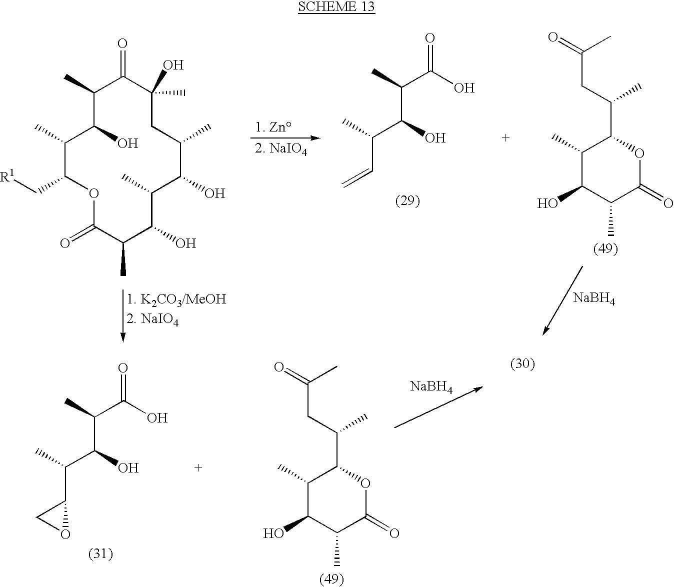

Treatment of (F) wherein R²=OH with a reagent capable of cleaving alpha-hydroxyketones, for example sodium periodate, results in fragmentation of the C8-C9 bond. When R¹=halogen or phenylthio, treatment with zinc as described above causes elimination of the halo lactone to give (29) and (49). When R¹=H, transesterification using basic methanol is used to cleave the lactone to give (31) and (49). Compound (49) is the ketone analog of (30), and so is converted into (30) by reaction with a reducing agent, such as sodium borohydride.

Discodermolide Compounds with C-14 Modifications

In another aspect of the invention, discodermolides having variations at C-14 are provided. As illustrated in Scheme 10 above, treatment of compound (42) with a Wittig reagent other than (1-iodoethylidene)-triphenylphosphorane yields discodermolides having groups other than methyl at C14. A preferred example of an alternate Wittig reagents is (iodomethylidene)-triphenylphosphorane, which results in the formation of 14-nordiscodermolide when used according to the Smith protocol.

In another embodiment, 14-nordiscodermolide compounds are made by coupling the appropriate fragments through a Wittig olefination rather than the palladium-mediated coupling of Smith et al. As shown in Scheme 14, the appropriate “A fragment” is prepared from intermediate (38) by homologation to form aldehyde (51).

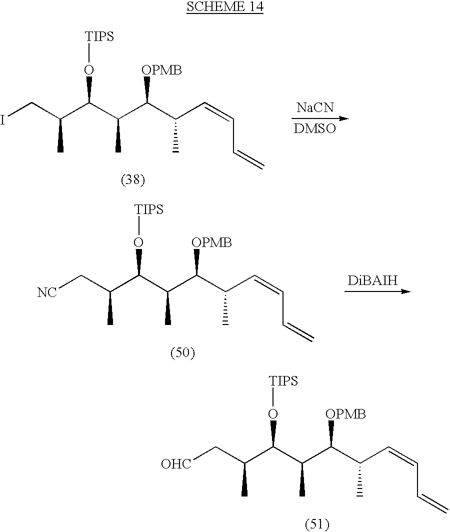

Aldehyde (51) is coupled with phosphorus ylid (52), derived from previously-described (42) as shown in Scheme 15 to yield the 14-nordiscodermolide intermediate (53).

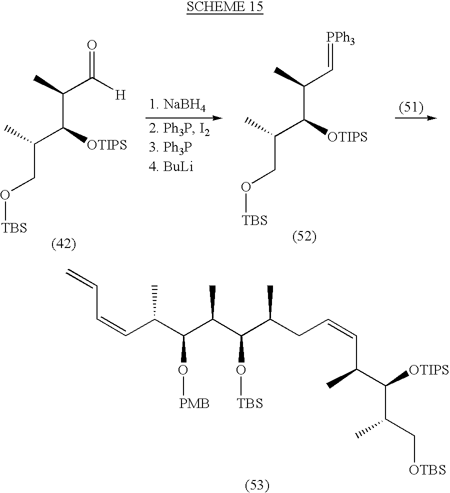

Subsequent conversion of (53) into 14-nordiscodermolide is illustrated in Scheme 16. Selective deprotection using mild acid treatment, for example brief exposure to HCl, yields alcohol (54) which is converted into the phosphonium salt (55) via the iodide. The ylid from 15 (55) is reacted with an aldehyde (R—CHO) comprising the “C fragment” or an analog in order to complete assembly of the discodermolide carbon skeleton.

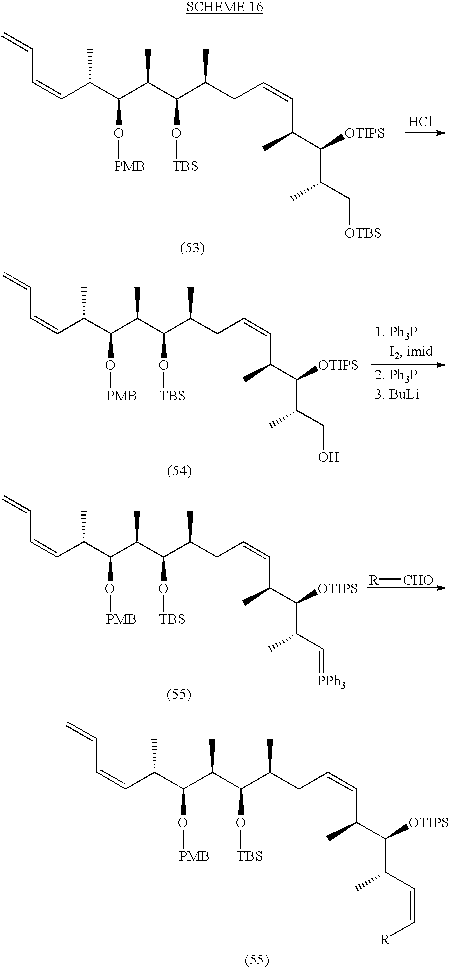

Discodermolide Compounds with “C-fragment” Modifications

The “C-fragment” of discodermolide, comprising C1-C7, is a particularly attractive target for analog production based on known structure-activity relationships. Hydroxyl groups at C-3 and C-7 positions may be converted into other groups such as acyl, alkoxy, aryloxy or other hydroxy protecting groups. Alternatively, the entire “C-fragment” may be replaced by other groups, for example lactams rather than lactones, or by simpler chemical analogs in which one or more of the functionalities present on the “C-fragment” are absent.

In one aspect of the invention, discodermolide compounds having hydrogen at C-3 and/or C-7 position are provided.

In another embodiment, (45) is reacted with (propylidene)triphenylphosphorane as shown in Scheme 17. The resulting alkene is treated with bis(cyclopentadienyl)zirconium hydride chloride to isomerize the alkene to the terminal position. Ozonolysis yields fragment (57), which is used to prepare 7-deoxydiscodermolide.

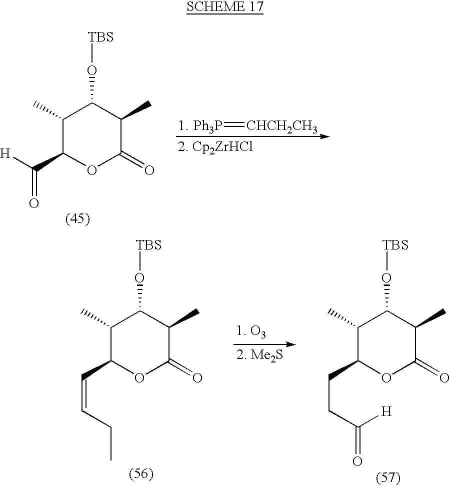

In a second embodiment, compound (56) is deprotected (Scheme 18) and the alcohol is removed by a Barton deoxygenation (thiocarbonyldiimidazole, followed by Bu₃SnH) to yield (58). Subsequent treatment as above yields fragment (59), which is useful in the synthesis of 3,7-dideoxydiscodermolides.

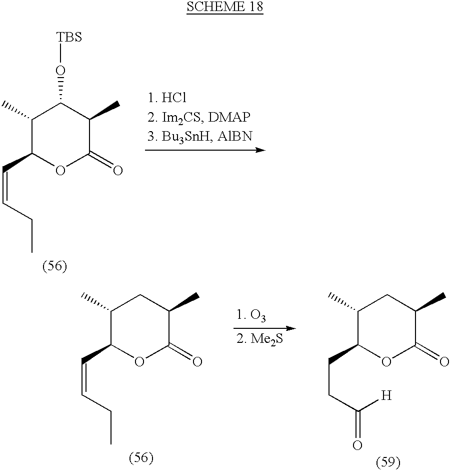

Lactam Discodermolide Compounds

In another aspect of the invention, fermentation-derived polyketides prepared using genetically-engineered PKSs are used to produce lactam analogs of discodermolides, i.e., 5-deoxy-5-aminodiscodermolides.

In one embodiment, lactam discodermolides are made using compound (64). One method for making (64) is outlined by Scheme 19.

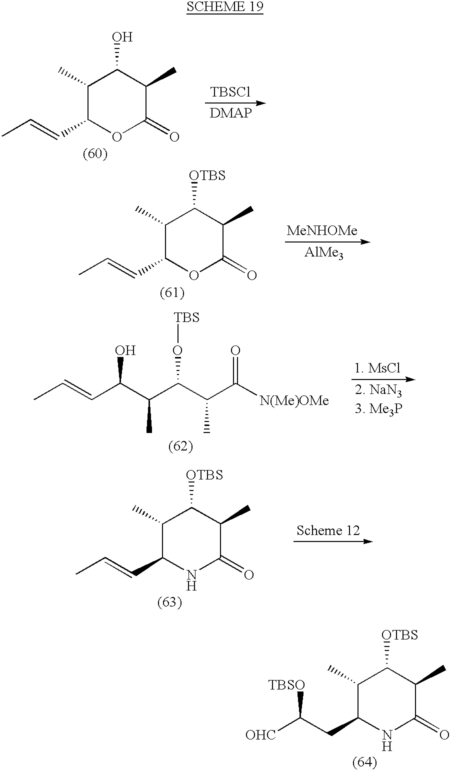

Lactone (E) wherein R¹=methyl and R²=H (60) is prepared as described above. Protection of the alcohol, preferably as the TBS silyl ether, followed by delactonization yields Weinreb amide (62). The allylic alcohol of (62) is activated, preferably as the mesylate, and displaced with inversion of configuration using azide. Staudinger reduction using trimethylphosphine gives lactam (63), which is converted into lactam fragment (64) according to the methods shown in Scheme 12 above.

In another embodiment of the invention, the nitrogen of lactam (63) is optionally alkylated by treatment with a stron base, preferably NaH, followed by reaction with a alkylating agent, for example methyl iodide (Scheme 20). In this manner, N-alkyl discodermolides are prepared.

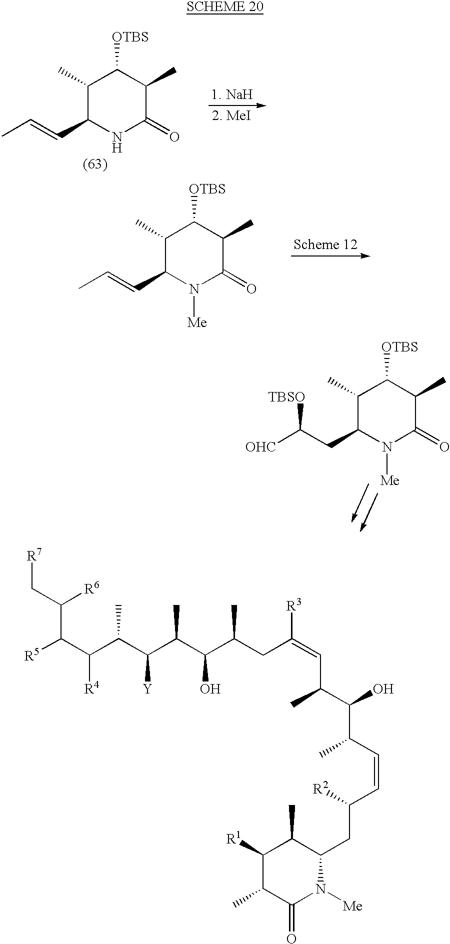

Any of the above-described compounds (57), (59), (64), and the like can replace the Smith “C-fragment” (16) in any of the previously described reactions to make discodermolide compounds, including 14-nordiscodermolides. Further, use of even simpler aldehydes in place of the “C-fragment” will give rise to further discodermolide analogs. Examples of such discodermolide analogs comprising simplified C-fragments are given in Hirokazu et al., “Compounds which mimic the chemical and biological properties of discodermolide,” PCT publication WO01/42179 (incorporated herein by reference).

A particularly preferred embodiment of the invention includes discodermolide analogs in which the “C-fragment” is replaced by a (3-hydroxyphenyl)ethyl group, such as compound (104).

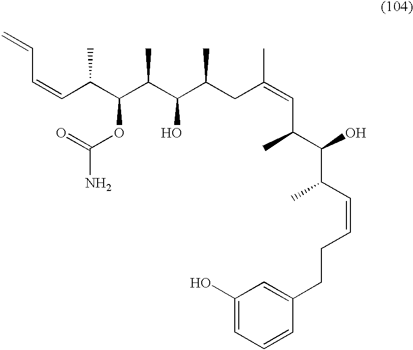

Compound (104) is prepared as described in Scheme 16 above, wherein “R—CHO” is 3-(3-tert-butyldimethylsilyloxy)phenylpropanal.

Discodermolide Compounds where Y is amino or -NHC(═O)NH₂.

In another aspect of the invention, discodermolide compounds where Y is amino or —NHC(═O)NH₂ are made using compound (71), a modified version of compound (37) in which the carbon center containing the PMB protected alcohol is the opposite stereochemistry. Compound (71) is prepared from fermentation-derived lactone (65), a compound of formula (E) wherein R¹=H and R²=methyl prepared using the (ery)[module(l)-KS°]-[module(2)]-TE PKS (Scheme 21).

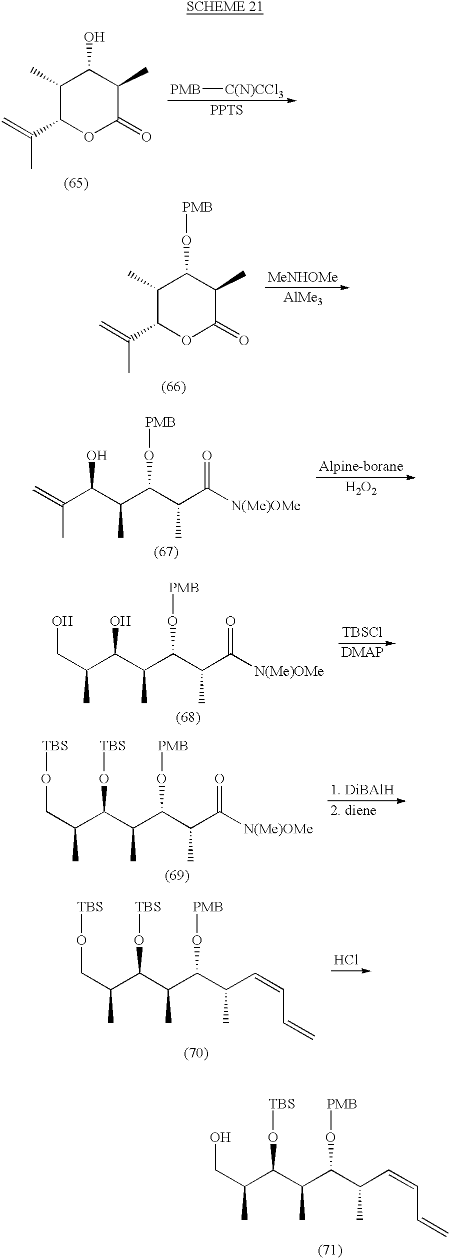

Lactone (65) is protected on the hydroxyl as the PMB ether, using PMB trichloroacetimidate and an acid catalyst, then is converted into Weinreb amide (67). Stereoselective hydroboration using Alpine-Borane followed by an oxidative workcup gives diol (68), which is protected as the bis-TBS ether (69). Reduction of the Weinreb amide to the aldehyde allows installation of the diene segment of discodermolide as described in Scheme 7 above. Selective deprotection of the primary alcohol provides (71).

Compound (71) may be combined with other compounds of the invention to prepare discodermolide derivatives having a hydroxyl at Y where the carbon containing Y (C19) is of the opposite stereochemistry that is normally found in discodermolide (100), including 14-nordiscodermolides and discodermolides having further modifications to the C-segment, such as deoxy analogs and lactams These compounds are converted into the 19-aminodiscodermolides by removal of the PMB ether (dichlorodicyanoquinone, DDQ) to give the alcohol (101), activation of the hydroxyl as the mesylate, displacement of the mesylate by azide with inversion of configuration at C19, and finally Staudinger reduction of the azide to the amine to give (102) (Scheme 22).

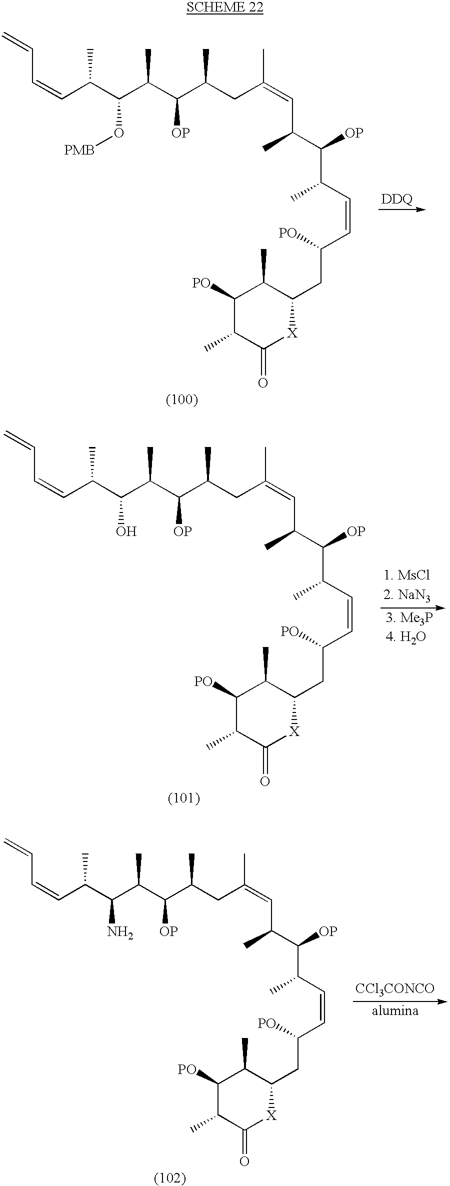

The amino group of compound (102) may be further modified using any number of standard reactions known in the art. In one preferred embodiment, the amino group of compound (102) is converted into an urea group to yield 19-urea compound (103) as shown in Scheme 21. In another preferred embodiment, the 19-urea analog (105) of compound (104) is prepared according to the methods of the invention.

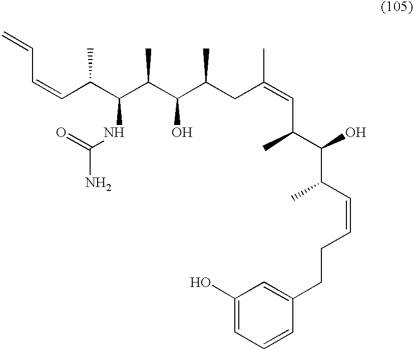

As illustrated by the above epothilone and discodermolide examples, there are a variety of methods to make a particular polyketide using chimeric PKS constructs of the invention. A number of alternate synthetic schemes are presented in U.S. patent application No. 60/224,038 filed Aug. 9, 2000 by inventors Daniel Santi and Gary Ashley entitled BIO-INTERMEDIATES FOR USE IN THE CHEMICAL SYNTHESIS OF POLYKETIDES (incorporated herein by reference). Numerous examples of these alternate procedures are given in the following Examples. Although the library of polyketide structures may be amended as additional polyketides become known, a sufficient diversity exists in the current library to make almost any polyketide structure using purely biological techniques. The number and diversity of the polyketides that may be made increases dramatically when the biological techniques described herein are combined with standard chemical strategies.

A detailed description of the invention having been provided above, the following examples are given for the purpose of illustrating the present invention and shall not be construed as being a limitation on the scope of the invention or claims.

### EXAMPLE 1

### 2-methyl-4-pentenoate N-acetysteamine thioester

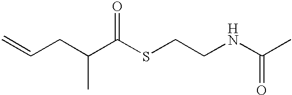

A solution of 2-methyl-4-pentenoic acid (1.14 g) and diphenyl phosphorylazide ( 3.0 g) in 10 mL of dry tetrahydrofuran cooled on ice was treated with 5 mL of dry triethylamine and allowed to stir for 30 minutes under inert atmosphere. Freshly distilled N-acetylcysteamine (1.5 g) was added and the mixture was allowed to stir for an additional hour. The reaction was quenched by addition of 10 mL of 2 N HCl, diluted with 50 mL of ethyl acetate, and the phases were separated. The organic phase was washed sequentially with water and brine, then dried over MgSO₄, filtered, and evaporated to yield the crude thioester as a yellow oil. Purification by silica gel chromatography (ether) afforded pure product as an oil.

¹H-NMR (CDCl₃, 400 MHz): δ 5.96 (1H, br s), 5.72 (1H, ddt, J=7.2,10.0,17.2 Hz), 5.07 (1H,dm, J=17.2 Hz), 5.04 (1H,dm,J=10.0 Hz), 3.42 (2H, m), 3.02 (2H,m), 2.74 (1H,sextet,J=7), 2.44 (1H,m), 2.18 (1H,m), 1.96 (3H,s), 1.90 (3H,d,J=7 Hz). ¹³C-NMR (CDCl₃, 100 MHz): δ 203.68, 170.49, 134.85, 117.25, 48.17, 39.76, 38.03, 28.20, 23.17, 17.13.

### EXAMPLE 2

### (±)-syn-2-methyl-3-hydroxy-4-hexenoate N-acetylcysteamine thioester

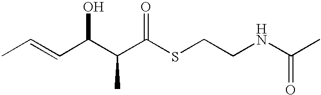

Step 1. A solution of N-propionyl-2-benzoxazolone (100.0 g) in anhydrous Ch₂CL₂ (1100 mL) was cooled to 3° C. with mechanical stirring under N₂ atmosphere. TiCl₄ (58.4 mL) was added at a rate such that the internal temperature remained below 10° C. (ca. 10 minutes). The resulting yellow slurry was stirred vigorously for 40 minutes, then triethylamine (87.4 mL) was added at a rate such that the internal temperature remained below 10° C. (ca. 10 minutes). The resulting deep red solution was stirred for 80 minutes. Crotonaldehyde (55 mL) was added at a rate such that the internal temperature remained below 10° C. (ca. 20 minutes), and the reaction was followed by thin-layer chromatography (4:1 hexaneslethyl acetate). After stirring for 90 minutes, the reaction was quenched by addition of 450 mL of 2 N HCl. The phases were separated, and the aqueous phase was extracted 3 times with 750-mL mL portions of ether. The organic phases were combined and washed three times with 200-mL mL portions of 2 N HCl. The acidic washes were combined and back-extracted 3 times with 150-mL portions of ether. The combined organic extract was washed once with 300 mL of sat. aq. NaHCO₃, and once with 300 mL of sat. aq. NaCl. The organic phase was then dried over MgSO₄, filtered, and concentrated under vacuum to a yellow slurry. The product was collected by vacuum filtration and rinsed with hexanes to yield a colorless solid. Concentration of the filtrate yielded a second crop of product, which was collected in the same manner, giving a combined 103 g (80% yield) of crystalline product; mp =123-4° C. The mother liquor can be chromatographed (4:1 hexanes/ethyl acetate) to yield additional product. mp 74-6° C. ¹H NMR (CDCl₃) δ8.06 (m, 1 H); 7.23 (m, 3 H); 5.78 (dqd, 1 H, J=15, 7, 1 Hz); 5.55 (ddq, 1 H, J=15, 7, 2 Hz); 4.52 (br, 1 H); 4.05 (qd, 1 H, J=7, 4 Hz); 2.38 (br d, 1 H, J=3 Hz); 1.70 (ddd, 3 H, J=7, 1, 1 Hz, 3 H); 1.30 (d, 3 H, J=7 Hz). ¹³C NMR (CDCl₃) δ 175.50, 151.20, 142.18, 129.99, 128.78, 127.80, 125.43, 124.86, 116.22, 109.85, 72.92, 44.31, 17.71, 11.04.

Step 2. One molar equivalent of sodium methoxide (25% w/v in methanol; ca. 150 mL) is added in a slow stream to a solution of N,S-diacetylcysteamine (173 g) in methanol (910 mL) under N₂. When half of the calculated volume has been added, the reaction is monitored by TLC (1:1 ethyl acetate/hexanes), and methoxide addition is continued until complete conversion of the N,S-diacetylcysteamine to N-acetylcysteamine. Acetic acid (50 g) is added, and the resulting solution of sodium thiolate is cannulated into a flask containing solid (±)-N-[syn-2-methyl-3-hydroxy-4-hexenoyl]-2-benzoxazolone (240 g) under N₂. After 15 minutes, the reaction is quenched with solid oxalic acid dihydrate (80.4 g), filtered, and concentrated to a yellow oil. The residue is dissolved in 2:1 hexanes/ethyl acetate and submitted to batch elution chromatography on SiO₂. The silica is washed with 2:1 hexanes/ethyl acetate to remove 2-benzoxazolone, then with ethyl acetate/methanol (9:1) to elute the product thioester. Evaporation of the thioester-containing eluent yields the product.

### EXAMPLE 3

### (±)-syn-4-methyl-3-hydroxy-4-hexenoate N-acetylcysteamine thioester

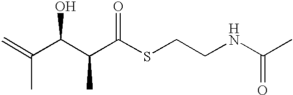

Step 1. A solution of N-propionyl-2-benzoxazolone (100.0 g) in anhydrous CH₂Cl₂ (1100 mL) is cooled to 3° C. with mechanical stirring under N₂ atmosphere. TiCl₄ (58.4 mL) is added at a rate such that the internal temperature remains below 10° C. (ca. 10 minutes). The resulting yellow slurry is stirred vigorously for 40 minutes, then triethylamine (87.4 mL) is added at a rate such that the internal temperature remains below 10° C. (ca. 10 minutes). The resulting deep red solution is stirred for 80 minutes. Methacrolein (55 mL) is added at a rate such that the internal temperature remains below 10° C. (ca. 20 minutes), and the reaction is followed by thin-layer chromatography (4:1 hexanes/ethyl acetate). After stirring for 90 minutes, the reaction is quenched by addition of 450 mL of 2 N HCl. The phases are separated, and the organic phase is filtered through a pad of silica gel. The silica gel is washed with ether, and the combined organic are concentrated under vacuum to a. The product is collected by vacuum filtration and rinsed with hexanes to yield a colorless solid.

Step 2. One molar equivalent of sodium methoxide (25% w/v in methanol; ca. 150 mL) is added in a slow stream to a solution of N,S-diacetylcysteamine (173 g) in methanol (910 mL) under N₂. When half of the calculated volume has been added, the reaction is monitored by TLC (1:1 ethyl acetate/hexanes), and methoxide addition is continued until complete conversion of the N,S-diacetylcysteamine to N-acetylcysteamine. Acetic acid (50 g) is added, and the resulting solution of sodium thiolate is cannulated into a flask containing solid (±)-N-[syn-4-methyl-3-hydroxy-4-hexenoyl]-2-benzoxazolone (240 g) under N₂. After 15 minutes, the reaction is quenched with solid oxalic acid dihydrate (80.4 g), filtered, and concentrated to a yellow oil. The residue is dissolved in 2:1 hexanes/ethyl acetate and submitted to batch elution chromatography on SiO₂. The silica is washed with 2:1 hexanes/ethyl acetate to remove 2-benzoxazolone, then with ethyl acetate/methanol (9:1) to elute the product thioester. Evaporation of the thioester-containing eluent yields the product.

### EXAMPLE 4

### General Procedure for Polyketide Production by Fermentation

Cultures are grown at 30° C. and 150-250 rpm in FKA basal medium (45 g/L starch, 10 g/L corn steep liquor, 10 g/L dried debittered brewer's yeast, 1 g/L calcium carbonate, and 23.8 g/L HEPES, free acid) (Sigma-Aldrich, St. Louis, Mo.). Shake flask medium pH is adjusted to pH 7.0 prior to sterilization by autoclaving for 90 min at 121° C. Bioreactor fermentation medium is prepared without HEPES buffer and autoclaved for 90 min at 121° C. After sterilization and cooling, the medium was adjusted to pH 6.5. All media are supplemented with 50 mg/L thiostrepton (Calbiochem, La Jolla, Calif.) in (50 mg/mL) DMSO and 10 mL/L of 50% (v/v) antifoam (Antifoam B, J. T.Baker, Phillipsburg, N.J.) as post-sterile additions. Strains are maintained as frozen cell banks prepared by adding glycerol (30% v/v final) to an exponentially growing culture (in FKA medium) and freezing 1 mL aliquots at −85° C. Thioester feedstocks (400 mg/mL) and thiostrepton (50 mg/mL) are prepared as DMSO solutions which were sterile filtered using 0.2 μm nylon membranes before addition to cultures.

Primary seed cultures are established by inoculating 50 mL of FKA with a cell bank vial and cultivating for 3 days. Glucose feeding is performed by the addition of a sterile filtered 50% (w/v) glucose solution as a single daily bolus to give the desired feed rate. Bioreactor fermentations are performed in B. Braun MD 5 L fermentors with 3 L of production medium operated at 30° C., 0.3 VVM airflow, and 600 rpm agitation. Dissolved oxygen concentration and pH are monitored using autoclaveable electrodes (Mettler Toledo, Wilmington, Mass.). Under these operating conditions, dissolved oxygen is maintained above 50% at all times. Foaming is controlled by automatic addition of 50% (v/v) Antifoam B solution. The pH is controlled by automatic addition of 2.5 N sodium hydroxide or sulfuric acid. Bioreactors are inoculated with 5% (v/v) secondary seed culture prepared by sub-culturing 25 mL of primary seed into 500 mL of FKA and cultivation for 2 days. Upon inoculation into the bioreactors, the thioester feedstock is added to a final concentration of 2 g/L, and the fermentation is allowed to proceed for 6 days. The cells are removed by centrifugation, and the broth is filtered through a column of XAD-16 to absorb polyketide products. After washing with 2 column volumes of water, the resin is eluted with acetone.

The eluate is evaporated to an aqueous slurry, which is extracted with ethyl acetate. The extract is dried and concentrated. The polyketide product is purified by silica gel chromatography.

### EXAMPLE 5

### (2R,3S,4S, 5S,6S)-3,5-dihydroxy-2.4,6-trimethyl-8-nonenoate δ-lactone

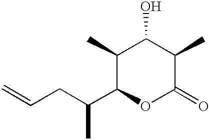

Method A

Streptomyces lividans K4-155 was transformed with plasmid pKOS10-153 containing the gene encoding the DEBS3 protein in a pSET type (an integrative) plasmid. An agar plug was used to inoculate a 5 mL seed culture of R⁶ broth and was grown for 2 days at 30° C. at 200 rpm. A 2.5 mL portion of this culture was used to inoculate 50 mL of R⁶ media in a 250 mL baffled flask and was incubated at 30° at 200 rpm. After 1 day, a solution of 2-methyl-4-pentenoate N-acetylcysteamine thioester was added (to a final concentration of 1 g/L from a 40% w/v solution in DMSO). Samples of broth were then taken from the flasks at intervals.

The broth from cultures with and without the addition of thioester were analyzed by LC-MS.

Method B

A solution of 2-methyl-4-pentenoate N-acetylcysteamine thioester (20 g/L) in methylsulfoxide is added to a 2-day old culture of Streptomyces coelicolor CH999 harboring a plasmid which contains the gene encoding the DEBS3 protein. The fermentation is performed as described until the general procedure of Example 4.

### EXAMPLE 6

### (2R,4R,5S,6S)-5-hydroxy-3-oxo-2,4,6-trimethyl-8-nonenoate δ-lactone

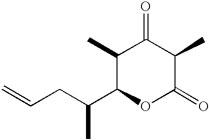

Dry methylsulfoxide (0.45 mL) is added dropwise to a solution of oxalyl chloride (0.28 mL) in 30 mL of CH₂Cl₂ under inert atmosphere at −78° C. After 10 minutes, a solution of (2R,3S,4S,5S,6S)-3,5-dihydroxy-2,4,6-trimethyl-8-nonenoate δ-lactone (600 mg) in 10 mL of CH₂Cl₂ is added dropwise, followed by triethylamine (2.0 mL). Stirring is continued for 20 minutes, then the reaction is allowed to warm to ambient temperature over 30 minutes. Brine (30 mL) is added and the mixture is extracted with CH₂Cl₂. The organic extracts are combined, washed with brine, dried over MgSO₄, filtered, and evaporated. The product is purified by silica chromatography.

### EXAMPLE 7

### (4R,5S,6S)-5-hydroxy-3-oxo-2,2,4,6-tetramethyl-8-nonenoate δ-lactone

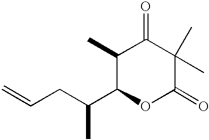

A solution of (2R,4R,5S,6S)-5-hydroxy-3-oxo-2,4,6-trimethyl-8-nonenoate δ-lactone (2.1 g) in 10 mL of tetrahydrofuran is added dropwise to a 0° C. suspension of sodium hydride (0.50 g of a 60% dispersion in oil) in 10 mL of tetrahydrofuran. Methyl iodide (2 g) is added, and the mixture is warmed to ambient temperature and stirred until complete as evidenced by thin layer chromatographic analysis. The reaction is diluted with ether, quenched by addition of 1 N HCl at 0° C., and the phases are separated. The organic phase is washed with brine, dried over MgSO₄, filtered, and evaporated. The crude product is purified by silica gel chromatography.

### EXAMPLE 8

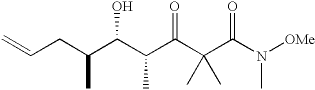

### N-Methyl-N-methoxy (4R,5S,6S)-5-hydroxy-3-oxo-2,4,6-tetramethyl-8-nonenoate amide

A solution of 2 M trimethylaluminum in toluene (10 mmol) is added dropwise to a suspension of N,O-dimethylhydroxylamine hydrochloride (10 mmol) in 8 mL of CH₂Cl₂ at 0° C. The resulting homogeneous solution is stirred for 30 min at ambient temperature. A solution of (4R,5S,6S)-5-hydroxy-3-oxo-2,2,4,6-tetramethyl-8-nonenoate □-lactone (2 mmol) in 4 mL of CH₂Cl₂ is added over 10 min, and the resulting solution is heated at reflux until complete consumption of starting material. Upon cooling, the mixture is poured into 1 M HCl, and extracted with CH₂Cl₂. The extract is washed sequentially with 5% NaHCO₃ and brine, dried over MgSO₄, filtered, and evaporated. The product is purified by silica gel chromatography.

### EXAMPLE 9

### N-Methyl-N-methoxy (4R, 5S, 6S)-5-((2,2,2-trichloroethoxy)carbonyloxy)-3-oxo-2,4,6 -tetramethyl-8-nonenoate amide

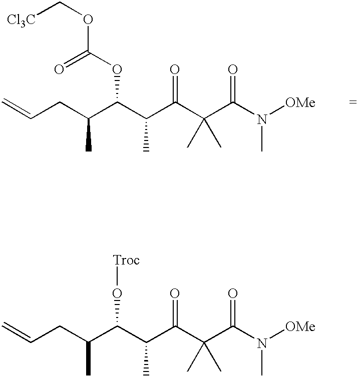

A solution of N-Methyl-N-methoxy (4R, 5S, 6S)-5-hydroxy-3-oxo-2,4,6-tetramethyl-8-nonenoate amide (1 mmol) and 2,2,2-trichloroethyl chloroformate (1.5 mmol) in pyridine (2 mL) is stirred overnight. The mixture is concetrated to dryness. The residue is redissolved in CH₂Cl₂ and washed sequentially with 1 N HCl, 5% NaHCO₃, and brine. After drying over MgSO₄, the solution is filtered and evaporated to dryness. The product is purified by silica gel chromatography.

### EXAMPLE 10

### (4R, 5S, 6)-5-((2,2,2-trichloroethoxy)carbonyloxy)-3-oxo-2,4,6-tetramethyl-8-nonenal

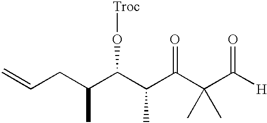

A solution of N-Methyl-N-methoxy (4R,5S,6S)-5-((2,2,2-trichloroethoxy)carbonyloxy)-3 -oxo-2,4,6-tetramethyl-8-nonenoate amide (10 mmol) in 20 mL of toluene is cooled to −78° C., and a 1.5 M solution of diisobutylaluminum hydride in toluene (50 mmol) is added dropwise over a 45 minute period. Stirring is continued until complete conversion of starting material as determined by thin-layer chromatographic analysis. Ethyl acetate (10 mL) is added, and the mixture is allowed to warm to ambient temperature. A 100-mL portion of 1 N HCl is added, and the mixture is stirred an additional 1 hour before extracting with CH₂Cl₂. The extract is dried over MgSO₄, filtered, and evaporated. The product is purified by silica gel chromatography.

### EXAMPLE 11

### Tert-butyl (3S,6R,7S,8S)-3-hydroxy-5-oxo-4,4,6 8-tetramethyl-7-(2,2,2 -trichloroethoxycarbonyl)-10-undecenoate

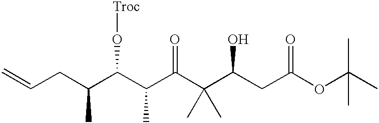

Tert-butyl acetate (1.0 mL) is added to a −78° C. solution of lithium diisopropylamide (8.7 mmol) in 35 mL of ether, and the mixture is tirred for 1 hour. A solution of bis[1,2:5,6-di-O-20 isopropylidene-α-L-glucofuranos-3-O-yl]cyclopentadienyltitanium chloride (9.7 mmol) in 100 mL of ether is added over 1 hour, and stirring is continued for an additional 30 minutes.

The reaction is allowed to warm to −30° C., kept 1 hour, then cooled to −78° C. A solution of (4R,5S,6S)-5-((2,2,2-trichloroethoxy)carbonyloxy)-3-oxo-2,4,6-tetramethyl-8-nonenal in 20 mL of ether is added and the reaction is stirred for 2 hours, quenched with aqueous THF, 25 then filtered through Celite and concentrated. The product is isolated by silica gel chromatography using 7% ethyl acetate in hexanes.

### EXAMPLE 12

### Tert-butyl (3S,6R,7S,8S)-3-(triethylsilyloxy)-5-oxo-4,4,6,8-tetramethyl-7-(2,2,2 -trichloroethoxycarbonyl)-10-undecenoate

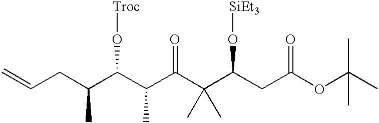

A mixture of Tert-butyl (3S,6R,7S,8S)-3-hydroxy-5-oxo-4,4,6,8-tetramethyl-7-(2,2,2 -trichloroethoxycarbonyl)-10-undecenoate (1.0 g), triethylchlorosilane (0.38 g), and imidazole (0.27 g) in 5 mL of dimethylformamide is stirred overnight, then poured into water and extracted with ether. The extract is evaporated and chromatographed on silica gel to yield the product.

### EXAMPLE 13

### (3R,6R,7S,8S)-15-hydroxy-3-(triethylsilyloxy -5-oxo-4,4,68,12,16-hexamethyl-7-( (2,2,2-trichloroethoxy)carbonyloxy)-17-(2-methyl-4-thiazolyl)heptadeca-12,16-dienoic acid

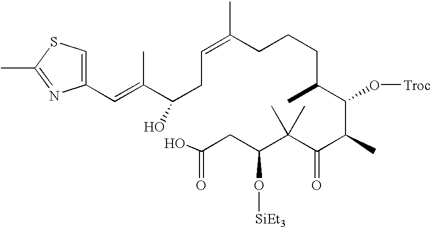

A solution of ^{t}butyl (3R,6R,7S,8S)-3-(triethylsilyloxy)-5-oxo-4,4,6,8-tetramethyl-7-((2,2,2-trichloroethoxy)carbonyloxy)-10-undecenoate thioester (10 mmol) in 80 mL of tetrahydrofuran is treated with a 0.5 M solution of 9-borabicyclo[3.3.1]nonane in tetrahydrofuran (30 mL). In a separate flask, 4-(6-iodo-3-acetoxy-2-methyl-2,6, -heptadienyl)-2-methylthiazole (13 mmol) is dissolved in 100 mL of dimethylformamide, and cesium carbonate (21 mmol) is added with vigorous stirring followed by sequential addition of triphenylarsine (11 mmol), [1,1′-bis(diphenylphosphino)ferrocene]-dichloropalladium(II) (1 mmol), and water (7 mL). After 4 hours, the borane mixture is added to the iodide mixture. After the color fades (ca. 2 hours), the mixture is poured into water, the pH is adjusted to 4.0 using 1 N HCl, and the mix is extracted with ether. The extract is washed sequentially with water and brine, dried over MgSO₄, filtered, and evaporated. The crude material is dissolved in 1:1 methanol/water and stirred with potassium carbonate to remove the acetate and thioesters. Upon completion, the mixture is concentrated to remove methanol, and the aqueous phase is adjusted to pH 4 with 1 N HCl prior to extraction with ether. The extract is dried over MgSO₄, filtered, and evaporated. The product is isolated by silica gel chromatography.

### EXAMPLE 14

### 3-O-triethylsilyl-7-O-(2,2,2-trichloroethoxycarbonyl)epothilone D

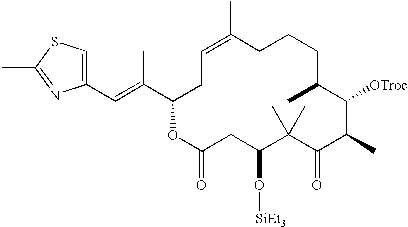

A solution of (3R,6R,7S,8S)-15-hydroxy-3-(triethylsilyloxy)-5-oxo-4,4,6,8,12,16 -hexamethyl-7-((2,2,2-trichloroethoxy)carbonyloxy)-17-(2-methyl-4-thiazolyl)heptadeca-12,16-dienoic acid (10 mmol) in tetrahydrofuran (150 mL) is treated with triethylamine (60 mmol) and 2,4,6-trichlorobenzoylchloride (50 mmol) for 15 minutes at ambient temperature, then diluted with 800 mL of toluene. This solution is added dropwise to a stirred solution of 4-dimethylaminopyridine (105 mmol) in 12 L of toluene. After addition, the mix is stirred for an additional 1 hour, then concentrated under vacuum. The product is purified by silica gel chromatography.

### EXAMPLE 15

### 3-O-(triethylsilyl)epothilone D

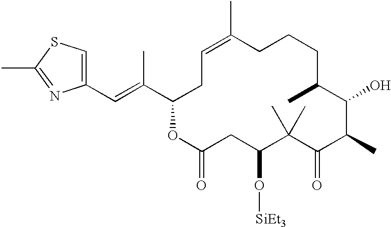

Samarium iodide is prepared by stirring a solution of samarium (3.43 mmol) and iodine (3.09 mmol) in 40 mL of tetrahydrofuran at reflux for 2.5 hours. Upon cooling to ambient temperature, 10 mg of NiI₂ is added and the mix is cooled to −78° C. A solution of 3-O-triethylsilyl-7-O-(2,2,2-trichloroethoxycarbonyl)epothilone D (0.386 mmol) in 10 mL of tetrahydrofuran is added, and the mix is stirred for 1 hour at −78° C. The reaction is quenched by addition of sat. NaHCO₃, warmed to ambient temperature, and extracted with ether. The extract is dried over MgSO₄, filtered, and evaporated. The product is purified by silica gel chromatography.

### EXAMPLE 16

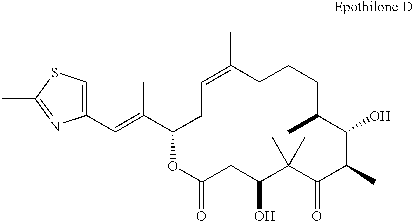

A solution of 3-O-(triethylsilyl)epothilone D (1 mmol) in 20 mL of tetrahydrofuran in a telfon reaction vessel is cooled on ice and treated with 10 mL of HF•pyridine for 90 min. The reaction is poured into sat. NaHCO₃ and extracted with CH₂Cl₂. The extract is dried over MgSO₄, filtered, and evaporated. The product is purified by silica gel chromatography.

### EXAMPLE 17

### (4R 5S,6S,13S)-5-hydroxy-3-oxo-2,2,4,6,10,14-hexamethyl-13-(^{t}butyldimethylsilyloxy)-15-(2-methyl-4-thiazolyl)pentadeca-10,14-dienoic acid δ-lactone

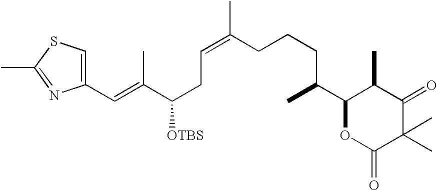

A solution of (4R,5S,6S)-5-hydroxy-3-oxo-2,2,4,6-tetramethyl-8-nonenoate δ-lactone (10 mmol) in 80 mL of tetrahydrofuran is treated with a 0.5 M solution of 9-borabicyclo[3.3.1]nonane in tetrahydrofuran (30 mL). In a separate flask, 4-(6-iodo-3-(^{t}butyldimethylsilyloxy)-2-methyl-2,6,-heptadienyl)-2-methylthiazole (13 mmol) is dissolved in 100 mL of dimethylformamide, and cesium carbonate (21 mmol) is added with vigorous stirring followed by sequential addition of triphenylarsine (11 mmol), [1,1′-bis(diphenylphosphino)ferrocene]-dichloropalladium(II) (1 mmol), and water (7 mL). After 4 hours, the borane mixture is added to the iodide mixture. After the color fades (ca. 2 hours), the mixture is poured into water and extracted with ether. The extract is washed sequentially with water and brine, dried over MgSO₄, filtered, and evaporated. The product is isolated by silica gel chromatography.

### EXAMPLE 18

### N-methoxy-N-methyl (4R,5S,6S, 13S-5-hydroxy-3-oxo-2,2,4,6,10,14-hexamethyl-13-(^{t}butyldimethylsilyloxy)-15-(2-methyl-4-thiazolyl)pentadeca-10,14-dienamide

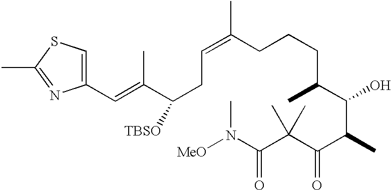

A solution of 2 M trimethylaluminum in toluene (10 mmol) is added dropwise to a suspension of N,O-dimethylhydroxylamine hydrochloride (10 mmol) in 8 mL of CH₂Cl₂ at 0° C. The resulting homogeneous solution is stirred for 30 min at ambient temperature. A solution of (4R,5S,6S,13S)-5-hydroxy-3-oxo-2,2,4,6,10,14-hexamethyl-13-(^{t}butyldimethylsilyloxy)-15-(2-methyl-4-thiazolyl)pentadeca-10,14-dienoic acid □-lactone (2 mmol) in 4 mL of CH₂Cl₂ is added over 10 min, and the resulting solution is heated at reflux until complete consumption of starting material. Upon cooling, the mixture is poured into 1 M HCl, and extracted with CH₂Cl₂. The extract is washed sequentially with 5% NaHCO₃ and brine, dried over MgSO₄, filtered, and evaporated. The product is purified by silica gel chromatography.

### EXAMPLE 19

### N-methoxy-N-methyl (4R,5S,6S,13S)-5-hydroxy-3-oxo-2,2,4,6,10,14-hexamethyl-13,15-di (^{t}butyldimethylsilyloxy)-15-(2-methyl-4-thiazolyl pentadeca-10,14-dienamide

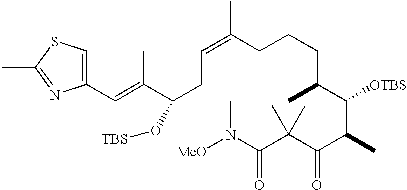

A solution of N-methoxy-N-methyl (4R,5S,6S,13S)-5-hydroxy-3-oxo-2,2,4,6,10,14-hexamethyl-13-(^{t}butyldimethylsilyloxy)-15-(2-methyl-4-thiazolyl)pentadeca-10,14-dienamide (10 mmol) in 50 mL of CH₂C1₂ is treated with 2,6-lutidine (30 mmol) and ^{t}butyldimethylsilyl triflate (25 mmol) for 1 hour. The mixture is poured into water and extracted with CH₂Cl₂. The extract is washed sequentially with 1 M phosphate buffer, pH 6, sat. NaHCO₃, and brine, then dried over MgSO₄, filtered, and evaporated. The product is isolated by silica gel chromatography.

### EXAMPLE 20

### (4R,5S,6S,13)-5-hydroxy-3-oxo-2,2,4,6, 10,14-hexamethyl-13,15-di(^{t}butyldimethylsilyl)-15-(2-methyl-4-thiazolyl)pentadeca-10,14-dienal

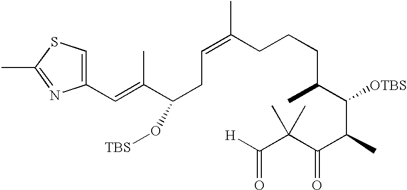

A solution of N-methoxy-N-methyl (4R,5S,6S,13S)-5-hydroxy-3-oxo-2,2,4,6,10,14 -hexamethyl-13,15-di(^{t}butyldimethylsilyloxy)-15-(2-methyl-4-thiazolyl)pentadeca-10,14-dienamide (10 mmol) in 20 mL of toluene is cooled to -78° C., and a 1.5 M solution of diisobutylaluminum hydride in toluene (50 mmol) is added dropwise over a 45 minute period. Stirring is continued until complete conversion of starting material as determined by thin-layer chromatographic analysis. Ethyl acetate (10 mL) is added, and the mixture is allowed to warm to ambient temperature. A 100-mL portion of 1 N HCl is added, and the mixture is stirred an additional 1 hour before extracting with CH₂Cl₂. The extract is dried over MgSO₄, filtered, and evaporated. The product is purified by silica gel chromatography.

### EXAMPLE 21

### ^{t}Butyl (3R,6R,7S,8S)-3-hydroxy-5-oxo-4,4,6,8,12,16-hexamethyl-7,15-di (^{t}butyldimethylsilyloxy)-17-(2-methyl-4-thiazolyl)heptadeca-12,16-dienoic acid thioester

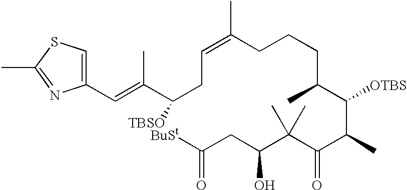

A solution of ^{t}butyl thioacetate (10 mmol) in CH₂Cl₂ (10 mL) is cooled on ice and treated sequentially with (2S,5S)-2,5-dimethylborolane triflate (11 mmol) and diisopropylethylamine (12 mmol). The mixture is cooled to −78° C., and a solution of(4R,5S,6S,13S)-5-hydroxy-3-oxo-2,2,4,6,10,14-hexamethyl-13,15-di(^{t}butyldimethylsilyloxy)-15-(2-methyl-4-thiazolyl)pentadeca-10,14-dienal (9 mmol) in 10 mL of CH₂Cl₂ is added dropwise. The mixture is stirred for 30 min, then warmed to 0° C. and kept for 1 hour. A mixture of 25 mL of 1 M phosphate buffer, pH 7, and 75 mL of methanol is added, followed by 75 mL of 2:1 methanol/50% H₂0₂, and the mixture is stirred for 1 hour at 0° C. The reaction is concentrated to a slurry in vacuo, diluted with water, and extracted with ethyl acetate. The extract is washed sequentially with 5% NaHCO₃ and brine, then dried over MgSO₄, filtered, and evaporated. The product is purified by silica gel chromatography.

### EXAMPLE 22

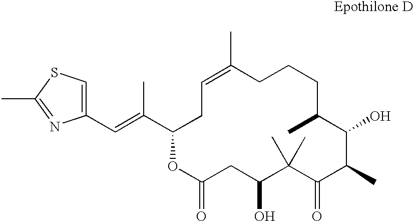

A solution of ^{t}butyl (3R,6R,7S,8S)-3-hydroxy-5-oxo-4,4,6,8, 12,16-hexamethyl-7,15-di (^{t}butyldimethylsilyloxy)-17-(2-methyl-4-thiazolyl)heptadeca-12,16-dienoic acid thioester (1 mmol) in 20 mL of tetrahydrofuran is cooled on ice and treated with anhydrous tetrabutylammonium fluoride (5 mmol). Upon completion, the reaction is poured into water and extracted with CH₂Cl₂. The extract is washed sequentially with water, sat. NaHCO₃, and brine. The extract is dried over MgSO₄, filtered, and evaporated. The product is purified by silica gel chromatography.

### EXAMPLE 23

### Preparation of (2R,3S,4S, 5R)-3,5-dihydroxy-2,4-dimethyl-6-octenoic acid δ-lactone

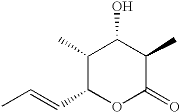

(2R,3S, 4S,5R)-3,5-dihydroxy-2,4-dimethyl-6-octenoic acid δ-lactone is made by providing 2-methyl-3-hydroxyl-4-hexenoiate N acetylcysteamine thioester to a functional PKS system comprising DEBS1 and a releasing domain wherein the ketosynthase domain of module 1 has been inactivated, fermented according to the procedure of Example 4.

### EXAMPLE 24

### Preparation of (2R 3R 4S 5R)-3,5-dihydroxy-2,4-dimethyl-6-octenoic acid δ-lactone

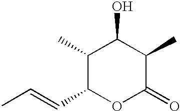

(2R,3S,4S,5R)-3,5-dihydroxy-2,4-dimethyl-6-octenoic acid δ-lactone is made by providing 2-methyl-3-hydroxyl-4-hexenoiate N acetylcysteamine thioester to a functional PKS system comprising DEBS1 and a releasing domain wherein the ketosynthase domain of module 1 has been inactivated and where the ketoreductase (“KR”) domain of module 2 of DEBS has been replaced with the ketoreductase domain of module 2 of rapamycin, fermented according to the procedure of Example 4.

### EXAMPLE 25

### Preparation of (2R,3S,4S,5S-3,5-dihydroxy-2,4-dimethyl-6-oxo-6-hexanoic acid δ-lactone

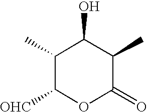

A solution of (2R,3S,4S,5R)-3,5-dihydroxy-2,4-dimethyl-6-octenoic acid δ-lactone (1.84 g) in 10 mL of CH₂Cl₂ is cooled to −78° C. and a stream of ozone is bubbled through until a blue color persists. The mixture is swept with a stream of nitrogen gas until the blue color dissipates, then treated with dimethylsulfide (2 mL) and warmed to ambient temperature and concentrated. The product is isolated by silica gel chromatography.

### EXAMPLE 26

### Preparation of (2R,3S,4S)-3,5-dihydroxy-2,4-dimethyl-6-pentanoic acid δ-lactone

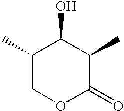

A solution of (2R,3S,4S,5S)-3,5-dihydroxy-2,4-dimethyl-6-oxo-6-hexanoic acid δ-lactone (1.72 g) and tris(triphenylphosphine)rhodium chloride (9.25 g) in 100 mL of benzene is heated at reflux for 8 hours, then evaporated. The product is isolated by silica gel chromatography.

### EXAMPLE 27

### Preparation of (2R,3R,4S,5R)-3,5-dihydroxy-2 4,6-trimethyl-6-heptenoic acid δ-lactone

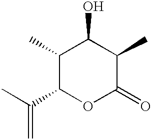

(2R,3R,4S,5R)-3,5-dihydroxy-2,4,6-trimethyl-6-heptenoic acid δ-lactone is made by providing 2,4-dimethyl-3-hydroxy-4-pentenoate N-acetylcysteamine thioester to a functional PKS system comprising DEBSl and a releasing domain wherein the ketosynthase domain of module 1 has been inactivated and where the ketoreductase (“KR”) domain of module 2 of DEBS has been replaced with the ketoreductase domain of module 2 of rapamycin, fermented according to the procedure of Example 4.

### EXAMPLE 28

### N-methoxy-N-methyl (2R,3S,4S)-3 5-dihydroxy-2,4-dimethylpentanamide

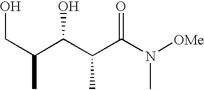

A solution of 2 M trimethylaluminum in toluene (10 mmol) is added dropwise to a suspension of N,O-dimethylhydroxylamine hydrochloride (10 mmol) in 8 mL of CH₂Cl₂ at 0° C. The resulting homogeneous solution is stirred for 30 min at ambient temperature. A solution of (2R,3S,4S)-3,5-dihydroxy-2,4-dimethyl-6-pentanoic acid δ-lactone (2 mmol) in 4 mL of CH₂Cl₂ is added over 10 min, and the resulting solution is heated at reflux until complete consumption of starting material. Upon cooling, the mixture is poured into 1 M HCl, and extracted with CH₂Cl₂. The extract is washed sequentially with 5% NaHCO₃ and brine, dried over MgSO₄, filtered, and evaporated. The product is purified by silica gel chromatography.

### EXAMPLE 29

### N-methoxy-N-methyl (2S,3S,4)-3-hydroxy-2,4-dimethyl-5-((4-methoxybenzyl) oxy)pentanamide (“Common precursor”)

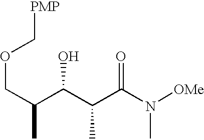

A solution of N-methoxy-N-methyl (2R,3S,4S)-3,5-dihydroxy-2,4-dimethylpentanamide (1 mmol) in 5 mL of methanol is treated with dibutyltin oxide (250 mg) at reflux for 1 hour, then treated with 4-methoxybenzyl chloride (250 mg). The mixture is evaporated, and the product is isolated by chromatography on silica gel.

### EXAMPLE 30

### (2S,3S,4S,5R)-2,4,6-trimethylhept-6-en-1,3,5-triol

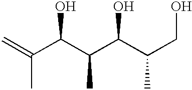

A solution of (2R,3R,4S,5R)-3,5-dihydroxy-2,4,6-trimethyl-6-heptenoic acid δ-lactone (1 mmol) in 1.5 mL of tetrahydrofuran is added dropwise to a suspension of lithium aluminum hydride (2 mmol) in 3 mL of tetrahydrofuran cooled on ice. After stirring for 1 hour, the mixture is warmed to ambient temperature and stirred for 24 hours. The reaction is then cooled on ice and treated sequentially with water and 15% KOH, then stirred vigorously for 24 hours at ambient temperature. The solids are removed by filtration, and the eluent is concentrated under vacuum. The product is purified by silica gel chromatography.

### EXAMPLE 31

### (2S,3S,4S,5R)-2,4,6-triimethylhept-6-en-1,3,5-triol 1,3-(4-methoxybenzylidene)acetal

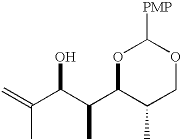

A mixture of (2S,3S,4S,5R)-2,4,6-trimethylhept-6-en-1,3,5-triol (1 mmol) and 4-methoxybenzaldehyde dimethylacetal (1.2 mmol) in 5 mL of CH₂Cl₂ is treated with camphorsulfonic acid (0.05 mmol) for 12 hours. Saturated NaHCO₃ is added, and the mixture is extracted with CH₂Cl₂. The extract is washed with brine, dried over NaSO₄, filtered, and evaporated. The product is purified by silica gel chromatography.

### EXAMPLE 32

### (2S 3S,4S,5R)-5-(^{t}butyldimethylsilyloxy)-2,4,6-triimethylhept-6-en-1,3-diol 1,3-(4-methoxybenzylidene)acetal

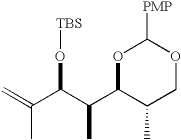

A mixture of (2S,3S,4S,5R)-2,4,6-triimethylhept-6-en-1,3,5-triol 1,3-(4-methoxybenzylidene)acetal (10 mmol), tert-butyldimethylsilyl chloride (12 mmol), and imidazole (20 mmol) in 25 mL of dimethylformamide is stirred for 24 hours at ambient temperature, then poured into water and extracted with ether. The extract is dried over MgSO₄, filtered, and evaporated. The product is purified by silica gel chromatography

### EXAMPLE 33

### Alternate preparation of (2S,3S,4R,5S)-2,4,6-trimethyl-5,7-dihydroxy-3-( ^{t}butyldimethylsilyloxy)-1-heptanol 5,7-(4-methoxybenzylidene)acetal

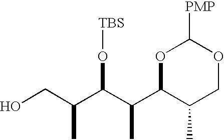

A solution of (2S,3S,4S,5R)-5-(tert-butyldimethylsilyloxy)-2,4,6-triimethylhept-6-en-1,3-diol 1,3-(4-methoxybenzylidene)acetal, (10 mmol) and tris(triphenylphosphine)rhodium chloride (0.5 mmol) in 10 mL of tetrahydrofuran is treated with a 1 M solution of catecholborane in tetrahydrofuran (30 mmol) for 8 hours at ambient temperature. Ethanol (3 mL) is added followed by 25 mL of sat. NaHCO₃ and 10 mL of 30% H₂O₂. The mix is vigorously stirred for 2 hours, then diluted with sat. Na₂SO₃ and extracted with ether. The extract is washed with brine, dried over Na₂SO₄, filtered, and evaporated. The product is purified by silica gel chromatography.

### EXAMPLE 34

### (2R,3S,4R,5S)-2,4,6-trimethyl-5,7-dihydroxy-3-(^{t}butyldimethylsilyloxy)-1-iodoheptane 5,7-(4-methoxybenzylidene) acetal

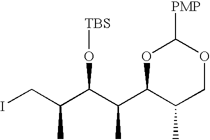

A solution of (2S,3S,4R,5S)-2,4,6-trimethyl-5,7-dihydroxy-3-(^{t}butyldimethylsilyloxy)-1-heptanol 5,7-(4-methoxybenzylidene) acetal (10 mmol) in 70 mL of 1:2 benzene/ether is treated with triphenylphosphine (15 mmol), imidazole (15 mmol), and iodine (15 mmol) with vigorous stirring. After 1 hour, the mix is diluted with ether and washed sequentially with sat. sodium thiosulfate and brine, dried over Na₂SO₄, filtered, and evaporated. The product is purified by silica gel chromatography.

### EXAMPLE 35

### (2R,3R,4S,5R)-5-hydroxy-2,4-dimethyl-3-(tert-butyldimethylsilyloxy -6-octenoic acid δ-lactone

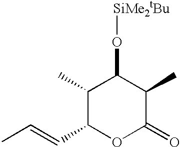

A solution of (2R,3R,4S,5R)-3,5-dihydroxy-2,4-dimethyl-6-octenoic acid δ-lactone (10 mmol), 2,6-lutidine (15 mmol), and tert-butyldimethylsilyl triflate (11 mmol) in 10 mL of CH₂Cl₂ is stirred for 1 hour. The mixture is washed with water, then dried over MgSO₄, filtered, and evaporated. The product is purified by silica gel chromatography.

### EXAMPLE 36

### (2R,3S,4S,5R,7S)-5-hydroxy-8-oxo-2,4-dimethyl-3,7-di(tert-butyldimethylsilyloxy)-8octanoic acid δ-lactone

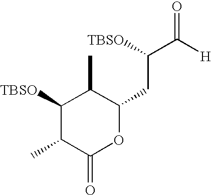

A solution of (2R,3S,4S,5R,7S)-5-hydroxy-2,4,9-trimethyl-3,7-di(tert-butyldimethylsilyloxy 8-decenoic acid δ-lactone (10 mmol) in 150 mL of CH₂Cl₂ is cooled to −78° C., and a stream of ozone in oxygen is bubbled through until a blue color persists. The mixture is purged with a stream of air for 10 minutes, then triphenylphosphine (11 mmol) is slowly added. The mixture is allowed to warm to ambient temperature and stirred for 1 hour, then concentrated. The product is isolated by silica gel chromatography.

### EXAMPLE 37

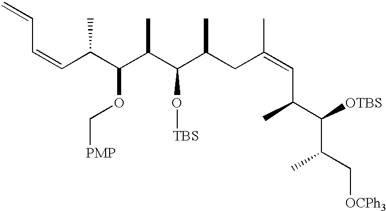

A solution of the product of Example 51 (10 mmol) in 100 mL of CH₂Cl₂ is treated with Dess-Martin periodinane (12 mmol) and NaHCO₃ (30 mmol) for 3 hours, then quenched by addition of sat. NaS₂O₃ and sat. NaHCO₃ solutions. The mixture is extracted with ether, and the extract is washed with water and brine, dried over MgSO₄, filtered, and evaporated. The crude aldehyde is used immediately in the next step.

A 1.7 M solution of tert-butyllithium in pentane (20 mmol) is added to a −78° C. solution of allyldiphenylphosphine (20 mmol) in 60 mL of degassed tetrahydrofuran. The mixture is stirred for 5 min, then warmed to 0° C., stirred for 30 min, and recooled to −78° C. Titanium tetraisopropoxide (20 mmol) is added. After 30 min, a cold solution of the aldehyde from above in 35 mL of tetrahydrofuran is added and stirred for 1 hour. The solution is warmed to 0° C., and methyl iodide (100 mL) is added. The solution is stirred at ambient temperature for 16 hours, quenched by addition of phosphate buffer, pH 7, and extracted with CH₂Cl₂. The extract is washed with brine, dried over MgSO₄, filtered, and evaporated. The product is purified by silica gel chromatography.

### EXAMPLE 38

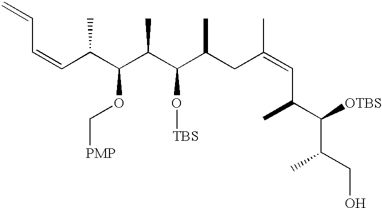

Methanol (0.75 mL) is added to a 0° C. solution of chlorocatecholborane (72.5 mmol) in CH₂Cl₂ (20 mL). The resulting solution is added dropwise to a solution of the product of Example 52 (10 mmol) in CH₂Cl₂ at 0° C., and the reaction is monitored by thin-layer chromatography. Once the reaction is ca. 90% complete, the mixture is treated with sat. NaHCO₃ and stirred for 15 minutes. The mixture is extracted with ether, and the extract is washed with brine, dried with MgSO₄, filtered, and evaporated. The product is purified by silica gel chromatography.

### EXAMPLE 39

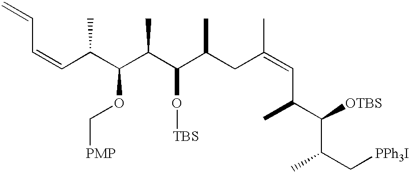

A solution of iodine (20 mmol) in 50 mL of ether is added dropwise to a solution of the alcohol product of Example 53 (10 mmol), triphenylphosphine (25 mmol), and imidazole (25 mmol) in 200 mL of 1:1 ether/benzene cooled to 0° C. The resulting suspension is stirred for 30 min, then poured into 750 mL of 1:1 water/hexanes. The phases are separated, and the aqueous phase is extracted with hexanes. The organic phases are combined and ashed with sat. aq. Na₂S₂₃, water, and brine, then dried over MgSO₄, filtered, and evaporated to yield a slurry. The slurry is loaded onto a short column of silica gel with a small volume of CH₂Cl₂, and the product is rapidly eluted using a mixture of 2% ether+0.05% Et₃N in hexanes. The eluent is concentrated in vacuo to yield the crude iodide. This is dissolved in 25 mL of 7:3 benzene/toluene, treated with 1 mL of diisopropylethylamine and 12.5 g of triphenylphosphine, and loaded into a high pressure apparatus and subjected to a pressure of 12.8 kbar for 14 days. The mixture is then concentrated and chromatographed on silica gel to provide the product, which is dried by repeated evaporated from benzene followed by heated under vacuum at 50° C. for 12 hours.

### EXAMPLE 40

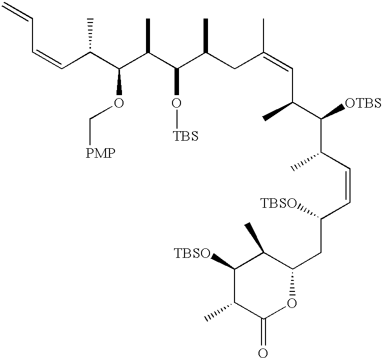

The phosphonium salt product of Example 54 (11 mmol) is dissolved in 60 mL of tetrahydrofuran, and is placed under argon atmosphere, and is cooled to −20° C. A 1.0 M solution of sodium bis(trimethylsilyl)amide in tetrahydrofuran (10.5 mmol) is added, the mixture is stirred for 15 min, warmed to 0° C., stirred for 30 min, then recooled to −25° C. A solution of the aldehyde from Example 47 (11 mmol) in 30 mL of tetrahydrofuran is added over 15 minutes, then the mixture is warmed slowly to −10° C. and quenched with a mixture of sat. NH₄Cl and ether. The mixture is extracted with ether, and the extract is dried over MgSO₄, filtered, and evaporated. The product is isolated by silica gel chromatography.

### EXAMPLE 41

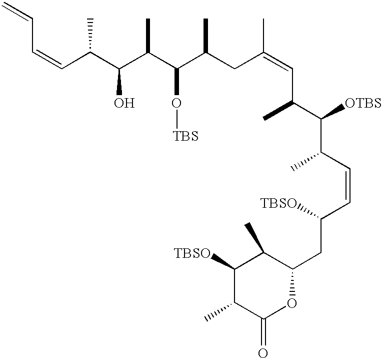

A solution of the product of Example 55 (10 mmol) in 100 mL of CH₂Cl₂ is cooled to 0° C. and treated with water (5 mL) and 2,3-dichloro-5,6-dicyano-1,4-benzoquinone (12 mmol) for 10 minutes. The mixture is warmed to ambient temperature, stirred for 5 minutes, treated with saturated NaHCO₃, and extracted with CH₂Cl₂. The extract is washed with water and brine, then dried over MgSO₄, filtered, and evaporated. The product is isolated by silica gel chromatography.

### EXAMPLE 42

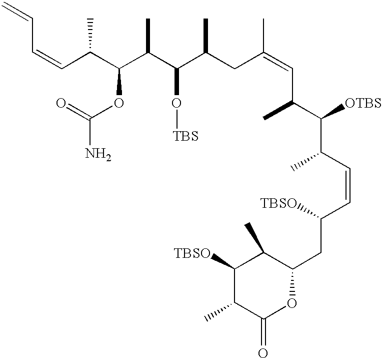

A solution of the alcohol from Example 56 (10 mmol) in CH₂Cl₂ (100 mL) is treated with trichloroacetylisocyanate (12 mmol) for 1 hour at ambient temperature. The solution is loaded onto neutral alumina, and eluted from the alumina after 4 hours using ethyl acetate. The eluent is concentrated, and the product is purified by silica gel chromatography.

### EXAMPLE 43

### (+)-Discodermolide

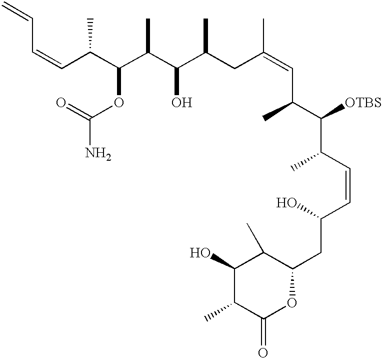

The product of Example 57 (1 mmol) is dissolved in 300 mL of methanol and stirred for 15 minutes, then 3 N HCl (200 mL) is added in 20-mL portions at such a rate so as to minimize precipitation. After completion of this addition, additional 3 N HCl (100 mL) is added in 4 portions at 15 minute intervals. After 8 hours, a final portion of 3 N HCl (100 mL) is added, the solution is stirred for 2 hours, and finally diluted with ethyl acetate (2000 mL). The phases are separated, and the aquoeus phase is extracted with ethyl acetate. The organic extracts are combined, washed with sat. NaHCO₃ and brine, dried with Na₂SO₄, filtered, and evaporated. The product is chromatographed on silica gel, then crystallized from acetonitrile.

### EXAMPLE 44

### (7Z)-(2R,3S,4R,5S,6S)-5-hydroxy-3-(^{t}butyldimethylsilyloxy)-2,4,6-trimethyldeca-7,9-dienoate δ-lactone

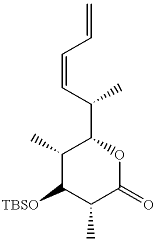

A solution of allyldiphenylphosphine (10.8 mmol) in 35 mL of tetrahydrofuran is cooled to −78° C. and treated with a 1.7 M solution of tert-butyllithium in pentane (10.8 mmol) for 5 minutes. The solution is warmed to 0° C., stirred for an additional 30 minutes, then recooled to −78° C. Titanium tetraisopropoxide (10.8 mmol) is added and stirring is continued for 30 minutes prior to addition of a −78° C. solution of (2R,3S,4R,5S,6R)-5-hydroxy-3 -(^{t}butyldimethylsilyloxy)-7-oxo-2,4,6-trimethylheptano ate δ-lactone (5.4 mmol) in 20 mL of tetrahydrofuran. After 1 hour, the mixture is warmed to 0° C., iodomethane (3.4 mL) is added, and the mixture is allowed to stir at ambient temperature for 16 hours.Phosphate buffer, pH 7.0, is added and the mixture is extracted with CH₂Cl₂. The extract is washed with brine, dried over MgSO₄, filtered, and evaporated. The product is purified by silica gel chromatography.

### EXAMPLE 45

### (7Z)-(2S,3R,4R,5S,6S)-5-((4-methoxybenzyl)oxy)-3-((^{t}butyldimethylsilyl) oxy)-2,4,6-trimethyldeca-7,9-diene-1-ol

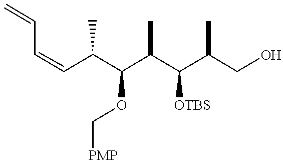

A solution of 4-methoxybenzyl alcohol (11 mmol) in 10 mL of tetrahydrofuran is added dropwise to a suspension of sodium hydride (12 mmol) in 50 mL of tetrahydrofuran. After cessation of gas evolution, the mixture is treated with a solution of (7Z)-(2R,3S,4R,5S,6S)-5 -hydroxy-3-(^{t}butyldimethylsilyloxy)-2,4,6-trimethyldeca-7,9-dienoate δ-lactone (10 mmol) in 10 mL of tetrahydrofuran. The reaction is monitored by thin-layer chromatography. Upon disappearance of the lactone, the mixture is treated with 4-methoxybenzyl bromide (12 mmol) and tetrabutylammonium iodide (1 mmol) for 5 hours at ambient temperature. The mixture is quenched by addition of sat. aq. NH₄Cl and extracted with ether. The extract is washed with brine, dried over MgSO₄, filtered, and evaporated to yield the crude protected ester. The crude ester is dissolved in 10 mL of tetrahydrofuran and added to a 1.0 M solution of lithium aluminum hydride (10 mL) at 0° C. After stirring for 3 hours, the mixture is quenched with sat. aq. NH4Cl and extracted with ether. The extract is washed with brine, dried over MgSO₄, filtered, and evaporated to yield the crude alcohol. The product is purified by silica gel chromatography.

### EXAMPLE 46

### (7Z)-(2S,3R,4R,5S,6S)-5-((4-methoxybenzyl)oxy-3-((^{t}butyldimethylsilyl)oxy )-2,4,6-trimethyldeca-7,9-dienenitrile

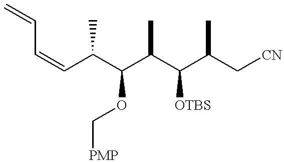

A solution of (7Z)-(2S,3R,4R,5S,6S)-5-((4-methoxybenzyl)oxy)-3-( (^{t}butyldimethylsilyl)oxy)-2,4,6-trimethyldeca-7,9-diene-1-ol (10 mmol) in 70 mL of 1:2 benzene/ether is treated with triphenylphosphine (15 mmol), imidazole (15 mmol), and iodine (15 mmol) with vigorous stirring. After 1 hour, the mix is diluted with ether and washed sequentially with sat. sodium thiosulfate and brine, dried over Na₂SO₄, filtered, and evaporated. The crude iodide is dissolved in methylsulfoxide (50 mL) and treated with sodium cyanide (15 mmol). The solution is diluted with water and extracted with ether. The extract is washed with brine, dried over MgSO₄, filtered, and evaporated. The product is isolated by silica gel chromatography.

### EXAMPLE 47

### (7Z)-(2S,3R,4R,5S,6S)-5-((4-methoxybenzyl)oxy)-3-((^{t}butyldimethylsilyl)oxy )-2,4,6-trimethyldeca-7,9-dieneal

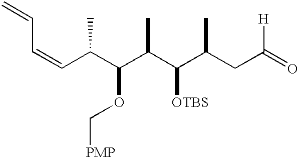

A solution of (7Z)-(2S,3k,4R,5S,6S)-5-((4-methoxybenzyl)oxy)-3-((^{t}butyldimethylsilyl)oxy)-2,4,6-trimethylundeca-7,9-dienenitrile (10 mmol) in tetrahydrofuran (10 mL) is cooled to −60° C. and treated with a 1.0 M solution of diisobutylaluminum hydride in toluene (12 mL). After 1 hour, the mix is allowed to warm to ambient temperature and kept an additional 3 hours before addition of sat. aq. NH4Cl. After 30 minutes, the mixture is carefully acidified with 5% H₂SO₄ and immediately extracted with ether. The extract is washed with brine, dried over MgSO₄, filtered, and evaporated. The product is isolated by silica gel chromatography.

### EXAMPLE 48

### 3,7,11,17-tetra-(O-tert-butyldimethylsilyl)-19-(O-descarbamoyloxy)-19-aminodiscodermolide

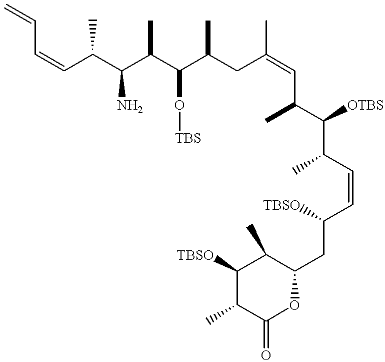

A solution of 3,7,11,17-tetra-(O-tert-butyldimethylsilyl)-19-(O-descarbamoyl)-9-epi-discodermolide (1 mmol) in 50 mL of DMF is cooled to 0° C. and treated with diisopropylethylamine (1.5 mmol) and methanesulfonic anhydride (1.5 mmol). After 1 hour, the mixture is treated with sodium azide (10 mmol). The reaction is continued for 2 hours at 50° C., then poured into sat. NaHCO3 and extracted with CH2Cl₂. The extract is washed with brine, dried over MgSO₄, filtered, and evaporated. The 19-azido-product is purified by silica gel chromatography.

The 19-azide is dissolved in 10 mL of THF and treated with 5 mL of a 1 M solution of trimethylphosphine in THF. After 2 hours, water is added and the mixture is stirred overnight, then concentrated to dryness. The 19-amine is isolated by silica gel chromatography.

### EXAMPLE 49

### 37,11,17-tetra-(O-tert-butyldimethylsilyl)-19-(O-descarbamoyloxy)-19-(carbamoylamino)discodermolide

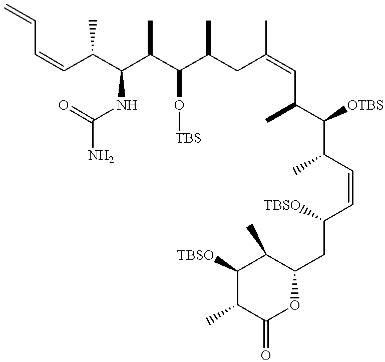

A solution of 3,7,11,17-tetra-(O-tert-butyldimethylsilyl)-19-(O-descarbamoyloxy)-19-aminodiscodermolide (1 mmol) in CH₂Cl₂ (10 mL) is treated with trichloroacetylisocyanate (1.2 mmol) for 1 hour at ambient temperature. The solution is loaded onto neutral alumina, and eluted from the alumina after 4 hours using ethyl acetate. The eluent is concentrated, and the product is purified by silica gel chromatography.

### EXAMPLE 50

### 19-(O-descarbamoyloxy)-19-(carbamoylamino)discodermolide

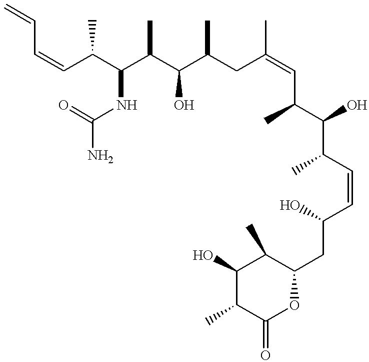

3,7,11,17-tetra-(O-tert-butyldimethylsilyl)-19-(O-descarbamoyloxy)-19-(carbamoylamino)discodermolide (1 mmol) is dissolved in 300 mL of methanol and stirred for 15 minutes, then 3 N HCl (200 mL) is added in 20-mL portions at such a rate so as to minimize precipitation. After completion of this addition, additional 3 N HCl (100 mL) is added in 4 portions at 15 minute intervals. After 8 hours, a final portion of 3 N HCl (100 mL) is added, the solution is stirred for 2 hours, and finally diluted with ethyl acetate (2000 mL). The phases are separated, and the aquoeus phase is extracted with ethyl acetate. The organic extracts are combined, washed with sat. NaHCO₃ and brine, dried with Na₂SO₄, filtered, and evaporated. The product is chromatographed on silica gel.

### EXAMPLE 51

### (±)-(2S*,3R*)-4-chloro-3-hydroxy-2-methylbutyrate N-acetylcysteamine thioester

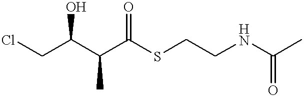

Step 1. A solution of N-propionyl-2-benzoxazolone (100.0 g) in anhydrous CH₂Cl₂ (1100 mL) is cooled to 3° C. with mechanical stirring under N₂ atmosphere. TiCl4 (58.4 mL) is added at a rate such that the internal temperature remains below 10° C. (ca. 10 minutes). The resulting yellow slurry is stirred vigorously for 40 minutes, then triethylamine (87.4 mL) is added at a rate such that the internal temperature remains below 10° C. (ca. 10 minutes). The resulting deep red solution is stirred for 80 minutes. A 1 M solution of chloroacetaldehyde in CH₂Cl₂ (1000 mL) is added at a rate such that the internal temperature remains below 10° C. (ca. 20 minutes), and the reaction is followed by thin-layer chromatography (4:1 hexanes/ethyl acetate). After stirring for 90 minutes, the reaction is quenched by addition of 450 mL of 2 N HCl. The phases are separated, and the organic phase is filtered through a pad of silica gel. The silica gel is washed with ether, and the combined organic are concentrated under vacuum to a. The product is collected by vacuum filtration and rinsed with hexanes to yield a colorless solid.

Step 2. One molar equivalent of sodium methoxide (25% w/v in methanol; ca. 150 mL) is added in a slow stream to a solution of N,S-diacetylcysteamine (173 g) in methanol (910 mL) under N₂. When half of the calculated volume has been added, the reaction is monitored by TLC (1:1 ethyl acetate/hexanes), and methoxide addition is continued until complete conversion of the N,S-diacetylcysteamine to N-acetylcysteamine. Acetic acid (50 g) is added, and the resulting solution of sodium thiolate is cannulated into a flask containing solid (±)-N-[syn-4-chloro-3-hydroxy-4-butanoyl]-2-benzoxazolone (270 g) under N₂. After 15 minutes, the reaction is quenched with solid oxalic acid dihydrate (80.4 g), filtered, and concentrated to a yellow oil. The residue is dissolved in 2:1 hexanes/ethyl acetate and submitted to batch elution chromatography on SiO₂. The silica is washed with 2:1 hexanes/ethyl acetate to remove 2-benzoxazolone, then with ethyl acetate/methanol (9:1) to elute the product thioester. Evaporation of the thioester-containing eluent yields the product.

### EXAMPLE 52

### (±)-(2S*,3R*)-3-hydroxy-2-methyl-4-(phenylthio)butyrate N-acetylcysteamine thioester

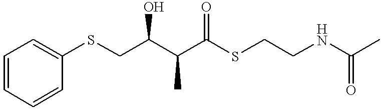

Step 1. A solution of N-propionyl-2-benzoxazolone (100.0 g) in anhydrous CH₂Cl₂ (1100 mL) is cooled to 3° C. with mechanical stirring under N₂ atmosphere. TiCl₄ (58.4 mL) is added at a rate such that the internal temperature remains below 10° C. (ca. 10 minutes). The resulting yellow slurry is stirred vigorously for 40 minutes, then triethylamine (87.4 mL) is added at a rate such that the internal temperature remains below 10° C. (ca. 10 minutes). The resulting deep red solution is stirred for 80 minutes. Phenylthioacetaldehyde (160 gm) is added at a rate such that the internal temperature remains below 10° C. (ca. 20 minutes), and the reaction is followed by thin-layer chromatography (4:1 hexanes/ethyl acetate). After stirring for 90 minutes, the reaction is quenched by addition of 450 mL of 2 N HCl. The phases are separated, and the organic phase is filtered through a pad of silica gel. The silica gel is washed with ether, and the combined organic are concentrated under vacuum to a. The product is collected by vacuum filtration and rinsed with hexanes to yield a colorless solid.

Step 2. One molar equivalent of sodium methoxide (25% w/v in methanol; ca. 150 mL) is added in a slow stream to a solution of N,S-diacetylcysteamine (173 g) in methanol (910 mL) under N₂. When half of the calculated volume has been added, the reaction is monitored by TLC (1:1 ethyl acetate/hexanes), and methoxide addition is continued until complete conversion of the N,S-diacetylcysteamine to N-acetylcysteamine. Acetic acid (50 g) is added, and the resulting solution of sodium thiolate is cannulated into a flask containing solid (±)-N-[syn-4-phenylthio-3-hydroxy-4-butanoyl]-2-benzoxazolone (340 g) under N₂. After 15 minutes, the reaction is quenched with solid oxalic acid dihydrate (80.4 g), filtered, and concentrated to a yellow oil. The residue is dissolved in 2:1 hexanes/ethyl acetate and submitted to batch elution chromatography on SiO₂. The silica is washed with 2:1 hexanes/ethyl acetate to remove 2-benzoxazolone, then with ethyl acetate/methanol (9:1) to elute the product thioester. Evaporation of the thioester-containing eluent yields the product.

### EXAMPLE 53

### Alternate Preparation of (2R,3S,4S)-3,5-dihydroxy-2,4-dimethyl-6-pentanoic acid δ-lactone

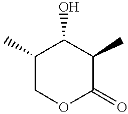

A suspension of (2R,3S,4S,5R)-3,5-dihydroxy-2,4-dimethyl-6-octenoic acid δ-lactone (1.84 g) in 10 mL of water is treated with 15 mL of 1 N sodium hydroxide and stirred until complete dissolution is obtained. The pH of the solution is adjusted to 7.0, and a 4% solution of osmium tetraoxide in water (2 mL) is added followed by sodium periodate (10 g). The mixture is stirred vigorously overnight, then treated with sodium borohydride until disappearance of aldehyde as determined by reaction of an aliquot with acidic dinitrophenylhydrazine solution. The mixture is adjusted to pH 3 and extracted with ethyl acetate. The extract is dried over MgSO₄, filtered, and evaporated to yield the product.

### EXAMPLE 54

### N-methoxy-N-methyl (2R,3S,4)-3,5-dihydroxy-2,4-dimethylpentanamide

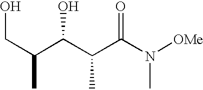

A solution of 2 M trimethylaluminum in toluene (10 mmol) is added dropwise to a suspension of N,O-dimethylhydroxylamine hydrochloride (10 mmol) in 8 mL of CH₂Cl₂ at 0° C. The resulting homogeneous solution is stirred for 30 min at ambient temperature. A solution of (2R,3S,4S)-3,5-dihydroxy-2,4-dimethyl-6-pentanoic acid δ-lactone (2 mmol) in 4 mL of CH₂Cl₂ is added over 10 min, and the resulting solution is heated at reflux until complete consumption of starting material. Upon cooling, the mixture is poured into 1 M HCl, and extracted with CH₂Cl₂. The extract is washed sequentially with 5% NaHCO₃ and brine, dried over MgSO₄, filtered, and evaporated. The product is purified by silica gel chromatography.

### EXAMPLE 55

### N-methoxy-N-methyl (2R,3S,4S)-3,5-dihydroxy-2,4-dimethylpentanamide 3,5-(4 -methoxybenzylidene)acetal

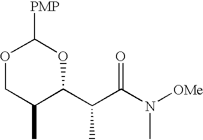

A solution of N-methoxy-N-methyl (2R,3S,4S)-3,5-dihydroxy-2,4-dimethylpentanamide (10 mmol) and 4-methoxybenzaldehyde dimethylacetal (12 mmol) in 50 mL of CH₂Cl₂ is treated with anhydrous zinc chloride (1 mmol) for 20 hours. Saturated NaHCO₃ is added, and the mixture is extracted with CH₂Cl₂. The extract is washed with brine, dried over NaSO₄, filtered, and evaporated. The product is purified by silica gel chromatography.

### EXAMPLE 56

### Alternate preparation of N-methoxy-N-methyl (2S,3S,4S)-3-hydroxy-2,4-dimethyl-5-((4-methoxybenzyl)oxy)pentanamide

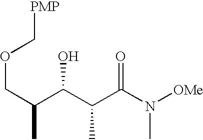

A solution of N-methoxy-N-methyl (2R,3S,4S)-3,5-dihydroxy-2,4-dimethylpentanamide 3,5 -(4-methoxybenzylidene)acetal (1 mmol) in 5 mL of tetrahydrofuran is treated with 1.0 M HCl in ether and sodium cyanoborohydride. See e.g., Garegg & Hultberg, Carbohydrate Res. 1981, 93:123.

When complete, the reaction is quenched by addition of sat. NaHCO₃ and extracted with ether. The extract is washed sequentially with 1 N HCl, sat. NaHCO₃, and brine, then dried over MgSO₄, filtered, and evaporated. The product is purified by silica gel chromatography.

### EXAMPLE 57

### (2S,3S,4S,5R)-2,4-dimethyloct-6-en-1,3,5-triol

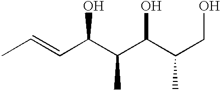

A solution of (2R,3R,4S,5R)-3,5-dihydroxy-2,4-dimethyl-6-octenoic acid δ-lactone (1 mmol) in 1.5 mL of tetrahydrofuran is added dropwise to a suspension of lithium aluminum hydride (2 mmol) in 3 mL of tetrahydrofuran cooled on ice. After stirring for 1 hour, the mixture is warmed to ambient temperature and stirred for 24 hours. The reaction is then cooled on ice and treated sequentially with water and 15% KOH, then stirred vigorously for 24 hours at ambient temperature. The solids are removed by filtration, and the eluent is concentrated under vacuum. The product is purified by silica gel chromatography.

### EXAMPLE 58

### (2S,3S,4S,5R)-3,5-dihydroxy-2,4-dimethyl-1-((4-methoxybenzyl)oxy)oct-6-ene

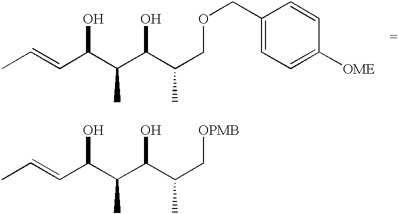

A solution of (2S, 5S,4S,5R-2,4-dimethyloct-6-en-1,3,5-triol (10 mmol) and freshly prepared 4-methoxybenzyl 2,2,2-trichloroacetimidate (13 mmol) in 15 mL of 1:2 CH₂Cl₂/cyclohexane is treated with pyridinium p-toluenesulfonate (0.5 mmol) at 0° C. After 3 h, the mix is warmed to ambient temperature and stirred for an additional 48 hours. The solvent is evaporated, and the product is purified by silica gel chromatography.

### EXAMPLE 59

### (2S,3S,4S,5R)-2,4-dimethyloct-6-en-1,3,5-triol 1,3-(4-methoxybenzylidene) acetal

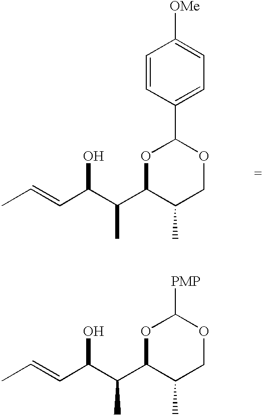

A mixture of (2S,3S,4S,5R)-2,4-dimethyloct-6-en-1,3,5-triol (1 mmol) and 4-methoxybenzaldehyde dimethylacetal (1.2 mmol) in 5 mL of CH₂Cl₂ is treated with camphorsulfonic acid (0.05 mmol) for 12 hours. Saturated NaHCO₃ is added, and the mixture is extracted with CH₂Cl₂. The extract is washed with brine, dried over NaSO₄, filtered, and evaporated. The product is purified by silica gel chromatography.

### EXAMPLE 60

### (2S,3S,4S,5R)-3,5-dihydroxy-2,4-dimethyl-1-((4-methoxybenzyl)oxy)oct-6-ene Alternate preparation

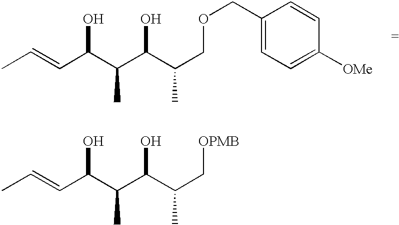

A solution of (2S,3S,4S,5R)-3,5-dihydroxy-2,4-dimethyloct-6-en-1-ol 1,3-(4-methoxyphenyl)acetal (1 mmol) in 5 mL of tetrahydrofuran is treated with 1.0 M HCl in ether and sodium cyanoborohydride. See, Garegg & Hultberg, Carbohydrate Res. 1981, 93:123.

When complete, the reaction is quenched by addition of sat. NaHCO₃ and extracted with ether. The extract is washed sequentially with 1 N HCl, sat. NaHCO₃, and brine, then dried over MgSO₄, filtered, and evaporated. The product is purified by silica gel chromatography.

### EXAMPLE 61

### (2R,3S,4S)-2,4-dimethyl-3-hydroxy-5-((4-methoxybenzyl)oxy)pentanoic acid

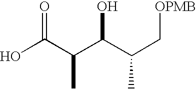

A solution of (2S,3S,4S,5R)-3,5-dihydroxy-2,4-dimethyl-1-((4-methoxybenzyl)oxy)oct-6 -ene (1 mmol) in CCl₄ and acetonitrile is treated with sodium periodate in the presence of catalytic ruthenium dichloride. See, Webster et al., J. Org. Chem. 1987, 52:689-91.

### EXAMPLE 62

### (2R,3S,4S)-2,4-dimethyl-3,5-dihydroxypentanal 3,5-(4-methoxybenzylidene) acetal

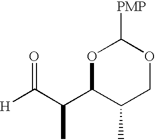

A solution of (2S,3S,4S,5R)-2,4-dimethyloct-6-en-1,3,5-triol 1,3-(4-methoxybenzylidene) acetal (2.6 mmol) in a mix of 2,2-dimethylpropanol (30 mL), acetone (65 mL), and water (16 mL) is treated with osmium tetraoxide (0.08 mmol) and N-methylmorpholine N-oxide (13 mmol) for 11 hours at ambient temperature. The solution is diluted with 1 M NaH₂PO₄ and extracted with CH₂Cl₂. The extract is dried over Na₂SO₄, filtered, and evaporated to yield a crude triol intermediate. This material is dissolved in a mix of tetrahydrofuran (120 mL) and water (25 mL), and sodium metaperiodate (13 mmol) is added. After stirring vigorously for 24 hours, the mix is diluted with sat. NaHCO₃ and extracted with CH₂Cl₂. The extract is dried over Na₂SO₄, filtered, and evaporated. The product is purified by silica gel chromatography.

### EXAMPLE 63

### (4R)-4-benzyl-3-[(2R,3S,4S,5S)-2,4,6-trimethyl-3,5,7-trihydroxyheptanoyl 5,7-methoxybenzylidene) acetal]-2-oxazolidinone

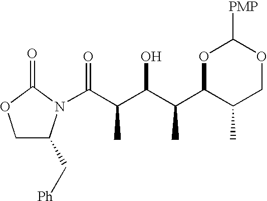

A solution of (4R)-4-benzyl-3-propionyl-2-oxazolidinone (10 mmol) in 10 mL of CH₂Cl₂ is cooled on ice and treated with 1.0 M solution of dibutylboron triflate in CH₂Cl₂ (11 mmol) followed by triethylamine (12 mmol). After stirring for 30 min, the solution is cooled to −78° C. and a solution of (2R,3S,4S)-2,4-dimethyl-3,5-dihydroxypentanal 3,5-(4-methoxybenzylidene) acetal (9 mmol) in 10 mL of CH₂Cl₂ is added dropwise. The mixture is stirred for 30 min, then warmed to 0° C. and kept for 1 hour. A mixture of 25 mL of 1 M phosphate buffer, pH 7, and 75 mL of methanol is added, followed by 75 mL of 2:1 methanol/50% H₂O₂, and the mixture is stirred for 1 hour at 0° C. The reaction is concentrated to a slurry in vacuo, diluted with water, and extracted with ethyl acetate. The extract is washed sequentially with 5% NaHCO₃ and brine, then dried over MgSO₄, filtered, and evaporated. The product is purified by silica gel chromatography.

### EXAMPLE 64

### (4R)-4-benzyl-3-[(2R,3S,4R,5S)-2,4,6-trimethyl-5,7-dihydroxy-3-( ^{t}butyldimethylsilyloxy)heptanoyl 5,7-(4-methoxybenzylidene) acetal]-2-oxazolidinone

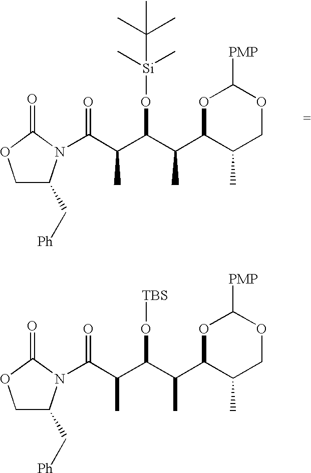

A solution of (4R)-4-benzyl-3-[(2R,3S,4S,5S)-2,4,6-trimethyl-3,5,7 -trihydroxyheptanal 5,7-(4-methoxybenzylidene) acetal]-2-oxazolidinone (10 mmol) in 10 mL of CH₂Cl₂ is treated with ^{t}butyldimethylsilyl triflate (12 mmol) and 2,6-lutidine (20 mmol). After 1 hour, the mixture is poured into sat. NaHCO₃ and extracted with CH₂Cl₂. The extract is washed with brine, dried over Na₂SO₄, filtered, and evaporated. The product is purified by silica gel chromatography.

### EXAMPLE 65

### Alternate Preparation of (2S,3S,4R,5S)-2,4,6-trimethyl-5,7-dihydroxy-3-( ^{t}butyldimethylsilyloxy)-l-heptanol 5,7-(4-methoxybenzylidene) acetal

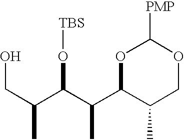

A solution of (4R)-4-benzyl-3-[(2R,3S,4R,5S)-2,4,6-trimethyl-5,7-dihydroxy-3 -(^{t}butyldimethylsilyloxy)heptanoyl 5,7-(4-methoxybenzylidene) acetal]-2-oxazolidinone (10 mmol) in tetrahydrofuran (100 mL) is cooled to −30° C., and ethanol (20 mmol) was added followed by addition of a 2 M solution of lithium borohydride in tetrahydrofuran (20 mmol) over 15 min. After stirring for 1 hour on ice, the mix is warmed to ambient temperature and stirred an additional 12 hours. The mix is diluted with ether and 40 mL of 1 N NaOH is added. After 2 hours, the phases are separated and the organic phase is washed with brine, dried with Na₂SO₄, filtered, and evaporated. The product is purified by silica gel chromatography.

### EXAMPLE 66

### (2S,3S,4R,5S)-2,4,6-trimethyl-5,7-dihydroxy-3-(^{t}butyldimethylsilyloxy)heptanal 5,7-(4-methoxybenzylidene) acetal

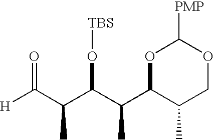

Oxalyl chloride (26 mmol) is added over 1 hour to a solution of methylsulfoxide (56 mmol) in CH₂Cl₂ (100 mL) cooled to −78° C. After an additional 15 minutes, a −78° C. solution of (2S,3S,4R,5S)-2,4,6-trimethyl-5,7-dihydroxy-3-(^{t}butyldimethylsilyloxy)-1-heptanol 5,7-(4-methoxybenzylidene) acetal (18 mmol) in CH₂Cl₂ (5 mL) is added over 15 min. After stirring for 30 min, diisopropylethylamine (86 mmol) is added over 15 min. The reaction is stirred an additional 30 min at −78° C., then allowed to warm to ambient temperature over 1 hour. After addition of 1 N NaHSO₄, the mix is diluted with ether, washed with water, dried with MgSO₄, filtered, and evaporated. The product is purified by silica gel chromatography.

### EXAMPLE 67

### (3S,4R,5R,6S)-3,5,7-trimethyl-6,8-dihydroxy-4-(^{t}butyldimethylsilyloxy)oct-1-ene 6,8-(4-methoxybenzylidene) acetal

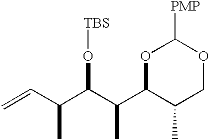

A 1.6 M solution of butyllithium in hexane (12 mmol) is added dropwise to a suspension of methyltriphenylphosphonium bromide (12 mmol) in tetrahydrofuran. After 1 hour, a solution of (2S,3S,4R,5S)-2,4,6-trimethyl-5,7-dihydroxy-3-(^{t}butyldimethylsilyloxy)-heptanal 5,7-(4-methoxybenzylidene) acetal (10 mmol) is added and stirred for 12 hours. The mixture is cooled to ambient temperature and concentrated. The residue is dissolved in 1:1 ether/hexane, filtered, and concentrated. The product is purified by silica gel chromatography.

### EXAMPLE 68

### (4S,5R,6R,7S)-4,6,8-trimethyl-7,9-dihydroxy-5-(butyldimethylsilyloxy)non-2-ene 7,9-( 4-methoxybenzylidene) acetal

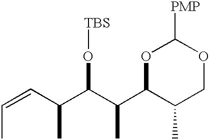

A 1.6 M solution of butyllithium in hexane (12 mmol) is added dropwise to a suspension of ethyltriphenylphosphonium bromide (12 mmol) in tetrahydrofuran. After 1 hour, a solution of (2S,3S, 4R, 5S)-2,4,6-trimethyl-5,7-dihydroxy-3-(^{t}butyldimethylsilyloxy)-heptanal 5,7-(4-methoxybenzylidene) acetal (10 mmol) is added and stirred for 12 hours. The mixture is cooled to ambient temperature and concentrated. The residue is dissolved in 1:1 ether/hexane, filtered, and concentrated. The product is purified by silica gel chromatography.

### EXAMPLE 69

### (3S,4R,5R,6S)-3,5,7-trimethyl-6,8-dihdroxy-4-(^{t}butyldimethylsilyloxy -1-octa 6,8-(4-methoxybenzylidene)acetal

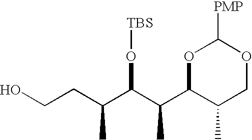

Borane-dimethylsulfide (23 mmol) is added to a solution of 2-methyl-2-butene (45 mmol) in tetrahydrofuran (60 mL) at 0° C., and the solution is stirred for 2 hours. This solution of disiamylborane is added to a solution of (3S,4R,5R,6S)-3,5,7-trimethyl-6,8-dihydroxy-4-( ^{t}butyldimethylsilyloxy)oct-1-ene 6,8-(4-methoxybenzylidene) acetal (7.5 mmol) in 90 mL of tetrahydrofuran at 0° C. After stirring for 90 minutes, ethanol (3 mL) is added followed by 25 mL of sat. NaHCO₃ and 10 mL of 30% H₂O₂. The mix is warmed to ambient temperature with vigorous stirring over 2 hours, then diluted with sat. Na₂SO₃ and extracted with ether. The extract is washed with brine, dried over Na₂SO₄, filtered, and evaporated. The product is purified by silica gel chromatography.

### EXAMPLE 70

### (4S,5R,6R,7S)-4,6,8-trimethyl-7,9-dihydroxy-5-(butyldimethylsilyloxy)nonan-2-ol 7,9-(4-methoxybenzylidene) acetal

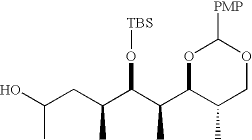

Borane-dimethylsulfide (23 mmol) is added to a solution of 2-methyl-2-butene (45 mmol) in tetrahydrofuran (60 mL) at 0° C., and the solution is stirred for 2 hours. This solution of disiamylborane is added to a solution of (4S,5R,6R,7S)-4,6,8-trimethyl-7,9-dihydroxy-5-( ^{t}butyldimethylsilyloxy)non-2-ene 7,9-(4-methoxybenzylidene) acetal (7.5 mmol) in 90 mL of tetrahydrofuran at 0° C. After stirring for 90 minutes, ethanol (3 mL) is added followed by 25 mL of sat. NaHCO₃ and 10 mL of 30% H₂O₂. The mix is warmed to ambient temperature with vigorous stirring over 2 hours, then diluted with sat. Na₂SO₃ and extracted with ether. The extract is washed with brine, dried over Na₂SO₄, filtered, and evaporated. The product is purified by silica gel chromatography.

### EXAMPLE 71

### (3S,4R,5R,6S)-1-triphenylphosphonium-3,5,7-trimethyl-6,8-dihydroxy-4-( ^{t}butyldimethylsilyloxy)octane 6,8-(4-methoxybenzylidene)acetal iodide

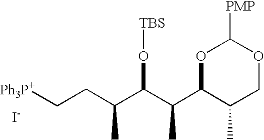

A solution of (3S,4R,5R,6S)-3,5,7-trimethyl-6,8-dihydroxy-4-(^{t}butyldimethylsilyloxy)-1 -octanol 6,8-(4-methoxybenzylidene)acetal (10 mmol) in 70 mL of 1:2 benzene/ether is treated with triphenylphosphine (15 mmol), imidazole (15 mmol), and iodine (15 mmol) with vigorous stirring. After 1 hour, the mix is diluted with ether and washed sequentially with sat. sodium thiosulfate and brine, dried over Na₂SO₄, filtered, and evaporated. The crude iodide is dissolved in tetrahydrofuran (50 mL) and treated with triphenylphosphine (15 mmol) at reflux. The solution is cooled to ambient temperature, and hexane is added to crystallize the phosphonium salt.

### EXAMPLE 72

### (4S,5R,6R,7S)-2-triphenylphosphonium-4,6,8-trimethyl-7,9-dihydroxy-5-( ^{t}butyldimethylsilyloxy)nonan-2-ol 7,9-(4-methoxybenzylidene)acetal iodide

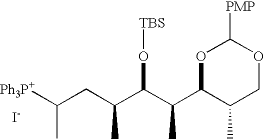

A solution of (4S,5R,6R,7S)-4,6,8-trimethyl-7,9-dihydroxy-5-(^{t}butyldimethylsilyloxy )-nonan-2-ol 7,9-(4-methoxybenzylidene) acetal (10 mmol) in 70 mL of 1:2 benzene/ether is treated with triphenylphosphine (15 mmol), imidazole (15 mmol), and iodine (15 mmol) with vigorous stirring. After 1 hour, the mix is diluted with ether and washed sequentially with sat. sodium thiosulfate and brine, dried over Na₂SO₄, filtered, and evaporated. The crude iodide is dissolved in tetrahydrofuran (50 mL) and treated with triphenylphosphine (15 mmol) at reflux. The solution is cooled to ambient temperature, and hexane is added to crystallize the phosphonium salt.

### EXAMPLE 73

### (2R,3S,4S,5R)-2,4-dimethyl-3-(tert-butyldimethylsilyloxy)oct-6-en-1 ,5-diol

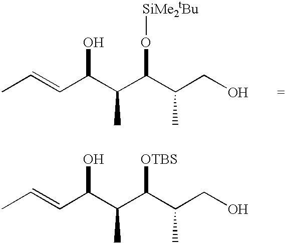

A solution of (2R,3R,4S,5R)-5-hydroxy-2,4-dimethyl-3-(tert-butyldimethylsilyloxy)-6-octenoic acid δ-lactone (1 mmol) in 1.5 mL of tetrahydrofuran is added dropwise to a suspension of lithium aluminum hydride (2 mmol) in 3 mL of tetrahydrofuran cooled on ice. After stirring for 1 hour, the mixture is warmed to ambient temperature and stirred for 24 hours. The reaction is then cooled on ice and treated sequentially with water and 15% KOH, then stirred vigorously for 24 hours at ambient temperature. The solids are removed by filtration, and the eluent is concentrated under vacuum. The product is purified by silica gel chromatography.

### EXAMPLE 74

### (2R,3R,4S,5R)-1-((4-methoxyphenyl)methoxy)-2,4-dimethyl-3-( tert-butyldimethylsilyloxy oct-6-en-5-ol

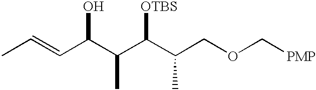

A solution of (2R,3S,4S,5R)-2,4-dimethyl-3-(tert-butyldimethylsilyloxy)oct-6-en-1,5-diol (10 mmol) and freshly prepared 4-methoxybenzyl 2,2,2-trichloroacetimidate (13 mmol) in 15 mL of 1:2 CH₂Cl₂/cyclohexane is treated with pyridinium p-toluenesulfonate (0.5 mmol) at 0° C. After 3 h, the mix is warmed to ambient temperature and stirred for an additional 48 hours. The solvent is evaporated, and the product is purified by silica gel chromatography.

### EXAMPLE 75

### (2R,3S,4S)-5-((4-methoxyphenyl)methoxy)-2,4-dimethyl-3-(tert-butyldimethylsilyloxy)pentanal

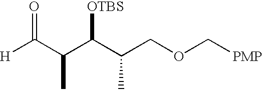

A solution of (2R,3R,4S,5R)-1-((4-methoxyphenyl)methoxy)-2,4-dimethyl-3-( tert-butyldimethylsilyloxy)oct-6-en-5-ol (2.6 mmol) in a mix of 2,2-dimethylpropanol (30 mL), acetone (65 mL), and water (16 mL) is treated with osmium tetraoxide (0.08 mmol) and N-methylmorpholine N-oxide (13 mmol) for 11 hours at ambient temperature. The solution is diluted with 1 M NaH₂PO₄ and extracted with CH₂Cl₂. The extract is dried over Na₂SO₄, filtered, and evaporated to yield a crude triol intermediate. This material is dissolved in a mix of tetrahydrofuran (120 mL) and water (25 mL), and sodium metaperiodate (13 mmol) is added. After stirring vigorously for 24 hours, the mix is diluted with sat. NaHCO₃ and extracted with CH₂Cl₂. The extract is dried over Na₂SO₄, filtered, and evaporated. The product is purified by silica gel chromatography.

### EXAMPLE 76

### (3S,4S,5S)-1-iodo-6-((4-methoxyphenyl)methoxy)-3,5-dimethyl-4-( tert-butyldimethylsilyloxy)-1-hexene

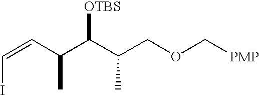

A 1.6 M solution of butyllithium in hexanes (37 mmol) is added to a suspension of methyltriphenylphosphonium iodide (36 mmol) in tetrahydrofuran (200 mL) over 10 minutes. After an additional 10 minutes, the solution is added over 15 minutes to a −78° C. solution of iodine (32 mmol) in 300 mL of tetrahydrofuran. The resulting yellow slurry is stirred for 5 min, then warmed to −23° C. and kept for 10 minutes. A 1 M solution of sodium hexamethyldisilazide in tetrahydrofuran (31 mmol) is added over 8 minutes, and the mix is stirred an additional 15 minutes prior to addition of a solution of (2R,3S,4S)-5-( (4-methoxyphenyl)methoxy)-2,4-dimethyl-3-(tert-butyldimethylsilyloxy)-pentanal (18 mmol) in 50 mL of tetrahydrofuran. After 10 min, the mix is warmed to ambient temperature and stirred for 3 hours. Methanol (10 mL) is added, and the mixture is concentrated under vacuum and the residue filtered through a pad of silica gel using 1:1 ethyl acetate/hexanes. The filtrate is washed sequentially with sat. Na₂S₂O₃ and brine, dried over MgSO₄, filtered, and concentrated. The product is purified by silica gel chromatography.

### EXAMPLE 77

### (4S,5S,6S)-2-iodo-7-((4-methoxyphenyl)methoxy)-4,6-dimethyl-5-( tert-butyldimethylsilyloxy)-2-heptene

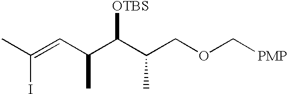

A 1.6 M solution of butyllithium in hexanes (37 mmol) is added to a suspension of ethyltriphenylphosphonium iodide (36 mmol) in tetrahydrofuran (200 mL) over 10 minutes. After an additional 10 minutes, the solution is added over 15 minutes to a −78° C. solution of iodine (32 mmol) in 300 mL of tetrahydrofuran. The resulting yellow slurry is stirred for 5 min, then warmed to −23° C. and kept for 10 minutes. A 1 M solution of sodium hexamethyldisilazide in tetrahydrofuran (31 mmol) is added over 8 minutes, and the mix is stirred an additional 15 minutes prior to addition of a solution of (2R,3S,4S)-5-(( 4-methoxyphenyl)methoxy)-2,4-dimethyl-3-(tert-butyldimethylsilyloxy)-pentanal (18 mmol) in 50 mL of tetrahydrofuran. After 10 min, the mix is warmed to ambient temperature and stirred for 3 hours. Methanol (10 mL) is added, and the mixture is concentrated under vacuum and the residue filtered through a pad of silica gel using 1:1 ethyl acetate/hexanes. The filtrate is washed sequentially with sat. Na₂S₂O ₃ and brine, dried over MgSO₄, filtered, and concentrated. The product is purified by silica gel chromatography.

### EXAMPLE 78

### (2R,3R,4S,5R)-1-(triphenylmethoxy)-2,4-dimethyl-3-(tert-butyldimethylsilyloxy) oct-6-en-5-ol

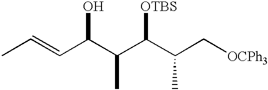

A solution of (2R,3S,4S,5R)-2,4-dimethyl-3-(tert-butyldimethylsilyloxy)oct-6-en-1,5-diol (10 mmol) and triphenylmethyl chloride (11 mmol) in pyridine (25 mL) is stirred for 12 hours. The solvent is evaporated, and the product is purified by silica gel chromatography.

### EXAMPLE 79

### (2R,3S,4S)-5-(phenylmethoxy)-2,4-dimethyl-3-(tert-butyldimethylsilyloxy)pentanal

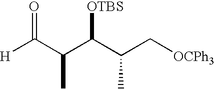

A solution of (2R,3R,4S,5R)-1-(triphenylmethoxy)-2,4-dimethyl-3-( tert-butyldimethylsilyloxy)oct-6-en-5-ol (2.6 mmol) in a mix of 2,2-dimethylpropanol (30 mL), acetone (65 mL), and water (16 mL) is treated with osmium tetraoxide (0.08 mmol) and N-methylmorpholine N-oxide (13 mmol) for 11 hours at ambient temperature. The solution is diluted with 1 M NaH₂PO₄ and extracted with CH₂C1₂. The extract is dried over Na₂SO₄, filtered, and evaporated to yield a crude triol intermediate. This material is dissolved in a mix of tetrahydrofuran (120 mL) and water (25 mL), and sodium metaperiodate (13 mmol) is added. After stirring vigorously for 24 hours, the mix is diluted with sat. NaHCO₃ and extracted with CH₂Cl₂. The extract is dried over Na₂SO₄, filtered, and evaporated. The product is purified by silica gel chromatography.

### EXAMPLE 80

### (2R,3S,4S,5R)-5-hydroxy-2,4-dimethyl-3-(tert-butyldimethylsilyloxy)-6-octenoic acid δ-lactone

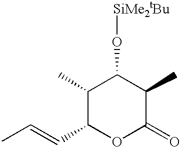

A solution of (2R,3S,4S,5R)-3,5-dihydroxy-2,4-dimethyl-6-octenoic acid δ-lactone (10 mmol), 2,6-lutidine (15 mmol), and tert-butyldimethylsilyl triflate (11 mmol) in 10 mL of CH₂Cl₂ is stirred for 1 hour. The mixture is washed with water, then dried over MgSO₄, filtered, and evaporated. The product is purified by silica gel chromatography.

### EXAMPLE 81

### N-methoxy-N-methyl (2R,3S,4S,5R)-5-hydroxy-2,4-dimethyl-3-(tert-butyldimethylsilyloxy)-6-octenamide

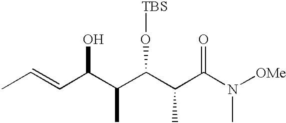

A solution of 2 M trimethylaluminum in toluene (10 mmol) is added dropwise to a suspension of N,O-dimethylhydroxylamine hydrochloride (10 mmol) in 8 mL of CH₂Cl₂ at 0° C. The resulting homogeneous solution is stirred for 30 min at ambient temperature. A solution of (2R,3S,4S,5R)-5-hydroxy-2,4-dimethyl-3-(tert-butyldimethylsilyloxy)-6-octenoic acid δ-lactone (2 mmol) in 4 mL of CH₂Cl₂ is added over 10 min, and the resulting solution is heated at reflux until complete consumption of starting material. Upon cooling, the mixture is poured into 1 M HCl, and extracted with CH₂Cl₂. The extract is washed sequentially with 5% NaHCO₃ and brine, dried over MgSO₄, filtered, and evaporated. The product is purified by silica gel chromatography.

### EXAMPLE 82

### N-methoxy-N-methyl (2R,3S,4R)-2,4-dimethyl-3-(tert-butyldimethylsilyloxy)pentanamide

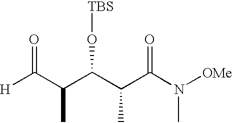

A solution of N-methoxy-N-methyl (2R,3S,4S,5R)-5-hydroxy-2,4-dimethyl-3-( tert-butyldimethylsilyloxy)-6-octenamide (2.6 mmol) in a mix of 2,2-dimethylpropanol (30 mL), acetone (65 mL), and water (16 mL) is treated with osmium tetraoxide (0.08 mmol) and N-methylmorpholine N-oxide (13 mmol) for 11 hours at ambient temperature. The solution is diluted with 1 M NaH₂PO₄ and extracted with CH₂Cl₂. The extract is dried over Na₂SO₄, filtered, and evaporated to yield a crude triol intermediate. This material is dissolved in a mix of tetrahydrofuran (120 mL) and water (25 mL), and sodium metaperiodate (13 mmol) is added. After stirring vigorously for 24 hours, the mix is diluted with sat. NaHCO₃ and extracted with CH₂Cl₂. The extract is dried over Na₂SO₄, filtered, and evaporated. The product is purified by silica gel chromatography.

### EXAMPLE 83

### (2R,3S,4S,5R)-5-hydroxy-7-oxo-2,4,9-trimethyl-3-(tert-butyldimethylsilyloxy)-8 -decenoic acid δ-lactone

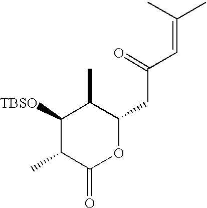

A 1.0 M solution of TiCl₄ in CH₂Cl₂ (10 mmol) is added dropwise to a −78° C. solution of N-methoxy-N-methyl (2R,3S,4R)-2,4-dimethyl-3-(tert-butyldimethylsilyloxy)-pentanamide (10 mmol) in 80 mL of CH₂Cl₂. To this mix is added 4-methyl-2-(trimethylsilyloxy)-1,3-pentadiene (20 mmol) and the mixture is stirred for 2 hours at −78° C. The reaction is quenched by addition of 2:1 phosphate buffer (pH 8) and sat. NaHCO₃ (250 mL) and warmed to ambient temperature. The mix is extracted with CH₂Cl₂ and the extract is dried over MgSO₄, filtered, and evaporated. The residue is dissolved in 50 mL of 1:1 CH₂Cl₂/hexane and treated with trichloroacetic acid (15 mmol) on ice for 5 hours. The mix is diluted with hexane, washed sequentially with water, phosphate buffer (pH 8), and brine, then dried with MgSO₄, filtered, and evaporated. The product is isolated by silica gel chromatography.

### EXAMPLE 84

### (2R,3S,4S,5R,7S)-5,7-dihydroxy-2,4,9-trimethyl-3-(tert-butyldimethylsilyloxy)-8 -decenoic acid δ-lactone

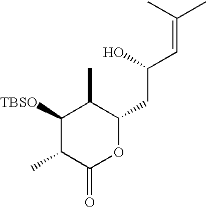

A solution of (2R,3S,4S,5R)-5-hydroxy-7-oxo-2,4,9-trimethyl-3-(tert-butyldimethylsilyl-oxy )-8-decenoic acid δ-lactone (10 mmol) in toluene (250 mL) is cooled to −95° C. and treated with a 1.0 M solution of K-selectride (potassium tri-sec-butylborohydride) in tetrahydrofuran (12 mmol). After 2 hours, 0.5 mL of acetic acid is added, the solution is warmed to ambient temperature, and a mixture of phosphate buffer (pH 7) (325 mL) and 30% H₂O₂ (15 mL) is added. After stirring for 2 hours, the mix is extracted with CH₂Cl₂ and the extract is dried with MgSO₄, filtered, and concentrated. The product is isolated by silica gel chromatography.

### EXAMPLE 85

### (2R,3S,4S,5R,7S)-5-hydroxy-2,4,9-trimethyl-3 7-di(tert-butyldimethylsilyloxy)-8-decenoic acid δ-lactone

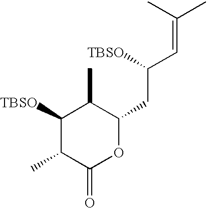

A solution of (2R,3S,4S,5R,7S)-5,7-dihydroxy-2,4,9-trimethyl-3-(tert-butyldimethylsilyloxy )-8-decenoic acid δ-lactone (10 mmol), tert-butyldimethylsilyl chloride (20 mmol), and imidazole (30 mmol) in 50 mL of dimethylformamide is stirred at ambient temperature for 12 hours, then diluted with ether, washed with water, dried over MgSO₄, filtered, and concentrated. The product is isolated by silica gel chromatography.

### EXAMPLE 86

### Alternate preparation of (2R,3S,4S,5R,7S)-5,7-dihydroxy-2,4,9-trimethyl-3-( tert-butyldimethylsilyloxy)-8-decenoic acid δ-lactone

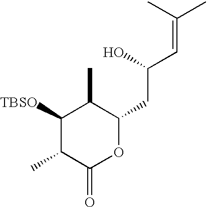

A 1.0 M solution of TiCl₄ in CH₂Cl₂ (10 mmol) is added dropwise to a −78° C. solution of N-methoxy-N-methyl (2R,3S,4R)-2,4-dimethyl-3-(tert-butyldimethylsilyloxy)-pentanamide (10 mmol) in 80 mL of CH₂Cl₂. To this mix is added 4-methyl-2-(trimethylsilyloxy)-1,3-pentadiene (20 mmol) and the mixture is stirred for 2 hours at −78° C. The reaction is quenched by addition of 2:1 phosphate buffer (pH 8) and sat. NaHCO₃ (250 mL) and warmed to ambient temperature. The mix is extracted with CH₂Cl₂ and the extract is dried over MgSO₄, filtered, and evaporated. The residue is dissolved in 50 mL of acetonitrile and added to a −40° C. solution of tetramethylammonium triacetoxyborohydride (80 mmol) and acetic acid (44 mL) in 44 mL of acetonitrile which had been allowed to stir for 30 minutes at ambient temperature prior to cooling. The reaction is allowed to proceed for 18 hours at 40° C., then is quenched by addition of 0.5 M aqueous sodium potassium tartrate and warmed to ambient temperature. The mix is extracted with CH₂Cl₂ and the extract is dried over MgSO₄, filtered, and evaporated. The residue is dissolved in 50 mL of 1:1 CH₂Cl₂/hexane and treated with trichloroacetic acid (15 mmol) on ice for 5 hours. The mix is diluted with hexane, washed sequentially with water, phosphate buffer (pH 8), and brine, then dried with MgSO₄, filtered, and evaporated. The product is isolated by silica gel chromatography.

### EXAMPLE 87

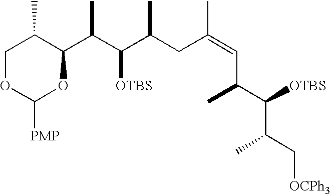

A 1 M solution of sodium hexamethyldisilazide in tetrahydrofuran (10 mmol) is added to a suspension of (4S,5R,6R,7S)-2-triphenylphosphonium-4,6,8-trimethyl-7,9-dihydroxy-5-( ^{t}butyldimethylsilyloxy)nonan-2-ol 7,9-(4-methoxybenzylidene)acetal iodide (10 mmol) in 10 mL of tetrahydrofuran. After 15 minutes, a solution of (2R,3S,4S)-5-(triphenylmethoxy)-2,4-dimethyl-3-(tert-butyldimethylsilyloxy)pentanal (10 mmol) is added and the mix is stirred for 3 hours. The mixture is concentrated under vacuum and the residue filtered through a pad of silica gel using 1:1 ethyl acetate/hexanes. The filtrate is concentrated. The product is purified by silica gel chromatography.

### EXAMPLE 88

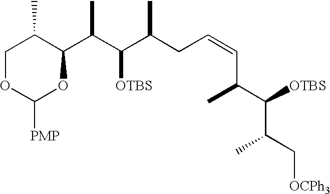

A 1 M solution of sodium hexamethyldisilazide in tetrahydrofuran (10 mmol) is added to a suspension of (3S,4R,5R,6S)-1-triphenylphosphonium-3,5,7-trimethyl-6,8-dihydroxy-4-( ^{t}butyldimethylsilyloxy)octane 6,8-(4-methoxybenzylidene)acetal iodide (10 mmol) in 10 mL of tetrahydrofuran. After 15 minutes, a solution of (2R,3S,4S)-5-(triphenylmethoxy)-2,4-dimethyl-3-(tert-butyldimethylsilyloxy)pentanal (10 mmol) is added and the mix is stirred for 3 hours. The mixture is concentrated under vacuum and the residue filtered through a pad of silica gel using 1:1 ethyl acetate/hexanes. The filtrate is concentrated. The product is purified by silica gel chromatography.

### EXAMPLE 89

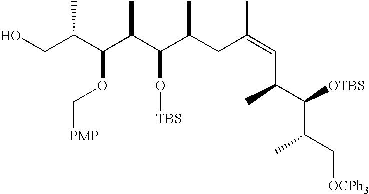

A 1 M solution of diisobutylaluminum hydride in toluene (30 mmol) is added to a 0° C. solution of the product of Example 49 (10 mmol) in 100 mL of CH₂Cl₂, and the mixture is stirred for 5 hours. Aqueous phosphate buffer (pH 7.0) is added dropwise to quench, the mixture is diluted with 100 mL of CH₂Cl₂, poured into 100 mL of saturated sodium potassium tartrate, and extracted with CH₂Cl₂. The extract is dried over MgSO₄, filtered, and evaporated. The crude product is purified by silica gel chromatography.

### EXAMPLE 90

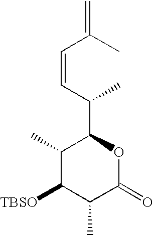

A solution of 4-methoxybenzyl alcohol (11 mmol) in 10 mL of tetrahydrofuran is added dropwise to a suspension of sodium hydride (12 mmol) in 50 mL of tetrahydrofuran. After cessation of gas evolution, the mixture is treated with a solution of (7Z)-(2R,3S,4R,5S,6S)-5-hydroxy-3-(^{t}butyldimethylsilyloxy)-2,4,6-trimethyldeca-7,9-dieno ate δ-lactone (10 mmol) in 10 mL of tetrahydrofuran. The reaction is monitored by thin-layer chromatography. Upon disappearance of the lactone, the mixture is cooled to 0° C. and treated with trifluoromethanesulfonic anhydride (12 mmol). After formation of the triflate, the mixture is treated with 2,3-dichloro-1,5-dicyano-1,4-benzoquinone (12 mmol) and water for 10 minutes at 0° C., then warmed to ambient temperature prior to addition of triethylamine (12 mmol). The reaction is monitored by thin-layer chromatography. When complete, the mixture is quenched by addition of sat. aq. NaHCO₃ and extracted with ether. The extract is washed with brine, dried over MgSO₄, filtered, and evaporated. The product is purified by silica gel chromatography.

The compounds of the present invention generally include a plurality of chiral centers and optionally a double bond. Although preferred embodiments (preferred isomers) are used to illustrate the invention, the present invention encompasses all stereo and geometric isomers. All scientific and patent publications referenced herein are hereby incorporated by reference. The invention having now been described by way of written description and example, those of skill in the art will recognize that the invention can be practiced in a variety of embodiments, that the foregoing description and example is for purposes of illustration and not limitation of the following claims.

What is claimed is:

1. A method for making a naturally-occurring polyketide comprising
fermenting a host cell containing an expression vector,
said vector comprising a recombinant gene encoding a polyketide synthase,
said synthase comprising modules from at least two different naturally-occurring polyketide synthases, wherein at least one of the naturally-occurring polyketide synthases does not naturally produce said polyketide, and
optionally isolating said polyketide from the fermentation medium.

2. The method of claim 1, wherein the polyketide is an epothilone analog or a discodermolide analog.

3. A method for making a first compound useful in synthesizing a second compound, wherein
said second compound contains four or more chiral centers, and
said first compound contains two or more chiral centers,
said method comprising expressing in a recombinant host cell a recombinant, non-naturally occurring polyketide synthase that produces said first compound.

4. The method of claim 3, wherein the first compound contains at least 3 chiral centers, and the second compound contains at least 5 chiral centers.

5. The method of claim 3, wherein the second compound contains at least 10 chiral centers.

6. The method of claim 3, wherein the recombinant, non-naturally occurring PKS is either a portion of a naturally occurring PKS gene or is composed of portions of two or more naturally occurring PKS genes.

7. The method of claim 3, wherein the second compound is an epothilone analog or a discodermolide analog.

8. The method of claim 3, wherein said first compound is selected from the group consisting of:

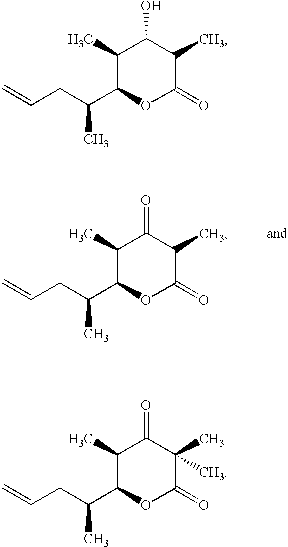

9. The method of claim 3, wherein said first compound is selected from the group consisting of 14-chloro-14-desmethyl-6-deoxyerythronolide B and 14-desmethyl-6-deoxy-14-(phenylthio)erythronolide B.

10. The method of claim 3, wherein said first compound is selected from the group consisting of: 14-chloro-14-desmethyl-6-deoxy-8-hydroxyerythronolide B and 14-desmethyl-6-deoxy-8-hydroxy-14-(phenylthio)erythronolide B.

11. A compound of the formula: 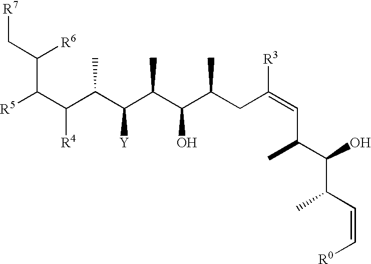
wherein
R⁰ is C1-C8 alkyl, C1-C8 alkenyl, C1-C8 alkynyl, aryl, 2-phenylethyl, 2-(3-hydroxyphenyl)ethyl, or a group of the formula 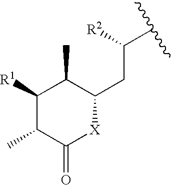
wherein R¹ and R² are each independently hydrogen, hydroxyl, or a hydroxyl protecting group; and X is O, NH, or N-alkyl;
R³ is hydrogen, C₁-C₁₀ alkyl or aryl;
R⁴, R⁵, R⁶, and R⁷ are each hydrogen, or R⁴ and R⁵ together form a double bond and R⁶ and R⁷ together form a double bond; and
Y is hydroxyl, amino, —OC(═O)NH₂ or —NHC(═O)NH₂, with the proviso that when R³ is hydrogen or C₁-C₆ alkyl that: (i) at least one of R¹ and R²is not hydroxyl, or (ii) R⁴, R⁵, R⁶, and R⁷ are each hydrogen, or (iii) X is nitrogen, or (iv) Y is hydroxyl, amino, or —NHC(═O)NH₂, or (v) any combination of (i) through (iv).

12. The compound of claim 11 that is: 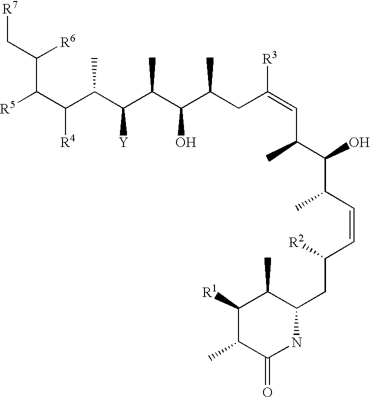
wherein
R¹ and R² are each independently hydrogen, hydroxyl, or a hydroxyl protecting group;
R³ is hydrogen, C₁C₁₀ alkyl or aryl;
R⁴, R⁵, R⁶, and R⁷ are each hydrogen, or R⁴ and R⁵ together form a double bond and R⁶ and R⁷ together form a double bond; and,
Y is hydroxyl, amino, —OC(═O)NH₂ or —NHC(═O)NH₂.

13. The compound of claim 11 that is: 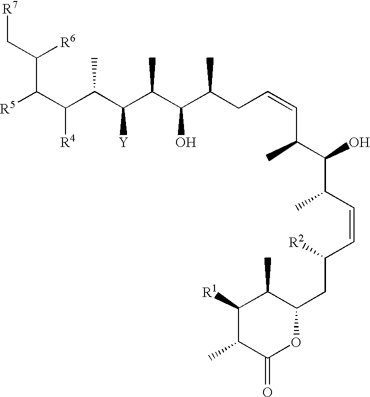
wherein
R¹ and R² are each independently hydrogen, hydroxyl, or a hydroxyl protecting group;
R⁴, R⁵, R⁶, and R⁷ are each hydrogen, or R⁴ and R⁵ together form a double bond and R⁶ and R⁷ together form a double bond; and,
Y is hydroxyl, amino, —OC(═O)NH₂ or —NHC(═O)NH₂, provided at least one of R¹ and R² is not hydroxyl.

14. The compound of claim 11 that is: 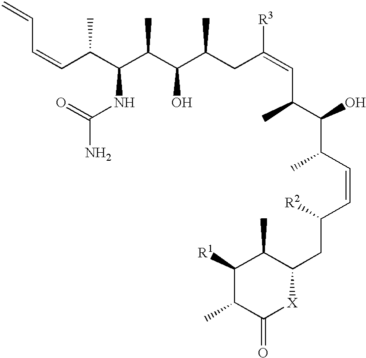
wherein
R¹ and R² are each independently hydrogen, hydroxyl, or a hydroxyl protecting group;
R³ is hydrogen, C₁-C₁₀ alkyl or aryl; and,
X is oxygen or nitrogen.

15. The compounds of claim 11 that are selected from the group consisting of:

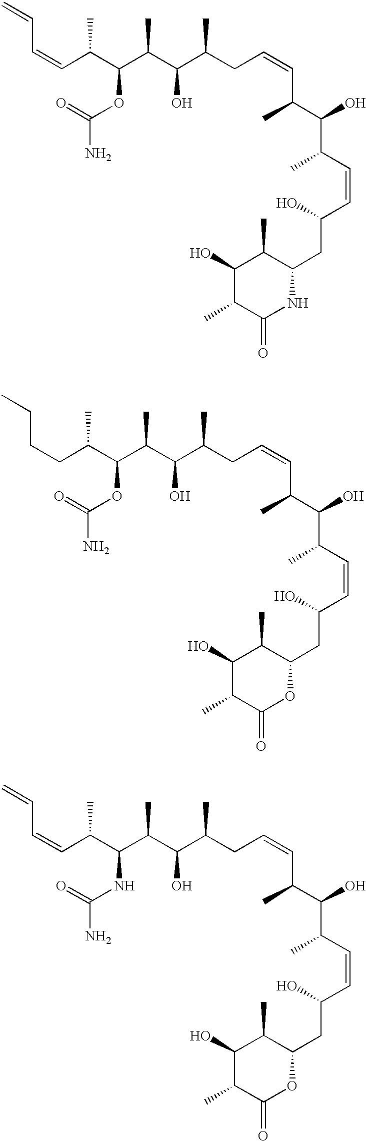

16. The compounds of claim 11 that are selected from the group consisting of: [chemical structure]
